(19) United States
(12) Patent Application Publication
(10) Pub. No.: US 2026/0097187 A1
(43) Pub. Date: Apr. 9, 2026

(54) SYSTEM AND METHODS FOR A GUIDEWIRE WITH A MULTI-EDGE PROFILE

(71) Applicant: Piraeus Medical, Inc., Rochester, MN (US)

(72) Inventor: Jared Hutar, Cromwell, MN (US)

(21) Appl. No.: 19/354,674

(22) Filed: Oct. 9, 2025

Related U.S. Application Data
(60) Provisional application No. 63/705,288, filed on Oct. 9, 2024.

(51) Int. Cl. A61M 25/09; A61M 25/00

(52) U.S. Cl. CPC A61M 25/09; A61M 25/005; A61M 2025/09075; A61M 2025/09133; A61M 2205/0222; A61M 2205/0238

(57) ABSTRACT

A guidewire is disclosed that includes a multi-edge profile configured to improve torque transmission and reduce frictional engagement during intravascular navigation. In some embodiments, the multi-edge profile includes a square cross-section twisted into a helical structure. In some embodiments, the helical configuration creates discrete peaks and valleys that minimize contact area with surrounding anatomical structures and catheter lumens, enhancing trackability and directional control. The guidewire may include variable pitch sections, polymer jackets, and hydrophilic coatings to further improve lubricity. In some embodiments, a method includes using the guidewire in combination with one or more catheters having textured inner surfaces to synergistically reduce drag and facilitate navigation through tortuous anatomy. In some embodiments, the system includes a diagnostic catheter that can be delivered to a tortuous pathway simultaneously with the catheter without the need for additional support catheters.

### CROSS-REFERENCE TO RELATED APPLICATIONS

This application claims the benefit of and priority to U.S. Provisional Application No. 63/705,288, filed Oct. 9, 2024, which is hereby incorporated herein by reference in its entirety for all purposes.

### BACKGROUND

Conventional guidewires are commonly used in medical procedures to direct catheters and other devices to specific locations within a patient's body, such as the cardiovascular or neurovascular systems. It is important for the guidewire to navigate the complex pathways of the vasculature with minimal resistance. However, conventional guidewires frequently encounter high friction when in contact with the walls of the vasculature or other devices. This friction impedes movement and decreases the efficiency of torque transmission, making it harder for operators to precisely control the guidewire tip.

Therefore, there is a need in the art for a novel guidewire that can provide decreased friction while allowing for improved torque transmission.

### SUMMARY

In some embodiments, the present disclosure is directed to a system featuring a square profile guidewire suitable for medical applications. In some embodiments, the square profile guidewire (also referred to herein as a “guidewire”) includes a helical twist. In some embodiments, the helical twist includes a pitch ranging from 0.0100 to 0.7500 inches. In some embodiments, the unique shape and pitch of the wire are configured to minimize the contact area between the system and surrounding anatomical structures, or other devices, thereby reducing friction and enhancing operator control, as further described herein.

In some embodiments, the system does not include a wire or tube that has been cut, or includes multiple cuts along a non-longitudinal axis (e.g., laser cut hypotube) that are configured to impart flexibility. In some embodiments, the guidewire includes a solid core, multi-edge (e.g., square or rectangular as non-limiting examples) profiled guidewire twisted and/or formed with a defined pitch between edges to obtain desired flexibility and torque. In some embodiments, the system the twist, also referred to herein as a helix although the twist is not necessarily a true helix in some embodiments, is configured to provide improved navigation within a patient's anatomy and greater control during medical procedures.

In some embodiments, the system includes a core constructed from a square profiled wire that is twisted to achieve a pitch ranging from 0.0100 to 0.7500 inches. In some embodiments, the resulting helical structure is configured to reduce the contact surface area between the system and the surrounding anatomy or interventional devices, thereby minimizing friction and enhancing torque transmission. In some embodiments, the guidewire is configured such that the edges of the square profile receive a greater contact force (or all) than a center portion of the guidewire surface when in a twisted and/or helical configuration. As a result of reducing the contact area of the guidewire to the square profile edges, torque can be transmitted more efficiently by the operator, improving precision and ease of movement, particularly when navigating tight or tortuous pathways within the body, according to some embodiments.

In some embodiments, one or more edges of the guidewire includes a radius of curvature between 0.0010 and 0.0200 inches (in). In some embodiments, one or more edges of the guidewire includes a radius of curvature of 0.0075 in. In some embodiments, one or more sections of the guidewire include a diagonal edge-to-edge diameter (outer diameter (OD)) of 0.0042 to 0.0919 in. In some embodiments, one or more sections of the guidewire include a diagonal edge-to-edge diameter of 0.0238 in. In some embodiments, the guidewire includes a diameter between parallel surfaces (width/thickness) between 0.0030 and 0.0650 in. In some embodiments, the guidewire includes a diameter between parallel surfaces of 0.0191 in.

In some embodiments, the multi-edge profiled wire may be made from materials such as stainless steel, nitinol, or other elastic materials, which are configured to provide the necessary strength and flexibility for medical applications. In some embodiments, according to a method of manufacture, the multi-edge wire is twisted to achieve a defined pitch between 0.0100 to 0.7500 inches. In some embodiments, the multi-edge wire is cast to achieve a defined pitch between 0.0100 to 0.7500 inches. In some embodiments, the system includes a solid core with a square profile and/or a rectangular profile. The multi-edge wire's profile may be twisted along its length to form a helical structure with a pitch ranging from approximately 0.0100 to 0.7500 inches, where the pitch refers to the distance between two consecutive twists of the wire, in accordance with some embodiments.

In some embodiments, the multi-edge profile of the wire is configured to minimize the contact area compared to round-profiled systems by creating valleys in-between peaks of the pitch when viewed from a side profile. By twisting the square-profiled wire into a defined pitch, in accordance with some embodiments, frictional forces along the system's length are reduced, resulting in easier movement through vasculature and improved torque transmission to the distal tip. The reduced frictional contact also minimizes the risk of damage to sensitive anatomical structures.

In some embodiments, the system may be formed from materials such as stainless steel, nitinol, and/or other metals with suitable elasticity and strength to allow the square profile guidewire to bend and conform to a part of an anatomy without permanent deformation. In some embodiments, nitinol has been found to be a suitable choice of material, according to some embodiments, because of its superelastic properties.

In some embodiments, the system's reduced frictional contact area is configured to allow for more efficient transmission of torque from the proximal end to the distal tip. This enables the operator to apply rotational forces with greater precision when navigating through tortuous or highly curved anatomical pathways. In some embodiments, the multi-edge profile wire's shape ensures that torsional forces are evenly distributed along its length, improving the operator's ability to control the tip in real time.

In some embodiments, the pitch of the multi-edge profiled wire can range between 0.0100 to 0.7500 inches, depending on the specific needs of the medical procedure. A tighter pitch, such as 0.0100 inches, may be preferred for highly tortuous pathways, while a loose pitch, such as 0.7500 inches, may be optimal for more straightforward navigation, according to some embodiments.

In some embodiments, a single continuous guidewire includes a multiple pitch helical section within an intermediate section, where each pitch section includes a different pitch than adjacent sections. In some embodiments, the different pitch sections are configured to impart different flexibility characteristics to the guidewire.

In some embodiments, a continuous guidewire may include one or more thickness sections, where each thickness section includes a different thickness than adjacent sections, where thickness refers to a reduction in cross-section in at least one direction. In some embodiments, this further enables specific flexibility and torquability characteristics to be imparted onto the guidewire. In some embodiments, the system includes a polymer coating surrounding at least a portion of the core to further enhance performance.

### DRAWING DESCRIPTION

The features and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 illustrates a guidewire including an elongated core extending from a proximal end to a distal tip, in accordance with some embodiments of the present disclosure;

FIGS. 2A and 2B illustrate two examples of core profiles for the guidewire, including a square profile and a rectangular profile, in accordance with some embodiments of the present disclosure;

FIG. 3 shows two sections of a guidewire core, each configured with a distinct helical pitch, in accordance with some embodiments of the present disclosure;

FIG. 4 depicts a close-up view of a guidewire core edge showing a rounded edge formed at the intersection of adjacent faces of a multi-edge profile, in accordance with some embodiments of the present disclosure;

FIG. 5 illustrates a longitudinal schematic of a guidewire subdivided into multiple sections along its length, in accordance with some embodiments of the present disclosure;

FIG. 6 illustrates a grinder configured for deburring and rounding the proximal tip of a guidewire core, in accordance with some embodiments of the present disclosure;

FIG. 7 depicts a visual inspection of the proximal tip of the guidewire core, comparing an unacceptable sharp tip and an acceptable rounded tip, in accordance with some embodiments of the present disclosure;

FIG. 8 illustrates a surface preparation process for a ground wire sub-assembly, showing a roughened region after sanding, in accordance with some embodiments of the present disclosure;

FIG. 9 illustrates the distal tip of a ground wire sub-assembly positioned within a shape set fixture for shaping, in accordance with some embodiments of the present disclosure;

FIG. 10 illustrates a step of securing the ground wire sub-assembly within the shape set fixture for tip shaping, in accordance with some embodiments of the present disclosure;

FIG. 11 illustrates a bath assembly for heat-setting the distal tip of the guidewire using a fluidized temperature bath, in accordance with some embodiments of the present disclosure;

FIGS. 12A and 12B illustrate verification of sand level within the bath assembly for heat-setting, in accordance with some embodiments of the present disclosure;

FIG. 13 illustrates the process of preparing the shape set fixture for heat-setting, in accordance with some embodiments of the present disclosure;

FIG. 14 illustrates insertion of the loaded shape set fixture into a fluidized temperature bath for heat-setting the distal tip of the guidewire, in accordance with some embodiments of the present disclosure;

FIG. 15 illustrates a cleaning process for a guidewire assembly, in accordance with some embodiments of the present disclosure;

FIG. 16 illustrates loading a polymer tubing segment onto the distal end of the guidewire, in accordance with some embodiments of the present disclosure;

FIG. 17 illustrates cutting and loading a fluorinated ethylene propylene (FEP) heat shrink over the proximal end of the extrusion, in accordance with some embodiments of the present disclosure;

FIG. 18 illustrates applying heat to reflow the proximal end of a polymer tubing segment, in accordance with some embodiments of the present disclosure;

FIG. 19 illustrates peeling the FEP heat shrink from the guidewire after reflow, in accordance with some embodiments of the present disclosure;

FIG. 20 illustrates applying heat to polymer joints during guidewire assembly, in accordance with some embodiments of the present disclosure;

FIG. 21 illustrates verifying and finishing polymer joints after reflow, in accordance with some embodiments of the present disclosure;

FIG. 22 illustrates applying a distal FEP heat shrink during guidewire assembly, in accordance with some embodiments of the present disclosure;

FIG. 23 illustrates securing a portion of a reflow weight fixture to the distal end of the guidewire, in accordance with some embodiments of the present disclosure;

FIG. 24 illustrates configuring a reflow machine for polymer bonding, in accordance with some embodiments of the present disclosure;

FIG. 25 illustrates positioning the guidewire assembly within the reflow tower, in accordance with some embodiments of the present disclosure;

FIG. 26 illustrates attaching reflow weights to the guidewire assembly, in accordance with some embodiments of the present disclosure;

FIG. 27 illustrates closing the funnels around the guidewire assembly within the reflow tower, in accordance with some embodiments of the present disclosure;

FIG. 28 illustrates cutting the proximal end of the polymer extrusions evenly, in accordance with some embodiments of the present disclosure;

FIG. 29 illustrates preparing a heat-shrink segment for proximal tipping, in accordance with some embodiments of the present disclosure;

FIG. 30 illustrates applying heat to shrink a FEP segment over the proximal end of the polymer extrusions, in accordance with some embodiments of the present disclosure;

FIG. 31 illustrates trimming the distal tip of the guidewire using a shape cut fixture, in accordance with some embodiments of the present disclosure;

FIGS. 32A and 32B illustrate a properly centered guidewire tip and an improperly centered tip within surrounding polymer after cutting, in accordance with some embodiments of the present disclosure;

FIG. 33 illustrates applying a heat-shrink segment to encapsulate the distal tip of the guidewire, in accordance with some embodiments of the present disclosure;

FIG. 34 illustrates positioning the encapsulated distal tip within a tip curving fixture, in accordance with some embodiments of the present disclosure;

FIG. 35 illustrates applying heat to the distal tip during the tipping operation, in accordance with some embodiments of the present disclosure;

FIG. 36 illustrates the packaging process for the guidewire, in accordance with some embodiments of the present disclosure;

FIG. 37 illustrates a perspective view of a catheter system including an outer catheter and an inner catheter, in accordance with some embodiments of the present disclosure;

FIGS. 38A and 38B illustrate additional views of the catheter system including hubs and distal ends, in accordance with some embodiments of the present disclosure;

FIGS. 39 and 40 illustrate cross-sectional views of the outer catheter and inner catheter, respectively, in accordance with some embodiments of the present disclosure;

FIG. 41 illustrates a cross-section of the catheter system including both the outer and inner catheters and associated hydrophilic coatings, in accordance with some embodiments of the present disclosure;

FIG. 42 illustrates the outer catheter including a device wall and reinforcement structure, in accordance with some embodiments of the present disclosure;

FIG. 43 illustrates the inner catheter including a device wall and reinforcement structure, in accordance with some embodiments of the present disclosure;

FIG. 44 illustrates another view of the outer catheter showing layers of materials including hydrophilic coatings and reinforcement structures, in accordance with some embodiments of the present disclosure;

FIG. 45 illustrates a catheter system comprising an outer catheter and an inner catheter, in accordance with some embodiments of the present disclosure;

FIG. 46-48 illustrate the inner catheter including a hypotube, coil structure, and braid structure, in accordance with some embodiments of the present disclosure;

FIGS. 49A and 49B illustrate cross-sectional views of a hypotube with heat-shrink material and reflown polymer, in accordance with some embodiments of the present disclosure;

FIG. 50 illustrates another catheter system comprising an outer catheter and an inner catheter, in accordance with some embodiments of the present disclosure;

FIG. 51 illustrates the inner catheter in more detail, including proximal and distal ends, in accordance with some embodiments of the present disclosure;

FIGS. 52A and 52B illustrate cross-sectional views of the hypotube of the inner catheter, in accordance with some embodiments of the present disclosure;

FIG. 53 illustrates a flowchart of a process for coating and curing an inner surface of a catheter, in accordance with some embodiments of the present disclosure;

FIG. 54 illustrates a flowchart of an alternative process for coating and curing an inner surface of a catheter, in accordance with some embodiments of the present disclosure;

FIG. 55 shows results for a three-point bending test comparing conventional catheters and system catheters, in accordance with some embodiments of the present disclosure;

FIG. 56 illustrates a catheter kink test for a system versus a conventional catheter, in accordance with some embodiments of the present disclosure;

FIG. 57 shows data for inner diameter lubricity in a simulated anatomy model, in accordance with some embodiments of the present disclosure;

FIG. 58 shows a measured catheter distal looped compression force, in accordance with some embodiments of the present disclosure;

FIG. 59 shows a cross-sectional view of a catheter shaft including reinforcement structures and hydrophilic coatings, in accordance with some embodiments of the present disclosure;

FIGS. 60A and 60B, show a diagnostic catheter configured for intravascular navigation and imaging.

FIGS. 60C and 60D illustrate the proximal end of the diagnostic catheter.

FIG. 61 illustrates a conventional femoral access procedure that involves multiple device exchanges and incremental steps to achieve adequate support and navigation, in accordance with some embodiment;

FIG. 62 shows the system advancing through a tortuous pathway, in accordance with some embodiments; and

FIG. 63 includes non-limiting method steps for a femoral or radial access procedure, in accordance with some embodiments.

### DETAILED DESCRIPTION

The present disclosure is directed to a guidewire configured for body lumen (e.g., intravascular) navigation during medical procedures. In some embodiments, the guidewire includes an elongated core extending from a proximal end to a distal end, and is structured to provide a balance of torque transmission, flexibility, and atraumatic navigation. In some embodiments, the core includes multiple functional sections, each configured to achieve specific performance characteristics.

In some embodiments, the proximal section includes a cylindrical profile that provides stiffness and pushability, enabling controlled advancement through complex anatomical pathways. In some embodiments, the proximal section includes a helical profile. In some embodiments, the proximal section includes a cylindrical profile, which causes more friction than a helical profile, where the combination of the two have yielded improved torsional control by localizing friction to desired locations.

In some embodiments, the guidewire includes a helical section formed by twisting a multi-edge profile core along a portion of the multi-edge profile core length. In some embodiments, a multi-edge profile includes between 3 and 5 edges. While not beyond the scope of the disclosure, empirical testing has shown that more than 5 edges result in an increase in friction toward that of a cylindrical profile. A 4-edge profile (e.g., square profile) yields desirable characteristics for manufacturability, friction reduction, and uniform bending for most applications, in accordance with some embodiments.

In some embodiments, a 4-edge profile may include an elongated axis (e.g., rectangular profile). In some embodiments, a rectangular profile is formed from reducing the thickness (e.g., grinding) a square profile width to increase local torsional compliance.

In some embodiments, the proximal section transitions from a cylindrical profile to a helical profile at a first transition. Conventional guidewires only include round profiles, and common belief is that a smooth cylindrical surface provides the least resistance. In contrast, it has been discovered that by twisting a square profile into a helical configuration, discrete peaks and valleys are created that reduce surface contact with surrounding anatomical structures and catheter lumens, minimizing friction and improving torque fidelity. In some embodiments, the helical section includes one or more pitch variations along its length to impart different flexibility and torque characteristics. In some embodiments, the helical section may include material segments and/or layers to enhance bonding and radiopacity. In some embodiments, the material includes a polymer. In some embodiments, the first transition includes a tapered geometry (e.g., reduction in square radius) configured to reduce stiffness and create a smooth gradient for controlled navigation toward the distal end.

In some embodiments, the distal end includes a shaped tip configured for atraumatic entry into target vessels. In some embodiments, the distal end includes a pre-shaped or heat-set curve and may include one or more coatings to reduce friction and improve maneuverability. Additional polymer encapsulation and radiopaque elements may be included near the tip to enhance visibility under imaging modalities.

Together, these sections form a guidewire that combines novel structural profiles with selective coatings and shaping techniques, enabling precise operator control, reduced drag, and improved performance during complex interventional procedures, in accordance with some embodiments.

While the non-limiting example discussed herein is directed to a square profile, any portion of the guidewire system may be applied to any multi-edge profile, in accordance with some embodiments. Subject matter may be embodied in a variety of different forms, as well as combinations of features depicted in non-limiting configurations. Therefore, covered or claimed subject matter is intended to be construed as not being limited to any example configuration of structures or function set forth herein. Example configurations, which borrow from portions of the system as a whole, are provided merely to show how one of ordinary skill would make and use the system using some embodiments of the present disclosure. Likewise, a broad scope for claimed or covered subject matter is intended. Among other things, for example, subject matter may be embodied as methods, devices, components, computer implemented instructions, control systems, and/or structure. The following detailed description is, therefore, not intended to be taken in a limiting sense.

As used throughout the specification and claims, terms may carry nuanced meanings that are informed by context and are not limited to explicitly stated definitions. The phrase “in some embodiments” is not intended to refer exclusively to the same embodiment or to distinct embodiments, unless clearly indicated. Furthermore, the absence of the phrase “in some embodiments” in a sentence should not be interpreted to mean that the described subject matter cannot be combined with or omitted from other elements or embodiments described herein when defining the metes and bounds of the system. Thus, the system may be described using any combination of the features described herein, including those disclosed in relation to structure and manufacturing of the structure.

In general, terminology may be understood at least in part from usage in context. For example, conjunctions such as “and,” “or,” and the phrase “and/or” are, by default, inclusive. When a list such as A, B, or C is joined by “or,” the phrase encompasses any of A, B, or C individually as well as any combination of two or three of them, unless the surrounding text expressly limits the meaning to a single, mutually exclusive option. Likewise, “and” and “and/or” refer to one or more of the listed items in any combination.

The phrase “one or more” indicates that the referenced element may be present as a single instance or as multiple instances and should be understood to cover either possibility unless a different intent is clearly expressed. Similarly, the articles “a,” “an,” and “the” can denote either singular or plural usage depending on context, unless the claim language expressly limits them to one or to multiple instances.

The phrase “based on” is nonexclusive; it signifies that the identified factor is considered in whole or in part and does not preclude reliance on additional, unstated factors unless the claim language explicitly restricts the analysis to the recited factor alone.

As used herein, the verbs “can” and “may,” and derivations thereof, denote functional capability, where a clause such as “the system can execute instructions X” is equivalent in scope to “the system is configured to execute instructions X” when defining the metes and bounds of the claimed subject matter. The phrase “configured to” indicates that the identified structure or computer has been arranged, programmed, or otherwise adapted to perform the recited operation. The terms “can” and “may” also highlight the modular nature of the architecture, signifying that individual components may be present, omitted, or combined in different implementations without departing from the scope of the claims, in a manner analogous to phrases such as “in some embodiments,” “according to some embodiments,” “in accordance with some embodiments,” or comparable expressions.

As used herein, “guidewire” refers to an elongated, flexible medical device configured to navigate through anatomical pathways and provide a track for catheters or other interventional instruments, in accordance with some embodiments.

As used herein, “core” refers to the primary structural element of the guidewire which provides torque transmission, pushability, and structural integrity. In some embodiments, a “core” may be formed by joining together one or more sections described herein.

A “square profile” refers to a cross-sectional geometry of the core having four sides and one or more radiused edges, in accordance with some embodiments.

A “helical structure” refers to a twisted configuration of the core in which the square profile is rotated about its longitudinal axis to form a spiral pattern defining a pitch between successive turns. The pitch of a helical structure may vary along its length, in accordance with some embodiments.

As used herein, “pitch” refers to the axial distance between corresponding points on adjacent turns of the twisted core structure.

As used herein, “proximal section” refers to the portion of the guidewire nearest the operator, generally configured to provide pushability and torque input, in accordance with some embodiments.

As used herein, “intermediate section” refers to the portion of the guidewire between the proximal and distal sections. In some embodiments, the intermediate section includes a twisted square profile configured to enhance torque transmission and flexibility.

As used herein, “distal section” refers to the portion of the guidewire furthest from the operator, configured for flexibility, atraumatic navigation, and radiopacity, in accordance with some embodiments.

As used herein, “radiopaque element” refers to a component or material integrated into the guidewire, such as a coil or polymer sleeve including radiopaque fillers, configured to enable visualization under imaging modalities, in accordance with some embodiments.

As used herein, “polymer jacket” refers to an outer layer of polymer material applied over at least a portion of the core to improve lubricity and provide a smooth interface with surrounding anatomy, in accordance with some embodiments.

As used herein, “lubricious coating” refers to a surface treatment, such as a hydrophilic polymer coating, applied to reduce friction during advancement and withdrawal of the guidewire, in accordance with some embodiments.

As used herein, “shapeable tip” refers to a distal tip portion that can be manually bent or pre-shaped to facilitate navigation into branch vessels, in accordance with some embodiments.

As used herein, “atraumatic tip” refers to a distal end configuration configured to minimize trauma to vessel walls, which may include polymer encapsulation or rounded geometry, in accordance with some embodiments.

As used herein, “variable pitch section” refers to a segment of the twisted core where the pitch changes along its length to impart different flexibility or torque characteristics.

As used herein, “normalized contact perimeter” refers to a measure of the effective contact length between the guidewire and an adjacent surface, normalized to a reference geometry, used to quantify friction reduction.

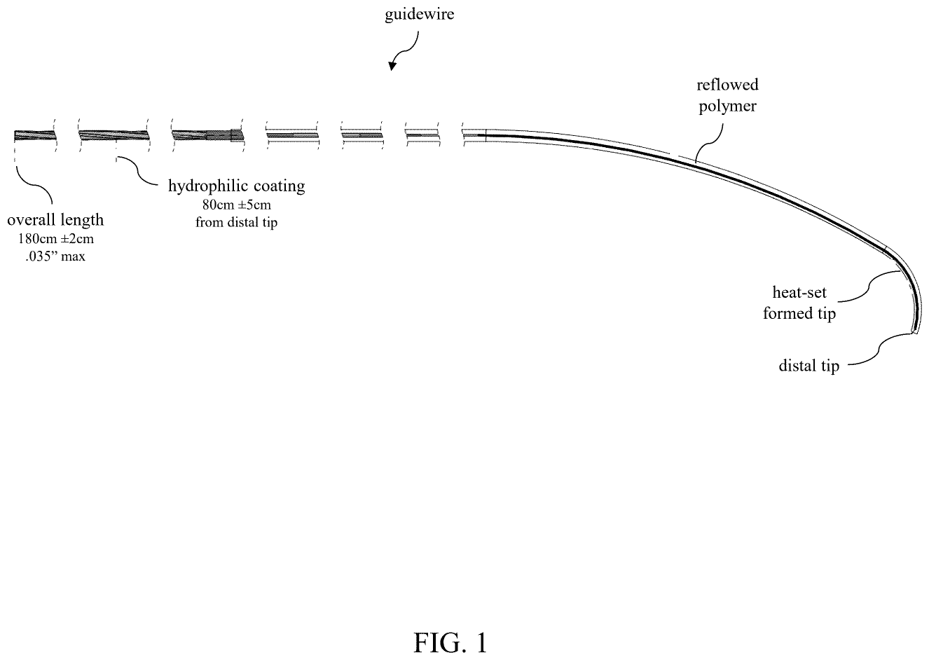

FIG. 1 illustrates a guidewire including an elongated core extending from a proximal end to a distal tip. In this non-limiting example, in accordance with some embodiments, the guidewire includes an overall length of 180 cm±2 cm and a maximum outer diameter of 0.035 inches. In some embodiments, a section of the guidewire includes a hydrophilic coating extending approximately 80 cm±5 cm from the distal tip toward the proximal end. In some embodiments, the distal portion of the guidewire includes a reflowed polymer section, followed by a heat-set formed tip, and terminates at the distal tip.

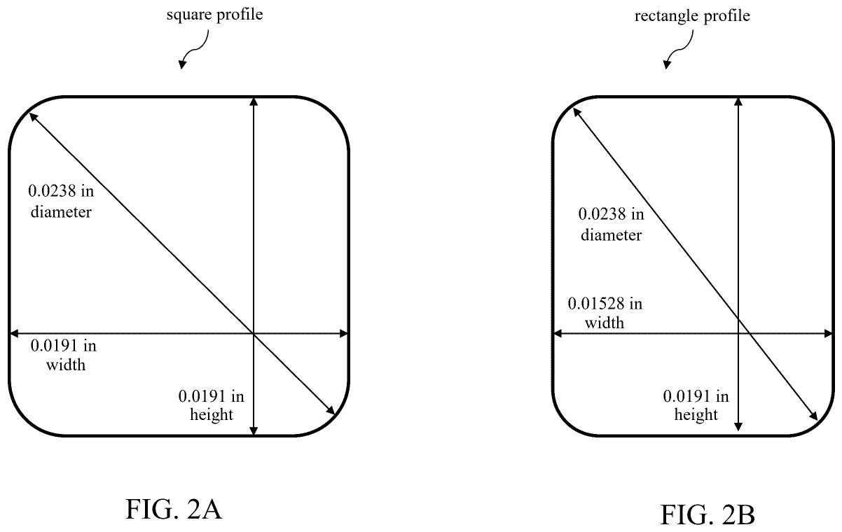

FIGS. 2A and 2B illustrate two examples of core profiles for the guidewire, in accordance with some embodiments. FIG. 2A shows a square profile including four edges with radiused corners, in accordance with some embodiments. In some embodiments, the square profile includes a width and height that are substantially equal, where the diagonal dimension is 0.0238 inches in this non-limiting example. In some embodiments, the width and height are each 0.0191 inches. FIG. 2B shows a rectangular profile including four edges with radiused corners, in accordance with some embodiments. In some embodiments, a rectangular profile includes a height of 0.0191 inches and a width of 0.01528 inches, with a diagonal dimension of 0.0238 inches. Both profiles are configured to provide multi-edge geometry suitable for forming a helical section of the guidewire, in accordance with some embodiments.

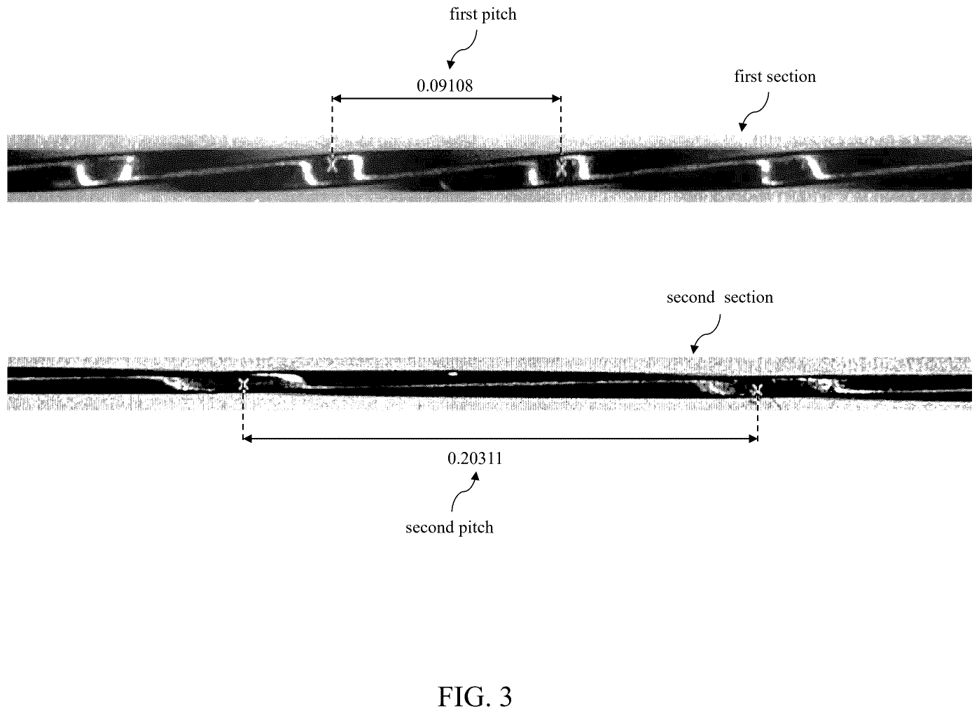

FIG. 3 illustrates two sections of a guidewire core, each configured with a distinct helical pitch, in accordance with some embodiments. In some embodiments, first helical section includes a helical configuration having a first pitch, which is represented by the spacing between consecutive twists along the longitudinal axis. In some embodiments, a second helical section includes a helical configuration having a second pitch, which is greater than the first pitch, providing a different flexibility and torque characteristic. In some embodiments, the second helical section includes a smaller thickness and/or diameter than the first helical section. Some embodiments more helical sections with different or the same pitches as the first and second helical portions.

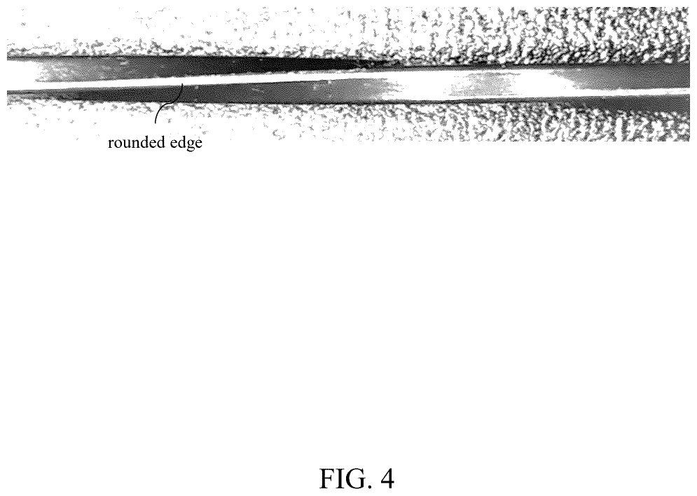

FIG. 4 illustrates a close-up view of a guidewire core edge, showing a rounded edge formed at the intersection of adjacent faces of a multi-edge profile, in accordance with some embodiments. The rounded edge provides a smooth transition between surfaces, reducing sharp corners that could increase friction or stress concentration during navigation. In some embodiments, a suitable radius of curvature for the rounded edge includes a range of approximately 0.0010 inches to 0.0200 inches, providing sufficient rounding to reduce friction while maintaining structural integrity and manufacturability.

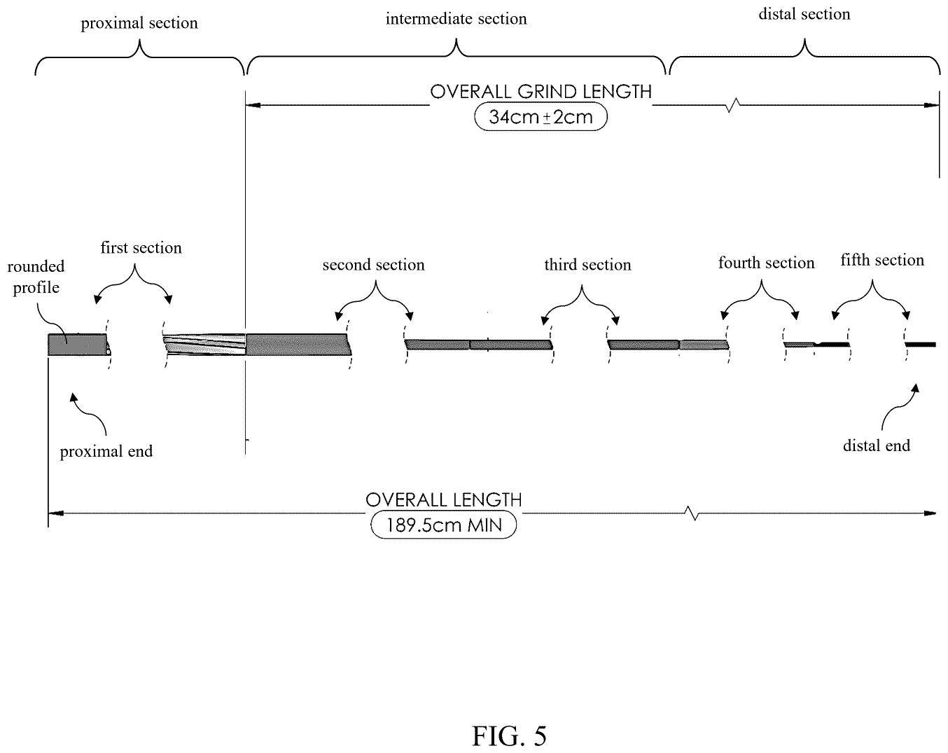

FIG. 5 illustrates a longitudinal schematic of a guidewire extending from a proximal end to a distal end and subdivided into a plurality of sections that are separated by labeled transitions, in accordance with some embodiments. In some embodiments, the sections include, in order from the proximal end to the distal end, a first section (proximal region), a second section (helical region), a third section (transition zone), a fourth section (distal region), and a fifth section (tip). A first transition, a second transition, a third transition, and a fourth transition are disposed between adjacent sections to indicate changes in geometry, surface treatment, and functional intent along the longitudinal axis.

In some embodiments, the guidewire includes an overall length suitable for intravascular procedures across cardiovascular, neurovascular, and peripheral anatomies. In this non-limiting example, the overall length is 189.5 cm, however, in some embodiments the range of the overall length extends from 90 cm to 350 cm. In some embodiments, the guidewire includes a maximum outer diameter of ≤0.035 in along at least a portion of the shaft. The core geometry includes a solid wire with a square cross-sectional profile that is twisted into a helical structure along at least the second section to reduce surface contact and improve torque transmission when compared to a round profile of similar diameter.

From the core outward, the make-up of the guidewire depicted in FIG. 5 includes one or more layers that may be present singly or in combination, depending on section.

In some embodiments, the core wire includes one or more of stainless steel, nitinol, or a cobalt alloy, selected to provide an appropriate balance of stiffness, elasticity, and fatigue resistance, in accordance with some embodiments. In some embodiments, different materials are joined at transition points to form a continuous structure. In some embodiments, the core geometry includes a square profile with radiused edges, where one or more edges include a radius of curvature within 0.0010-0.0200 in, a diagonal edge-to-edge dimension within 0.0042-0.0919 in, and a parallel face width within 0.0030-0.0650 in. In some embodiments, along the helical region, the square profile is twisted with a helical pitch within 0.002-1.500 in (with a suitable range of 0.0100-0.7500 in), and a twisted portion length of ≥15 cm. In some embodiments, multiple pitch zones are included to tune flexibility and torque response.

In some embodiments, a polymer jacket is disposed over one or more sections of the core. The jacket may include Neusoft polymer segments that may include tungsten-loaded sections for radiopacity and are joined by reflow for continuous coverage in the intermediate and distal portions. In some embodiments, at least part of the proximal section is left without a jacket to facilitate pushability.

In some embodiments, a surface coating is applied, such as a hydrophilic polymer coating (HPC) or polytetrafluoroethylene (PTFE), with >90% coverage across the coated length. In the non-limiting example depicted in FIG. 5, the coated length extends approximately 80 cm from the distal tip.

Radiopaque elements may be provided in the distal region, which may include a platinum or tungsten coil or a polymer sleeve loaded with barium or tungsten fillers, to enhance fluoroscopic visibility, in accordance with some embodiments.

In some embodiments, a tip encapsulation is provided at the distal extremity, which may include a polymer material configured to present a smooth distal end and prevent exposure of the core, promoting atraumatic performance.

Sill referring to FIG. 5, the guidewire includes three primary structural sections arranged along a longitudinal axis from a proximal end to a distal tip, in accordance with some embodiments. In some embodiments, the proximal section includes a cylindrical profile configured to provide column strength and torque input for controlled advancement through anatomical pathways. In some embodiments, the proximal section includes a full outer diameter suitable for pushability, where the surface may remain uncoated or partially coated to maintain torque fidelity. The proximal section may be helical or round, depending on desired characteristics, in accordance with some embodiments.

Adjacent to the proximal section, an intermediate section may include a twisted square-profile core forming a helical structure. In some embodiments, the helical configuration creates discrete peaks and valleys that reduce surface contact with catheter lumens, thereby minimizing friction and improving torque transmission. In some embodiments, the intermediate section includes one or more variable pitch zones to impart different flexibility characteristics, and may include one or more polymer jacket segments to enhance lubricity and radiopacity.

In some embodiments, the intermediate section and/or distal section include a tapered geometry, which may be formed by grinding or shaping the core to a reduced diameter, creating a stiffness gradient that facilitates controlled navigation toward the distal end. In some embodiments, the distal tip includes a heat-set or pre-shaped curve configured for atraumatic entry into target vessels. In some embodiments, the distal section includes polymer encapsulation and one or more radiopaque elements, such as a coil or tungsten-loaded polymer sleeve, to enable visualization under imaging modalities. A hydrophilic coating may extend over at least a portion of the distal section to reduce friction and improve trackability.

In some embodiments, the guidewire includes a first section extending from the proximal end along a substantial portion of the overall length. In some embodiments, the first section includes a cylindrical core profile configured to provide column strength and torque input for controlled advancement through anatomical pathways. In some embodiments, the first section occupies the majority of the guidewire length, such as the first 100 to 150 centimeters of a guidewire having an overall length of approximately 190 centimeters. In some embodiments, the proximal section includes a full outer diameter suitable for pushability, for example up to 0.035 inches, and may be coated, or may remain uncoated or partially coated to maintain torque fidelity. In some embodiments, the proximal section includes a portion that is helical to localize friction and enhance torsional control, while the remaining portion includes an untwisted cylindrical profile to maximize stiffness. In some embodiments, the first section is configured to transmit rotational forces applied by the operator with minimal loss, enabling precise manipulation of the distal tip during navigation through tortuous anatomical pathways.

In some embodiments, the guidewire includes a second section that comprises a helical region formed by twisting a square-profile core along a portion of its length. In some embodiments, the second section includes a multi-edge geometry configured to define discrete peaks and valleys when viewed along the longitudinal axis, reducing surface contact with surrounding anatomical structures and catheter lumens by as much as 50%. The helical region may include a diagonal edge-to-edge dimension and a width between parallel faces that are selected to maintain structural integrity while minimizing frictional engagement. In some embodiments, the helical region includes a pitch that may be constant or variable along its length, where variable pitch sections are configured to impart different flexibility and torque characteristics to the guidewire. In some embodiments, the helical region may include one or more polymer segments disposed along its surface. In some embodiments, the one or more polymer segments may include materials configured to enhance radiopacity, enabling improved visualization under imaging modalities.

In some embodiments, the guidewire includes a third section that comprises a transition zone disposed between the helical region and the distal portion of the core. The transition zone may include a cylindrical taper, which may be formed by grinding or shaping the core to reduce its diameter relative to the proximal section. This reduction in diameter is configured to impart increased flexibility while maintaining sufficient structural integrity for torque transmission. In some embodiments, the third section includes one or more polymer segments positioned along its length, such as Neusoft tubing or tungsten-loaded polymer sleeves, which are configured to provide radiopacity and enhance bonding to adjacent sections. In some embodiments, the transition zone creates a stiffness gradient that enables controlled navigation from the relatively rigid proximal section toward the highly flexible distal tip, improving maneuverability and reducing the risk of vessel trauma during advancement, in accordance with some embodiments.

In some embodiments, the guidewire includes a fourth section that comprises a distal region extending toward the terminal end of the core. In some embodiments, the distal region includes a curve profile formed by heat-setting the core in a fluidized bath to achieve a pre-shaped or curved configuration suitable for atraumatic vessel entry. In some embodiments, the fourth section includes a reduced diameter relative to the first section, section, and/or the third section, providing enhanced flexibility for navigating branch vessels and tortuous anatomical pathways. In some embodiments, the fourth section includes a hydrophilic coating applied beginning at or near the distal tip, which is configured to reduce friction and improve trackability during advancement and withdrawal. In some embodiments, the fourth section may include polymer encapsulation and radiopaque elements integrated into or adjacent to the tip to enable visualization under imaging modalities.

In some embodiments, the reduction in cross-section from the second to fifth section can range between 40% and 80%, depending on the desired characteristics. In some embodiments, a percentage drop from the reduction in cross-section for each section from the second to fifth section may be between 10% and 50%, with a total reduction in cross-section of the core not exceeding 80%.

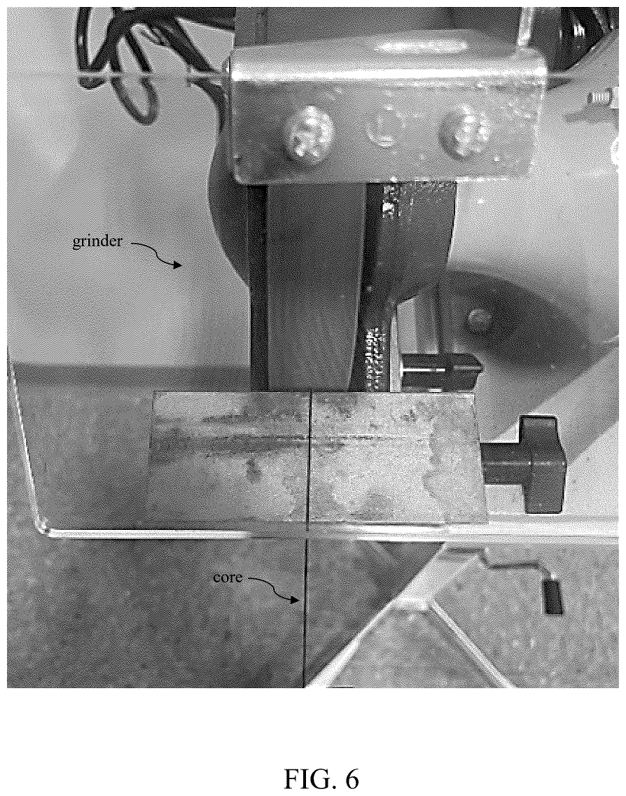

FIG. 6 illustrates a grinder configured for deburring and rounding the proximal tip of a guidewire core. In some embodiments, the manufacturing process includes a step of cutting the ground wire subassembly to a specified length and deburring the proximal tip to achieve a smooth, rounded profile. The process includes measuring 189.5 cm from the distal tip of the ground wire subassembly and cutting the wire at this location using flush cutters to ensure dimensional accuracy. In some embodiments, the pedestal grinder is activated and verified for operational readiness. The grinder wheels are positioned at a distance less than or equal to 1/16 inch from the tool rest, and if the distance exceeds this value, the process is halted until corrective adjustment is made. The wire is then spun while introduced to the rough grinding wheel of the pedestal grinder to remove burrs from the proximal tip. After burr removal, the proximal tip is placed against the soft grinding wheel to form a rounded end, reducing sharp edges that could cause vessel trauma. A visual inspection is performed to confirm that the proximal tip is rounded and free of sharp edges. If the tip remains sharp, the grinding process is repeated. The overall length of the wire from distal tip to proximal end is verified to be 189.5±2 cm, and if the length exceeds tolerance, trimming and deburring are repeated. If the length is below tolerance, the part is removed from production.

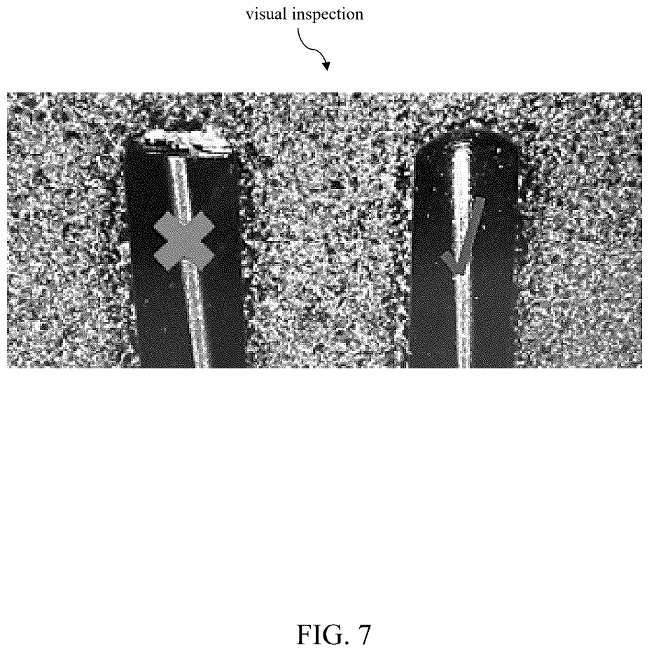

FIG. 7 illustrates a visual inspection of the proximal tip of the guidewire core, showing a comparison between an unacceptable sharp tip and an acceptable rounded tip. In some embodiments, the manufacturing process includes a visual inspection to verify that the proximal tip is rounded and free of burrs.

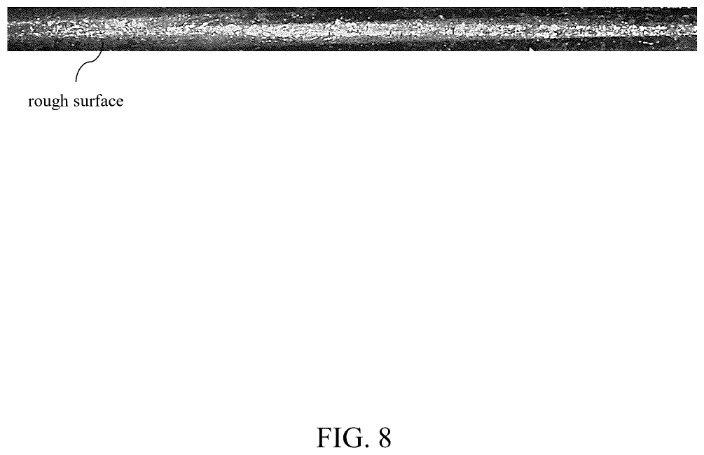

FIG. 8 illustrates a surface preparation process for a ground wire sub-assembly, showing the region that has been roughened through sanding. The figure depicts the wire after sanding with a visibly textured surface, indicating removal of smooth finish to promote adhesion in subsequent coating steps. The process involves marking a location approximately 7 cm proximal to a defined transition point and sanding a segment approximately 4 cm distal of that mark using sandpaper and a sanding stone. The process is verified through visual inspection to ensure uniform surface preparation before advancing to the next manufacturing stage.

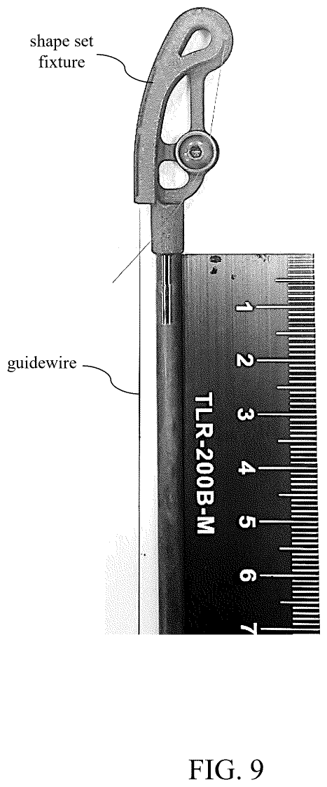

FIG. 9 illustrates the distal tip of a ground wire sub-assembly positioned within a shape set fixture for shaping, in accordance with some embodiments. In some embodiments, the process includes aligning the distal transition marking approximately 0.5 cm from the base of the fixture, wrapping the distal tip along the curved profile, and securing it under a clamping screw tightened with an Allen wrench.

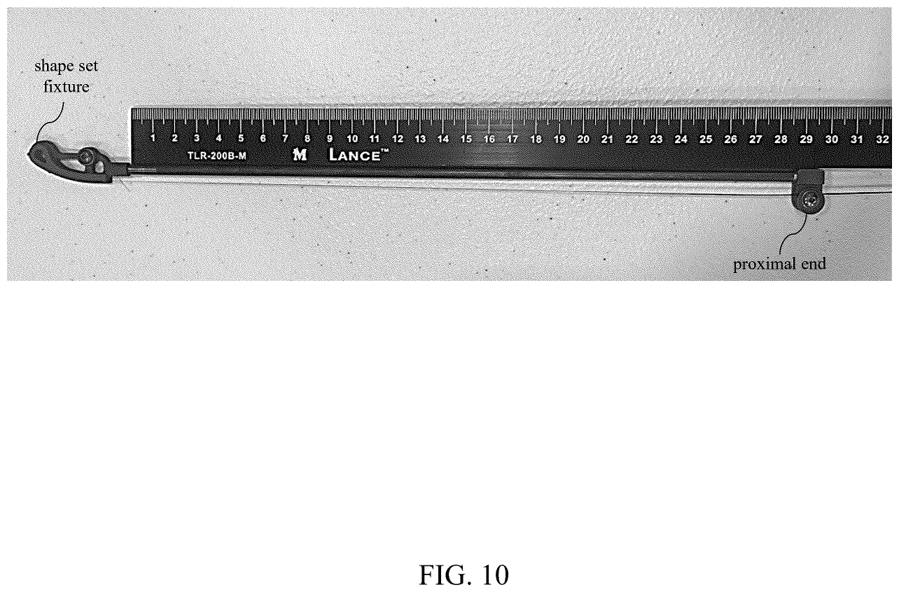

FIG. 10 illustrates a step of securing the ground wire sub-assembly within the shape set fixture for tip shaping. In some embodiments, the process includes tightening the screw located near the proximal end of the fixture to ensure the wire is firmly held in place along the curved profile.

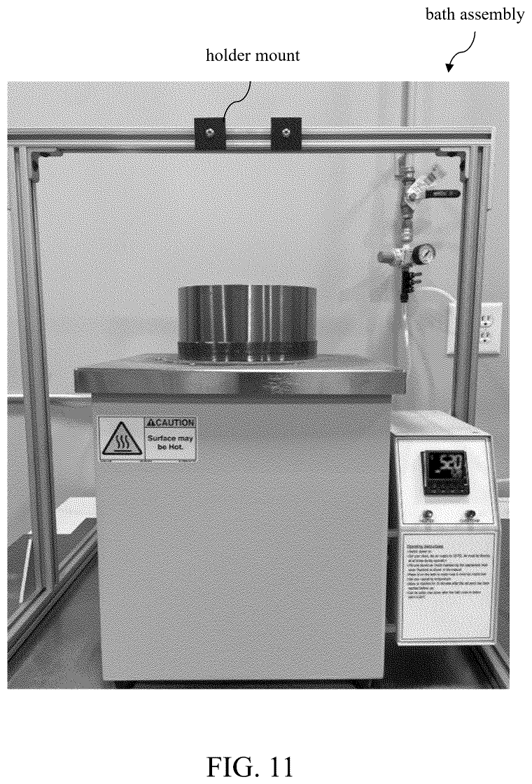

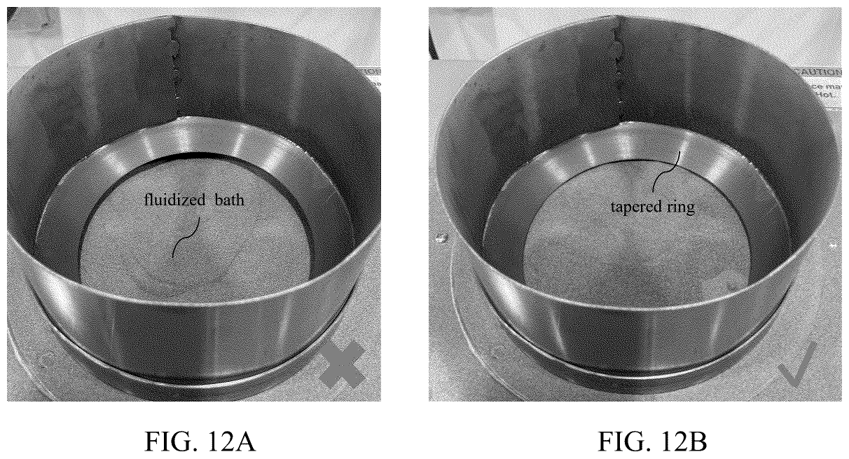

FIG. 11 illustrates a bath assembly for heat-setting the distal tip of the guidewire using a fluidized temperature bath. In some embodiments, a fluidized bath is configured to maintain a temperature of 510±15° C. and a pressure of 30 PSI±10 PSI. As illustrated in FIGS. 12A and 12B a sand (or other suitable material) level is verified to reach at least the tapered ring of the bath assembly.

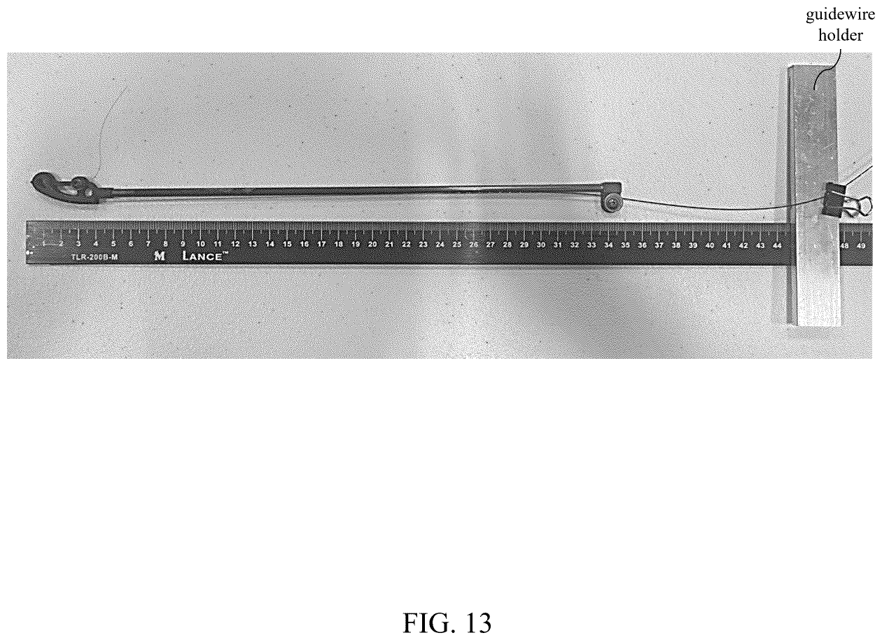

FIG. 13 illustrates the process of preparing the shapes set fixture for heat-setting. The process includes measuring 45 cm from the end of the fixture using a calibrated ruler and securing the guidewire to a guidewire holder component of the bath assembly at the measured location.

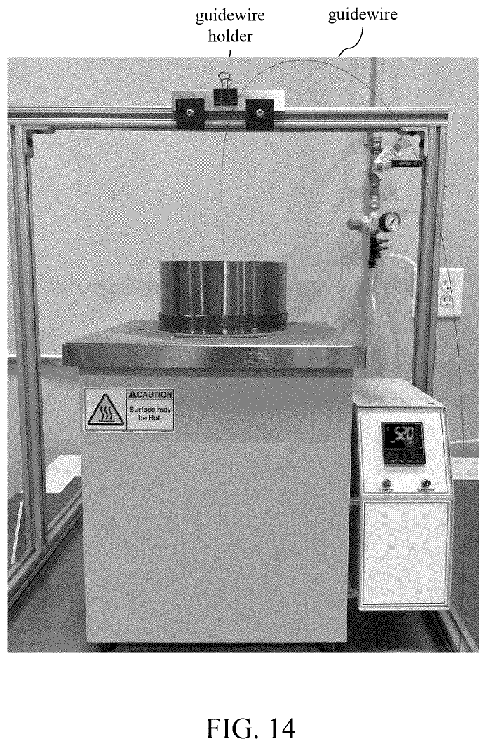

FIG. 14 illustrates the insertion of the loaded shape set fixture into a fluidized temperature bath for heat-setting the distal tip of the guidewire. In some embodiments, the shape set fixture is positioned so that its guidewire holder rests securely on the holder mount above the bath, ensuring proper alignment during the thermal process.

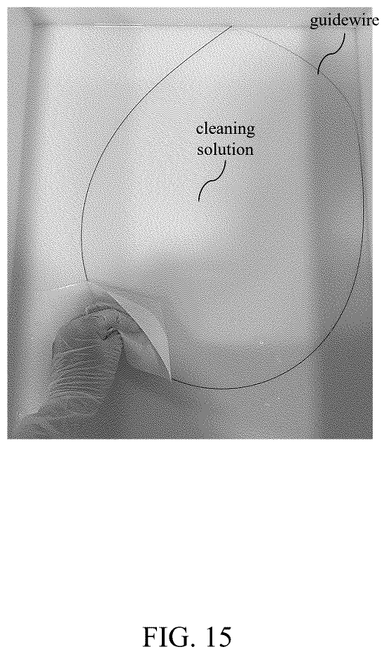

FIG. 15 illustrates a cleaning process for a guidewire assembly, in accordance with some embodiments. In some embodiments, the process includes placing the guidewire into a cleaning solution. In some embodiments, the cleaning solution includes a mixture of one part Liquinox detergent and one hundred parts deionized water, although other suitable solutions may be used. In some embodiments, the process includes gently scrubbing the entire length of the wire with a lint-free wipe wetted in the cleaning solution to remove surface contaminants. In some embodiments, the process includes removing the wire from the detergent solution and placing it into a second cleaning solution. In some embodiments, the second cleaning solution includes deionized water, or other suitable solutions, configured to rinse away residual detergent. In some embodiments, the process includes removing the wire from the second cleaning solution and wiping its entire length, for example with a lint-free wipe wetted in a third cleaning solution, such as seventy percent isopropyl alcohol, and allowing the wire to dry before placing it into a sealed polybag for transfer.

In some embodiments, the manufacturing process includes a polymer reflow process. In some embodiments, the process includes wiping the entire length of the wire using a lint-free wipe wetted with seventy percent isopropyl alcohol to remove surface contaminants. In some embodiments, the process includes obtaining polymer extrusions and cutting them to specified lengths, including segments of 62A Neusoft tubing cut to 1.5±0.5 cm and 5.5±1.0 cm, a segment of 73A Neusoft tubing cut to 2.5±1.0 cm, segments of 62A tungsten-loaded Neusoft tubing cut to 6.5±2.0 cm and 0.50±0.30 cm, and segments of 42A tungsten-loaded Neusoft tubing cut to 1.25±0.50 cm and 7.00±1.00 cm.

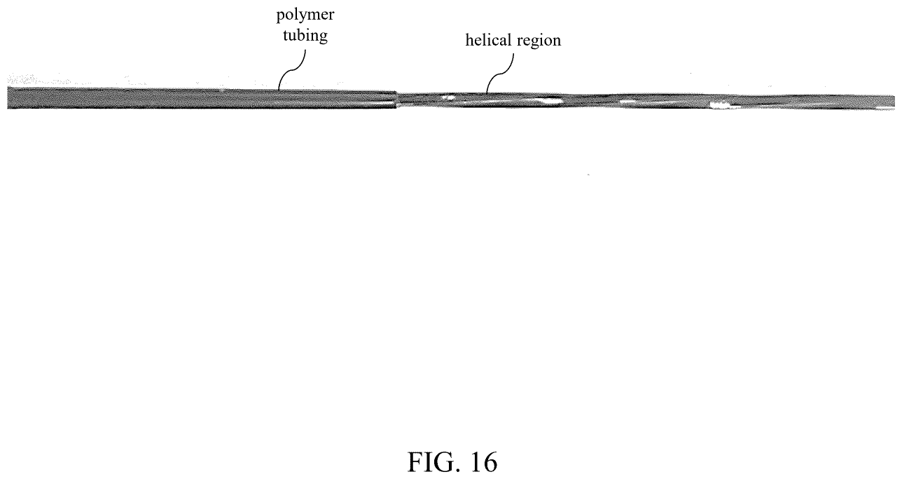

As shown in FIG. 16, in some embodiments, the process includes loading a 1.5 cm segment of a polymer tubing (e.g., 62A Neusoft tubing) from the distal end of the wire until the distal end of the tubing is positioned against the helical region of the wire.

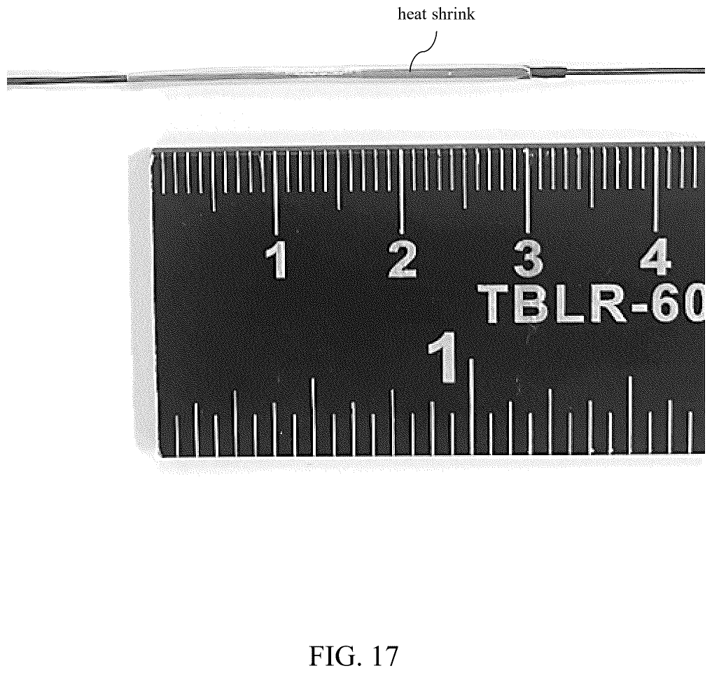

As shown in FIG. 17, in some embodiments, the process includes cutting an approximately 3 cm piece of fluorinated ethylene propylene (FEP) heat shrink with a slit at one end, using an ionizing air gun to clear the piece, and loading and centering the FEP over the proximal end of the extrusion.

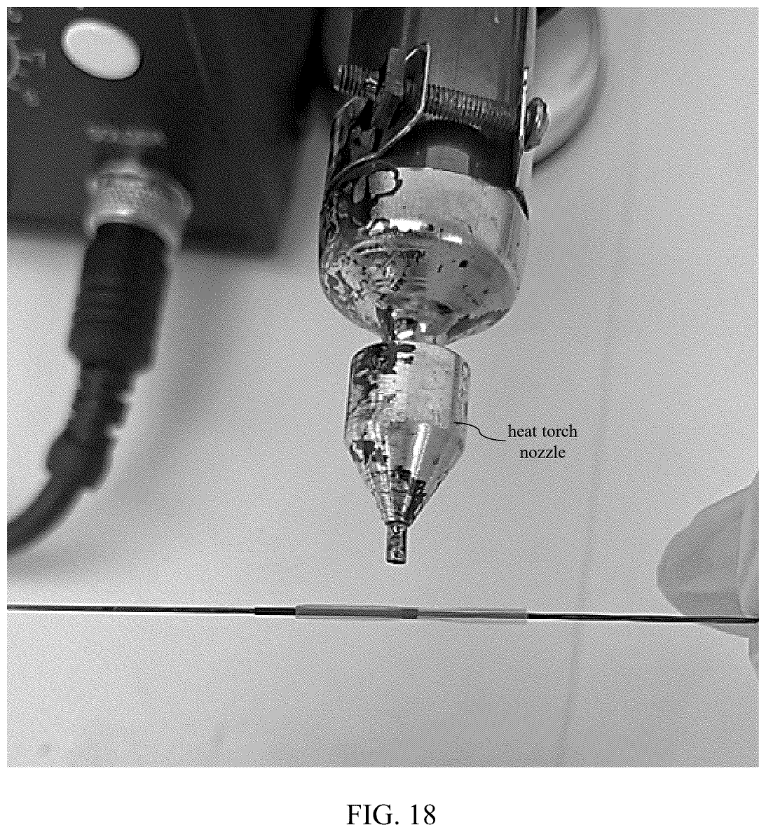

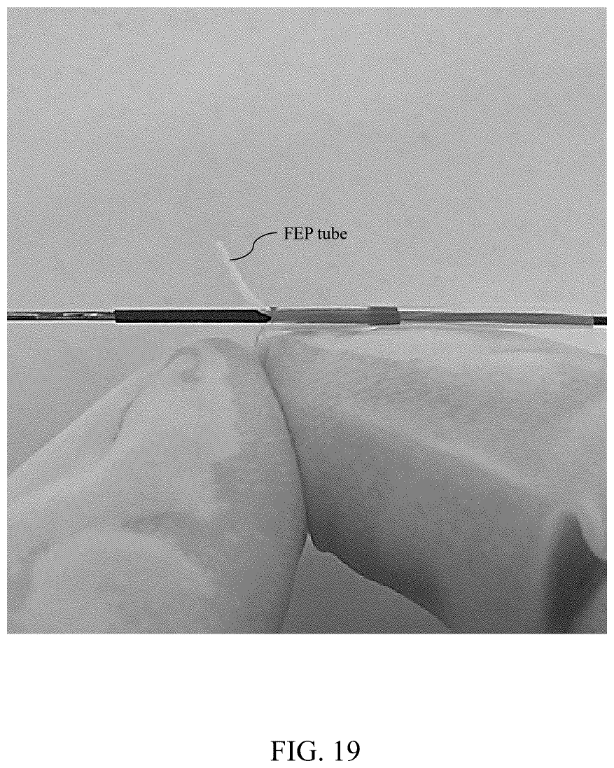

FIG. 18 illustrates a portion of the manufacturing process that includes applying heat using a calibrated heat torch set to 550° F. with a heat torch nozzle to reflow the proximal end of the 62A Neusoft tubing segment, securing it in place. FIG. 19 illustrates gently peeling the FEP until it is removed from the guidewire, in accordance with some embodiments.

In some embodiments, the process includes loading and securing polymer segments during guidewire assembly. In some embodiments, the process includes loading the remaining polymer segments from the distal end of the wire in the following order: a segment of 62A Neusoft tubing measuring 5.5±1.0 cm, followed by a segment of 73A Neusoft tubing measuring 2.5±1.0 cm, a segment of 62A tungsten-loaded Neusoft tubing measuring 6.5±2.0 cm, a segment of 42A tungsten-loaded Neusoft tubing measuring 1.25±0.50 cm, an additional segment of 62A tungsten-loaded Neusoft tubing measuring 0.50±0.30 cm, and a final segment of 42A tungsten-loaded Neusoft tubing measuring 7.00±1.00 cm. In some embodiments, the process includes positioning the 5.5 cm segment of 62A Neusoft tubing approximately 0.5 cm proximal of the marked proximal transition to ensure proper alignment.

In some embodiments, the process includes cutting a 3 cm piece of FEP heat shrink with a slit at one end and loading the FEP over the joint from distal to proximal, covering the interface between adjacent polymer segments. This step is repeated for each joint, ensuring that all joints are aligned and touching under the FEP prior to heat application.

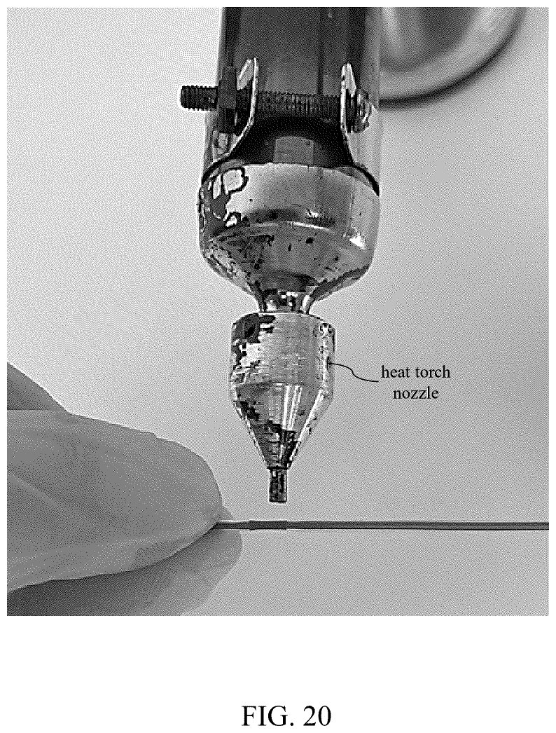

FIG. 20 illustrates applying heat to each joint using a calibrated heat torch set to 550° F. with a heat torch nozzle. The heat is applied while rotating the assembly to ensure uniform bonding of the polymer segments along the wire.

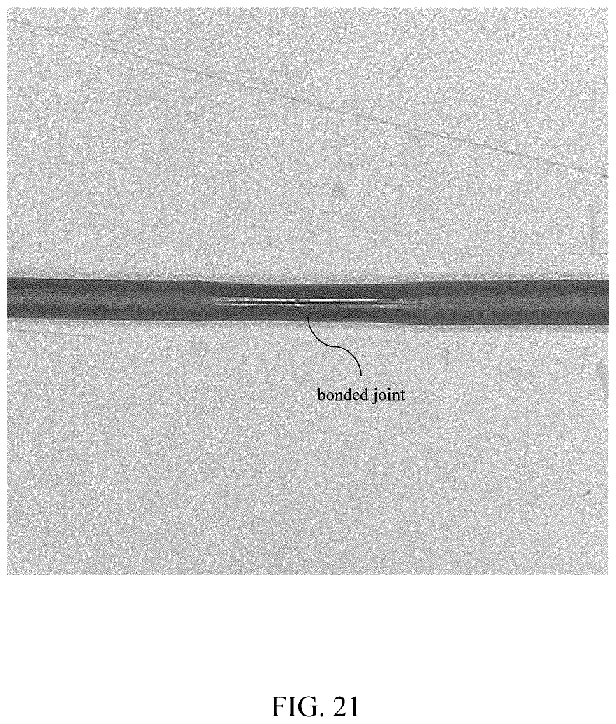

FIG. 21 illustrates verifying and finishing polymer joints. In some embodiments, the process includes ensuring that all joints are fully bonded with no exposed wire along the length of the polymer-coated region. In some embodiments, the process includes removing the FEP heat shrink by tearing along the slit after the reflow operation is complete, leaving a continuous polymer coating around the wire.

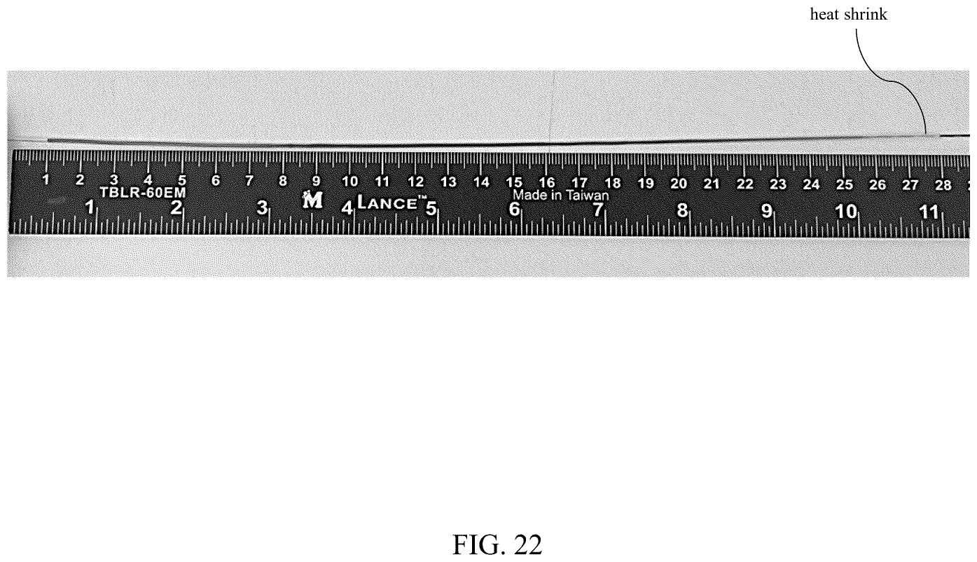

FIG. 22 illustrates a process for applying a distal FEP heat shrink during guidewire assembly, in accordance with some embodiments. In some embodiments, the process includes cutting a piece of FEP heat shrink to an approximate length of 28 cm and using an ionizing air gun to clear the interior of the FEP segment. In some embodiments, the process includes loading the FEP heat shrink over the distal portion of the wire until its proximal end is positioned approximately 2 cm distal of the 7 cm segment of 42A tungsten-loaded Neusoft tubing. In some embodiments, the process includes applying heat to the proximal 2 cm of the FEP using a calibrated heat torch set to 550° F. with a heat torch nozzle, shrinking the FEP securely around the wire to create a smooth transition and maintain alignment of the polymer segments.

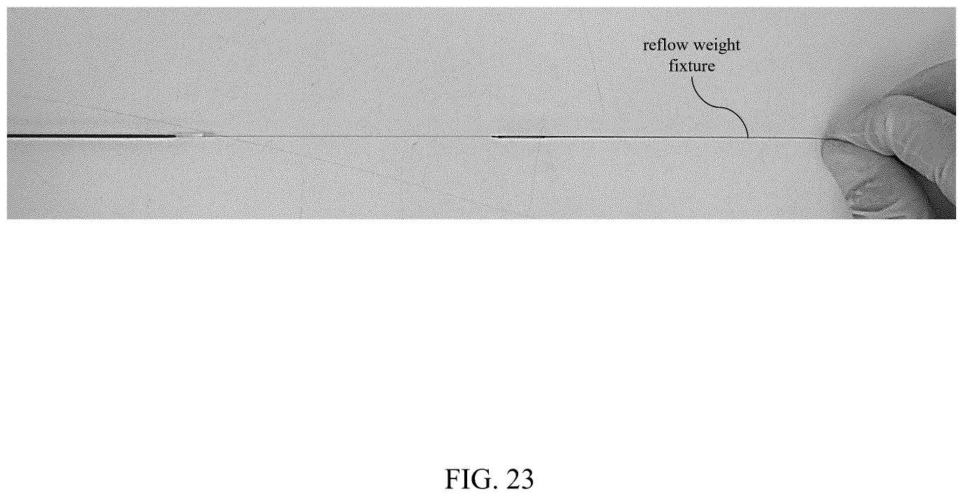

FIG. 23 illustrates the process of securing a portion of a reflow weight fixture to the distal end of the guidewire, in accordance with some embodiments. In some embodiments, the process includes positioning the reflow weight fixture at the distal end of the wire and locking it in place to provide controlled tension during the reflow operation.

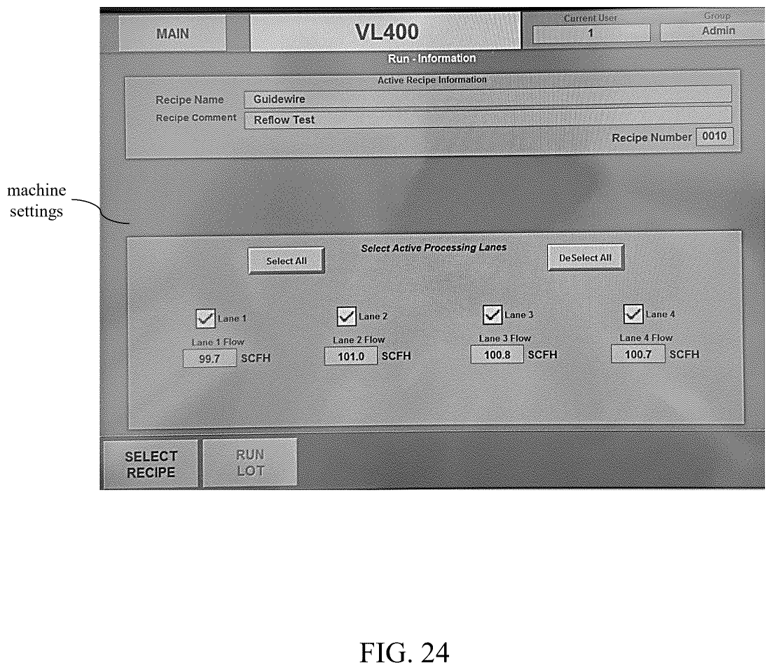

FIG. 24 illustrates the process of configuring a reflow machine for polymer bonding, in accordance with some embodiments. In some embodiments, the process includes turning on the reflow machine and opening a recipe (e.g., Recipe 0010 “Guidewire”) In some embodiments, the process includes verifying that the machine settings are adjusted to a temperature of 420 degrees Fahrenheit±5 degrees Fahrenheit and a flow rate of 100 standard cubic feet per hour (SCFH)±5 SCFH. In some embodiments, the process includes selecting the appropriate lanes for operation and using the control knobs to adjust each lane to meet the specified parameters. In some embodiments, the process includes confirming that the flow has stabilized within the required range before initiating the reflow cycle.

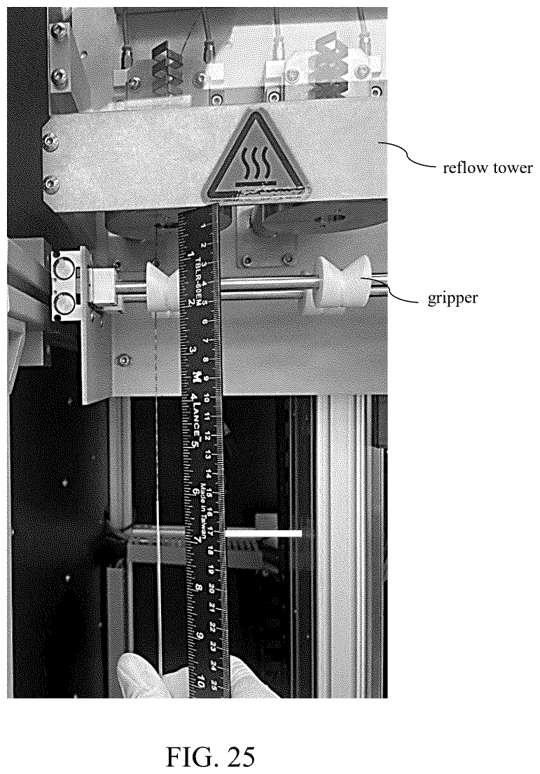

FIG. 25 illustrates the process of positioning the guidewire assembly within the reflow tower, in accordance with some embodiments. In some embodiments, the process includes inserting the proximal end of the wire through the funnels and past the grippers until the start of the proximal polymer segment is aligned approximately 17 cm from the heaters, as indicated by the reference marker. In some embodiments, the process includes pressing the gripper button located above the alignment point so that the indicator light activates and the gripper closes, securing the wire in place for thermal processing. In some embodiments, the process includes repeating this positioning and clamping step for all lanes to ensure uniform setup prior to initiating the reflow cycle.

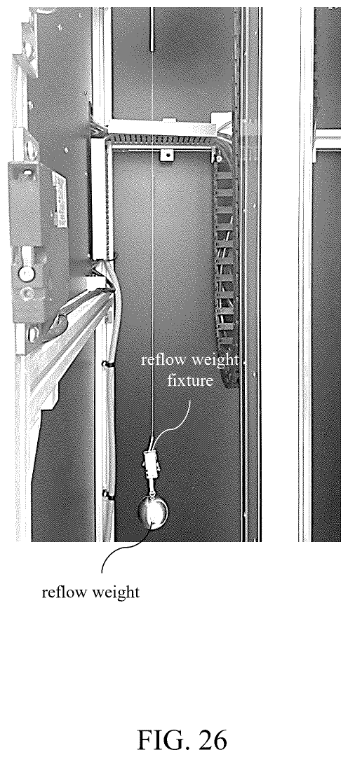

FIG. 26 illustrates the process of attaching reflow weights to the guidewire assembly, in accordance with some embodiments. In some embodiments, the process includes clipping a reflow weight onto the reflow weight fixtures that are secured to each of the distal ends of the guidewires.

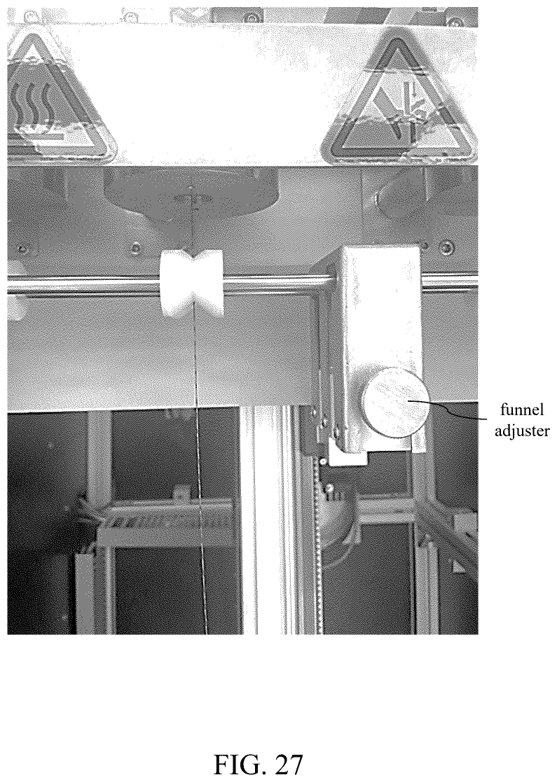

FIG. 27 illustrates the process of closing the funnels around the guidewire assembly within the reflow tower, in accordance with some embodiments. In some embodiments, the process includes manipulating a funnel adjuster (e.g., nob) to activate the mechanism that closes the funnels around the parts. In some embodiments, the funnels are configured to loosely hold the wires in place, providing sufficient clearance to prevent snagging on the heat-shrink material during thermal processing. In some embodiments, the process includes adjusting the spacing of the funnels using the control knobs if necessary to maintain proper alignment and clearance before initiating the reflow cycle.

In some embodiments, the process includes causing the carriage to move to its start position. Once the carriage reaches the start position, the process includes initiating the reflow cycle. In some embodiments, the process includes removing the reflow weights from the guidewire after the reflow cycle is complete, removing the guidewire from the reflow tower, detaching the reflow weight fixtures, and/or allowing the guidewire to reach room temperature. In some embodiments, the process includes gently peeling the FEP heat-shrink material from the guidewire until it is fully removed. In some embodiments, the process includes performing a 100% visual inspection of the polymer segment to verify that there is no wire exposure, no segment gaps, and no incomplete reflow, including any point where polymer coverage does not extend around the entire diameter of the coated segment. If any nonconformance is observed, the part is removed from production, in accordance with some embodiments.

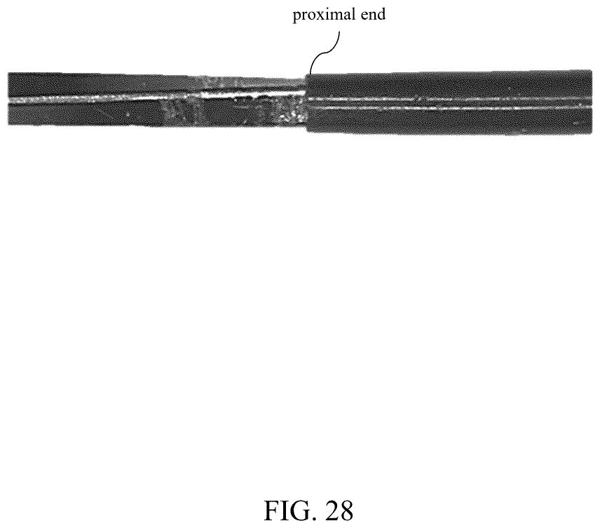

FIG. 28 illustrates the process of cutting the proximal end of the polymer extrusions evenly, in accordance with some embodiments. In some embodiments, the process includes trimming the proximal end of the extrusions to achieve a uniform edge and inspecting the location to confirm that the cut is clean and free of irregularities.

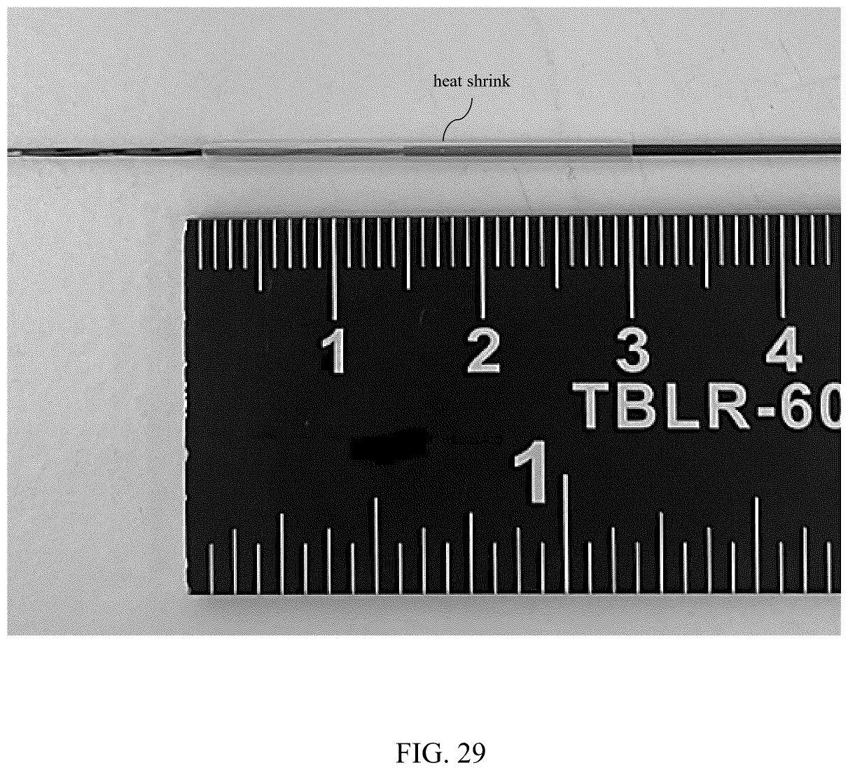

FIG. 29 illustrates the process of preparing a heat-shrink segment for proximal tipping, in accordance with some embodiments. In some embodiments, the process includes cutting a piece of fluorinated ethylene propylene (FEP) heat shrink material to a length of 3 centimeters and forming a slit at one end. In some embodiments, the process includes loading the FEP heat shrink material over the proximal end of the polymer extrusions and centering it to ensure uniform coverage during the subsequent thermal bonding operation.

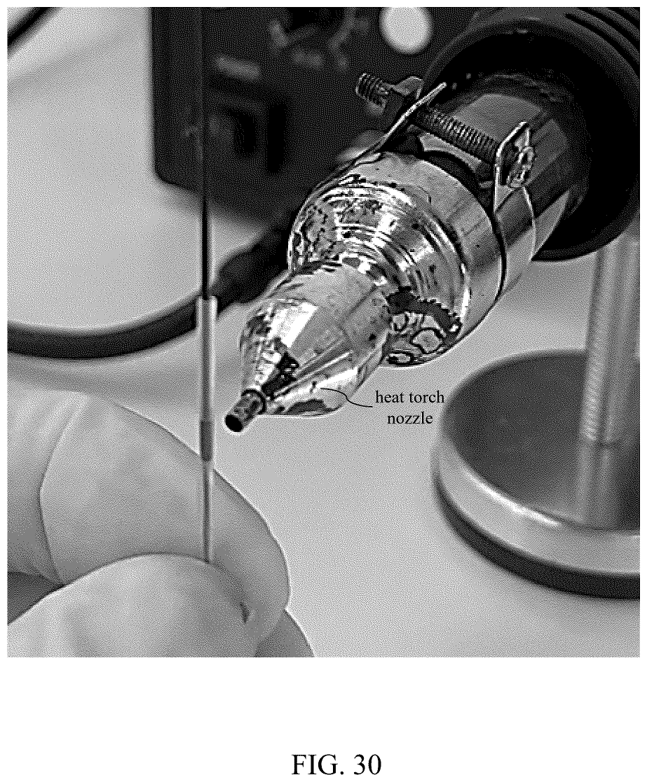

FIG. 30 illustrates the process of applying heat to shrink an FEP segment over the proximal end of the polymer extrusions, in accordance with some embodiments. In some embodiments, the process includes using a calibrated heat torch set to 550 degrees Fahrenheit with a heat torch nozzle to begin shrinking the FEP approximately 0.5 centimeters distal of the proximal end of the extrusions. In some embodiments, the process includes continuing the shrink operation in a proximal direction while rotating the assembly to ensure uniform shrinkage and secure bonding of the FEP around the extrusions. In some embodiments, the process includes gently peeling the fluorinated ethylene propylene (FEP) heat shrink along the slit until it is completely removed from the part. In some embodiments, the process includes performing a one-hundred percent visual inspection of the proximal end of the extrusions to confirm that the cut is clean and that the extrusions are tapered onto the helical region of the wire. If the proximal end is not clean cut or properly tapered, the part is removed from production.

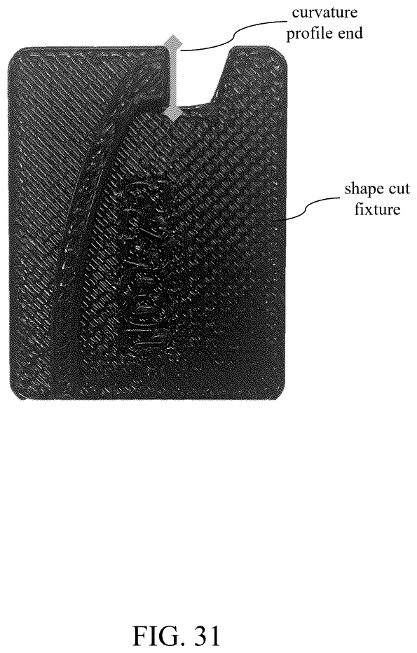

FIG. 31 illustrates the process of trimming the distal tip of the guidewire using a shape cut fixture, in accordance with some embodiments. In some embodiments, the process includes gently placing the distal tip of the wire onto the shape cut fixture so that the curvature of the wire is positioned tightly against the wall of the fixture. In some embodiments, the process includes using flush cutters to cut the distal tip of the wire at the point where the curvature profile ends, ensuring a precise termination of the curved section for proper distal shaping.

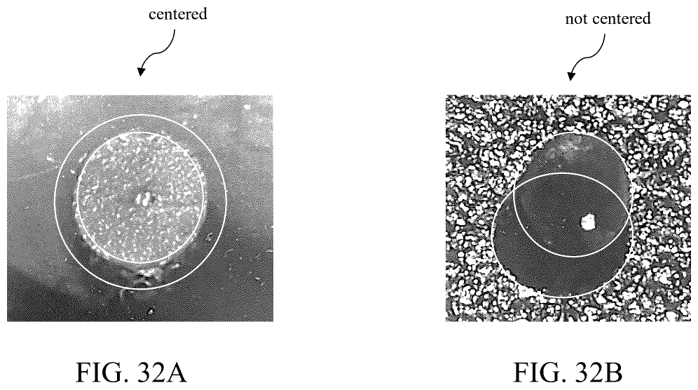

FIG. 32A illustrates a guidewire tip that is properly centered within the surrounding polymer after cutting, in accordance with some embodiments. FIG. 32B illustrates a guidewire tip that is not centered within the polymer after cutting. In some embodiments, the process includes inspecting the distal tip to confirm that the wire is centered within the polymer coating. If the wire is not centered, the part is removed from production to maintain quality and safety standards, in accordance with some embodiments.

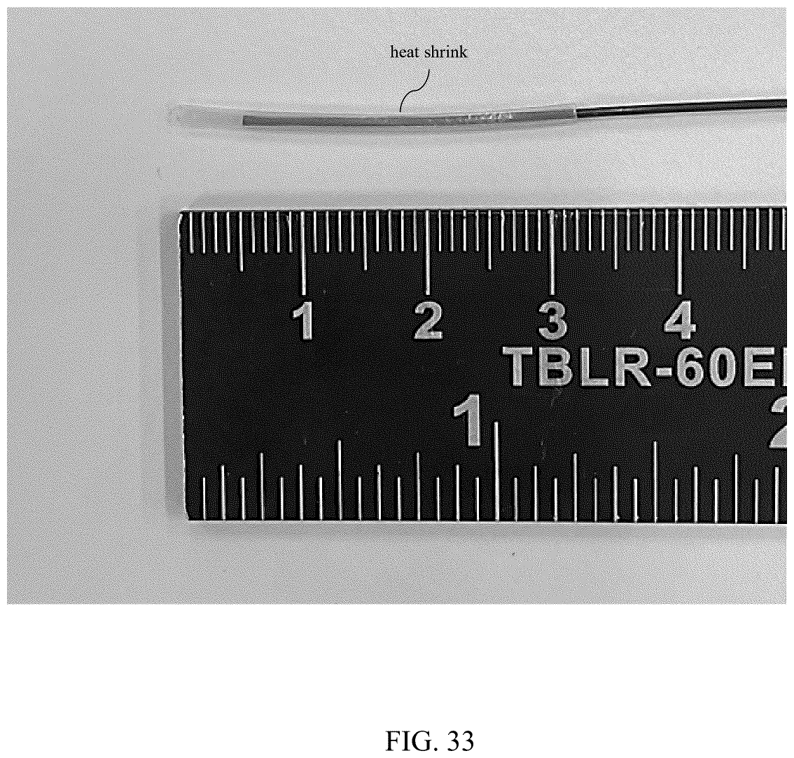

FIG. 33 illustrates the process of preparing and applying a heat-shrink segment to encapsulate the distal tip of the guidewire, in accordance with some embodiments. In some embodiments, the process includes cutting a piece of fluorinated ethylene propylene (FEP) heat shrink material to a length of 3 centimeters and forming a slit at one end. In some embodiments, the process includes loading the FEP heat shrink material over the distal tip of the guidewire until the heat-shrink segment is positioned approximately 0.5 centimeters distal of the tip. In some embodiments, the process includes using a calibrated heat torch set to 380 degrees Fahrenheit with a heat torch nozzle to apply minimal heat to the distal tip, encapsulating the wire tip within the polymer coating.

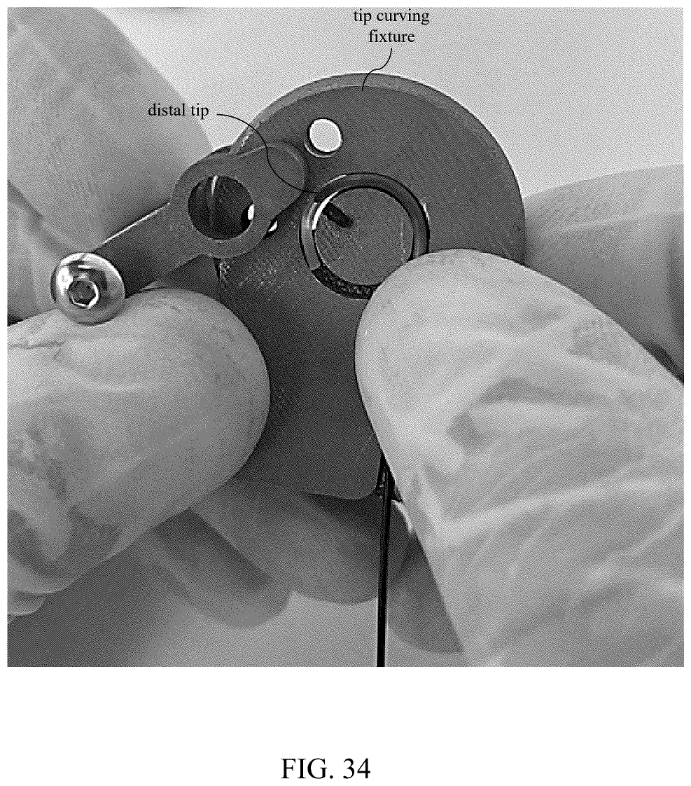

FIG. 34 illustrates the process of positioning the encapsulated distal tip of the guidewire within a tip curving fixture, in accordance with some embodiments. In some embodiments, the process includes placing the distal tip of the part into the curvature of the tip curving fixture so that the guidewire tip aligns with the position indicator, shown as a marked black line. In some embodiments, the process includes closing the front arm of the tip curving fixture and locking the part securely in place by tightening a cap screw.

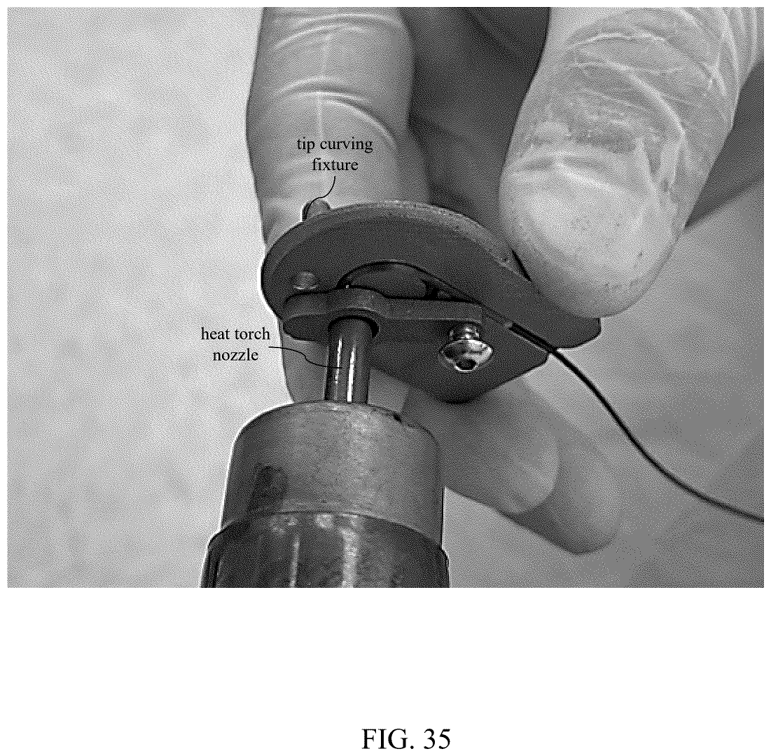

FIG. 35 illustrates the process of applying heat to the distal tip of the guidewire during the tipping operation, in accordance with some embodiments. In some embodiments, the process includes using a calibrated heat torch set to 410 degrees Fahrenheit with a heat torch nozzle to heat the tip of the part for approximately three seconds on each side while the part is secured in the tip curving fixture.

In some embodiments, the process includes gently removing the part from the tip curving fixture and allowing the part to reach room temperature. In some embodiments, the process includes peeling the FEP heat shrink material along the slit starting at the distal tip until it is completely removed from the part. In some embodiments, the process includes performing a one-hundred percent visual inspection of the distal tip to confirm that the tip is rounded and that no wire is exposed. If the tip is not rounded or if exposed wire is present, the part is removed from production. In some embodiments, the process includes verifying the tip shape curvature using a round curve tip shape gauge fixture by laying the shaped tip on the fixture to confirm that the curvature falls within the acceptable window criteria. If the tip shape curvature is out of range, the part is removed from production.

In some embodiments, the process includes visually inspecting the guidewire for nicks, cuts, or kinks along its length, exposed wire in the distal polymer-coated region, and embedded foreign material greater than 0.2 square millimeters using a TAPPI chart. In some embodiments, the process includes verifying that the diameter of the guidewire is less than or equal to 0.035 inches using a calibrated laser micrometer and confirming that the overall length is 180 centimeters±2 centimeters using a calibrated 200-centimeter ruler. Any part that fails these inspection criteria is removed from production.

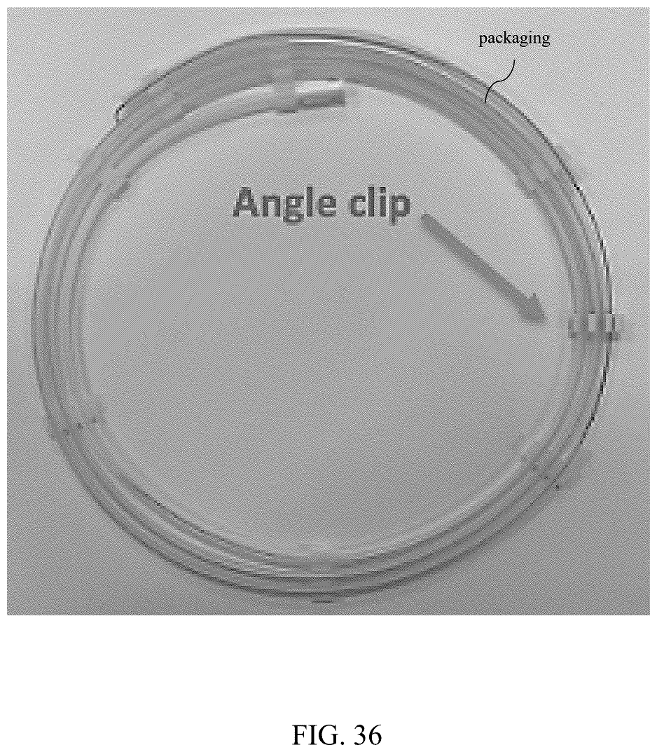

FIG. 36 illustrates the packaging process for the guidewire, in accordance with some embodiments. In some embodiments, the process includes blowing out and wiping down the packaging tube assemblies and all parts using an ionizing air gun to remove particulates. In some embodiments, the process includes loading the guidewire into the packaging tube assembly with the proximal end inserted first until approximately 1 centimeter of the distal tip remains exposed from the tube, and resting the distal portion of the guidewire on an angle clip. In some embodiments, the process includes placing each coiled tube into a recloseable bag (e.g., measuring 10 inches by 12 inches) and printing labels that include part description, revision, lot number, purchase order number, and quantity. In some embodiments, the process includes placing up to ten labeled.

In some embodiments, the present disclosure includes a system comprising a multi-edge profile guidewire in combination with one or more catheters having a textured inner surface. This configuration has been found to significantly reduce friction even further than when used with a catheter that includes a substantially smooth inner surface. As discussed above, the multi-edge profile guidewire includes discrete peaks and valleys along its helical section, which minimizes the contact area between the guidewire and the catheter lumen. When paired with a catheter inner surface that includes a textured pattern, such as ribbing or recessed valleys formed by reinforcement structures, as in the non-limiting examples herein, the system creates alternating zones of contact and clearance. These zones reduce drag forces during advancement and withdrawal, enabling smoother navigation through tortuous anatomy.

In some embodiments, the textured inner surface of the catheter includes peaks formed by reinforcement structures and valleys formed by polymer encasement sleeves. The valleys are configured to reduce the contact area by between approximately fifteen percent and eighty-five percent relative to a smooth inner surface. When the multi-edge profile guidewire traverses this textured lumen, the peaks of the guidewire align intermittently with the peaks of the catheter, while the valleys of the guidewire align with recessed regions of the catheter. This alternating alignment reduces continuous surface engagement, thereby lowering frictional resistance. Empirical testing has demonstrated that this combination decreases track force, resulting in improved torque transmission and enhanced operator control.

In some embodiments, the synergistic effect of the multi-edge profile guidewire and the textured catheter lumen is particularly advantageous in procedures requiring rapid device exchanges or navigation through highly tortuous vascular segments. Conventional systems employing smooth inner surfaces often exhibit stick-slip behavior, where the guidewire alternates between resistance and sudden release during advancement. In contrast, the disclosed system provides a controlled, low-resistance interface that eliminates stick-slip and enables continuous, predictable movement. This improvement reduces procedure time and enhances safety by minimizing the risk of vessel trauma associated with abrupt guidewire motion.

In some embodiments, the combination of structural and surface features described herein allows for the elimination of traditional liners, such as polytetrafluoroethylene (PTFE), which are commonly used to reduce friction in catheter lumens. By leveraging the textured inner surface and hydrophilic coatings in conjunction with the multi-edge profile guidewire, the system achieves superior lubricity without the drawbacks of PTFE liners, such as increased wall thickness and reduced flexibility. This system enables thinner catheter walls, improved kink resistance, and enhanced trackability, while maintaining or exceeding the performance metrics of conventional PTFE-lined catheters.

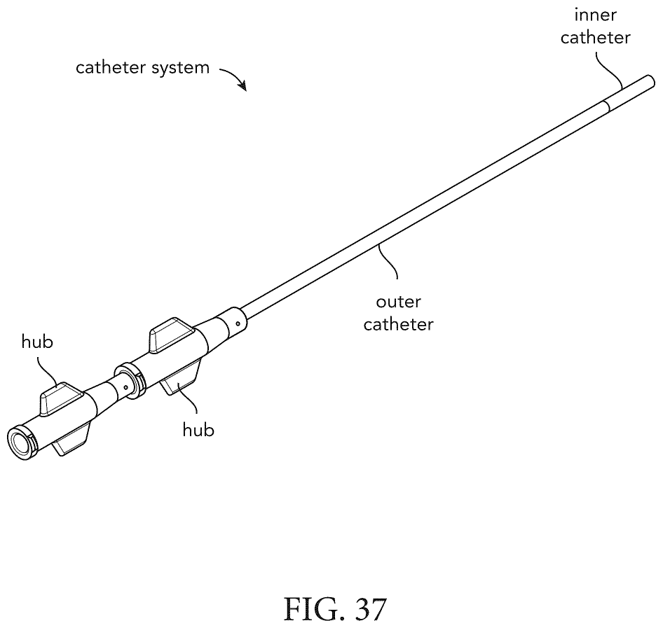

FIG. 37 illustrates a perspective view of a catheter system, according to some embodiments. In some embodiments, the catheter system includes an outer catheter and an inner catheter, as illustrated in FIG. 1. In some embodiments, the outer catheter includes a hub, and the inner catheter includes a hub. The catheter system will be described in greater detail throughout this disclosure.

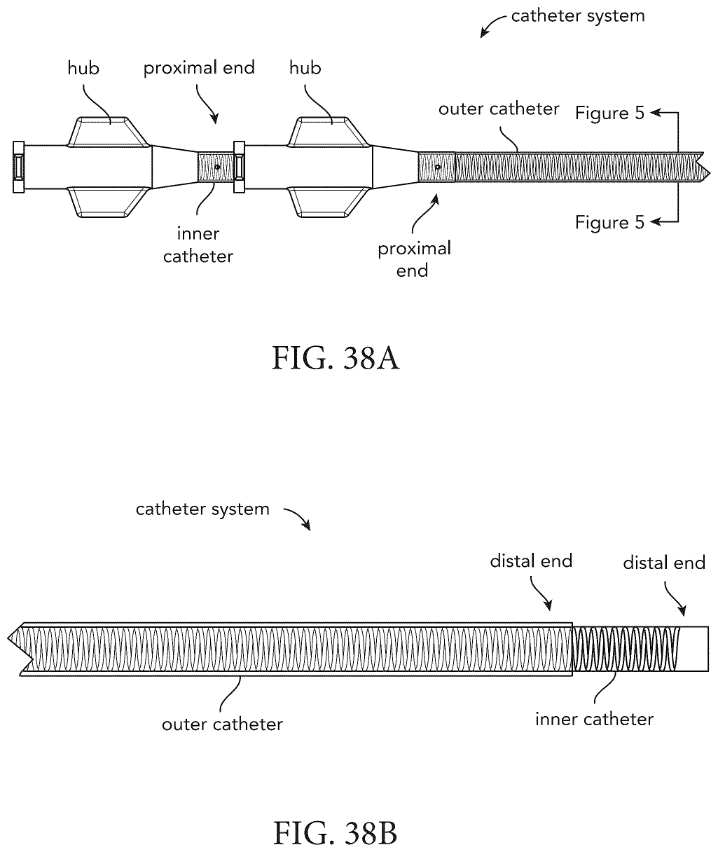

FIG. 38A and FIG. 38B further illustrate the catheter system, according to some embodiments. In some embodiments, the catheter system includes an outer catheter and an inner catheter, as illustrated in FIG. 38A and FIG. 38B. In some embodiments, the outer catheter includes a hub, and the inner catheter includes a hub. As shown in FIG. 38A, the hub may be located at the proximal end of the outer catheter, and the hub may be located at the proximal end of the inner catheter. In some embodiments, the outer catheter includes a distal end located opposite the proximal end, and the inner catheter includes a distal end located opposite the proximal end. In some embodiments, the outer catheter and/or inner catheter includes an outer surface that is smoother than an inner surface.

In some embodiments, the outer catheter may be sized and configured to at least partially receive the inner catheter, as illustrated in FIG. 38A and FIG. 38B. In some embodiments, the outer catheter may also be sized and configured to receive one or more traversing structures, such as the guidewire described herein, a microcatheter, an intermediate catheter, and/or a stent retriever, to name a few non-limiting examples. In some embodiments, the catheter system includes a combination of an inner and outer device (i.e., the inner and outer catheters) that can be used concentrically, with the inner device inside of the outer device. In some embodiments, the outer and/or inner catheters can be used individually. For example, during a procedure, such as a thrombectomy, the outer catheter may be inserted into the patient first in an initial attempt to track the outer catheter distally within the anatomy to a surface of a clot. If the outer catheter successfully tracks the surface of the clot, an aspiration force may be applied to the outer catheter, thereby removing the clot through the outer catheter. In some embodiments, if the outer catheter is unsuccessful in tracking to the surface of the clot, the outer catheter may still serve as a guide or support catheter to help deliver the inner catheter through the outer catheter to the surface of the clot.

In some embodiments, the system is configured to accommodate advanced guidewire designs, such as a multi-edge profile guidewire featuring a helical twist. The inclusion of such a guidewire within the working lumen of the outer catheter can reduce frictional resistance and improve torque transmission during navigation through tortuous anatomy. By minimizing contact area through discrete peaks and valleys formed by the helical structure of the guidewire, the operator can achieve greater precision when advancing the inner catheter or other interventional devices. This interaction between the catheter system and the multi-edge profile guidewire enhances overall trackability and control, particularly in procedures requiring rapid and accurate positioning.

In some embodiments, a method of using only a single device (i.e., the outer catheter) to remove the clot allows for procedures to be more efficient than current procedure practices, which often involve several steps of introducing and removing several devices. In some embodiments, the catheter system, including the inner catheter and outer catheter, allows patient anatomy to drive the procedure, rather than following the same steps for every patient, as is the current practice.

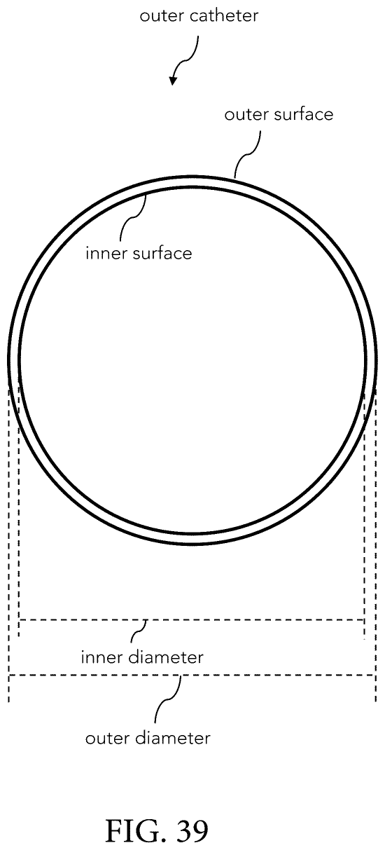

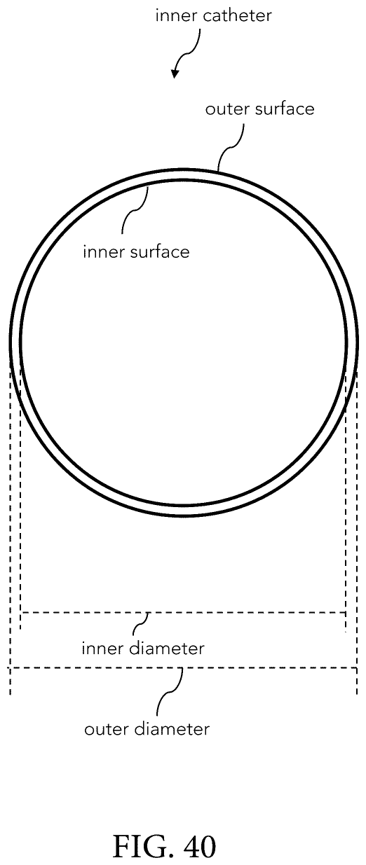

FIGS. 39 and 40 illustrate cross-sectional views of the outer catheter and inner catheter, respectively. In some embodiments, the outer catheter may include an outer surface defining an outer diameter and an inner surface defining an inner diameter. The outer diameter and inner diameter may each define a broad range of dimensions, including, for example, 0.111 inches for the outer diameter and 0.100 inches for the inner diameter, according to some embodiments. As illustrated in FIG. 40, in some embodiments, the inner catheter may also include an outer surface defining an outer diameter and an inner surface defining an inner diameter. In some embodiments, the outer diameter of the outer surface defines a measurement of 0.098 inches, and the inner diameter of the inner surface defines a measurement of 0.088 inches, although any inner and/or outer measurement can be used, as the dimensions listed here, for both the outer catheter and the inner catheter, are included as non-limiting examples. In some embodiments, the outer catheter and inner catheter may both define a wide range of dimensions not explicitly listed in this disclosure. For example, the outer catheter and inner catheter may define outer and/or inner diameter dimensions between 0.003 inches and 0.18 inches, according to some embodiments.

FIG. 39 and FIG. 40 illustrate cross-sectional views of the outer catheter and inner catheter, respectively. In some embodiments, the outer catheter may include an outer surface defining an outer diameter and an inner surface defining an inner diameter. The outer diameter and inner diameter may each define a broad range of dimensions, including, for example, 0.111 inches for the outer diameter and 0.100 inches for the inner diameter, according to some embodiments. As illustrated in FIG. 40, in some embodiments, the inner catheter may also include an outer surface defining an outer diameter and an inner surface defining an inner diameter. In some embodiments, the outer diameter of the outer surface defines a measurement of 0.098 inches, and the inner diameter of the inner surface defines a measurement of 0.088 inches, although any inner and/or outer measurement can be used, as the dimensions listed here, for both the outer catheter and the inner catheter, are included as non-limiting examples. In some embodiments, the outer catheter and inner catheter may both define a wide range of dimensions not explicitly listed in this disclosure. For example, the outer catheter and inner catheter may define outer and/or inner diameter dimensions between 0.003 inches and 0.18 inches, according to some embodiments.

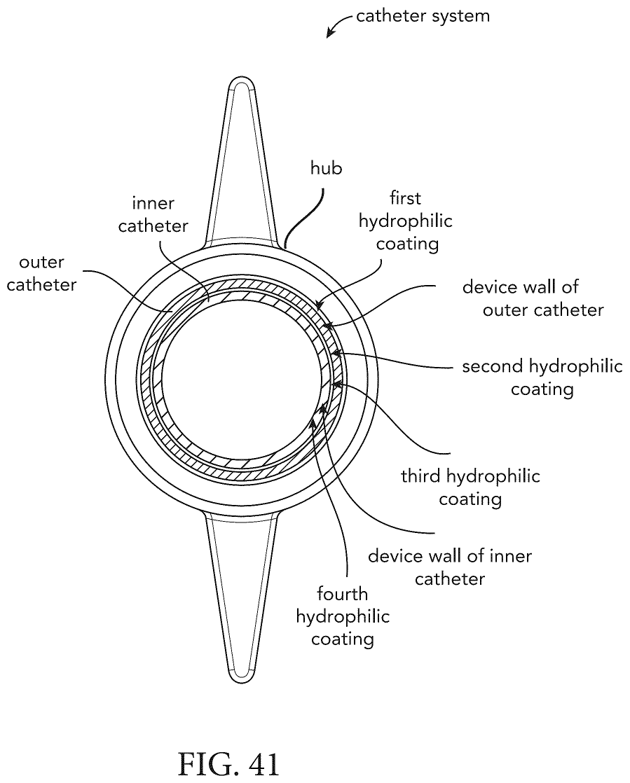

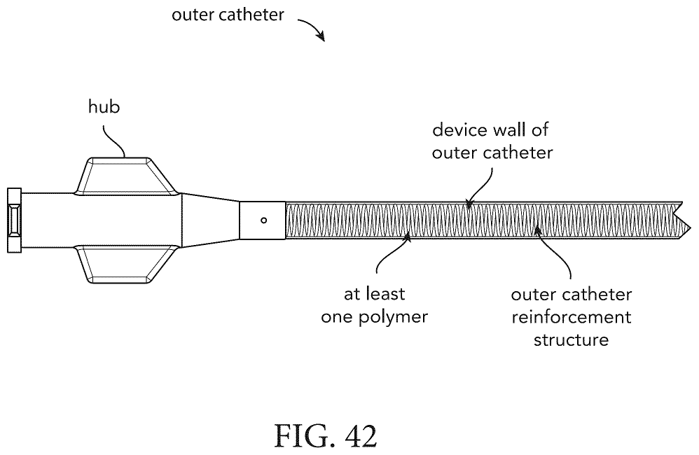

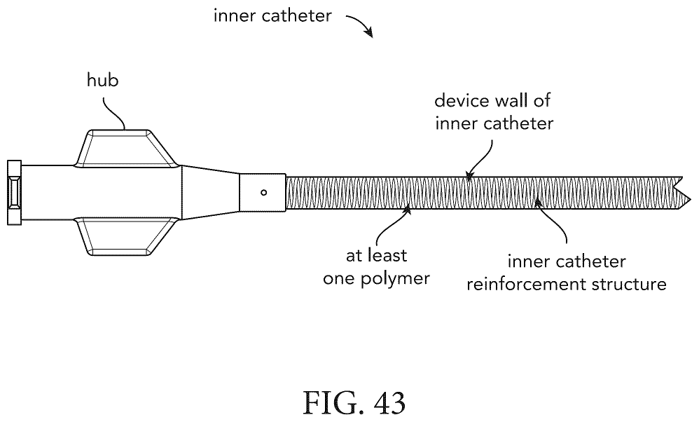

As indicated in FIG. 38A, FIG. 41 illustrates a cross-section of the catheter system, including both the outer catheter and inner catheter, as well as the hub of the outer catheter according to some embodiments. In some embodiments, as shown in FIG. 41, the outer catheter comprises a device wall of the outer catheter, and the inner catheter comprises a device wall of the inner catheter. The device walls will be discussed further with reference to FIG. 42 and FIG. 43. In some embodiments, the catheter system may also include a first hydrophilic coating, a second hydrophilic coating, a third hydrophilic coating, and a fourth hydrophilic coating, as shown in FIG. 41.

In some embodiments, the first hydrophilic coating is located on the outer surface of the outer catheter, and the second hydrophilic coating is located on the inner surface of the outer catheter. In some embodiments, the device wall of the outer catheter is located between the first hydrophilic coating and the second hydrophilic coating. In some embodiments, the third hydrophilic coating is located on the outer surface of the inner catheter, and/or the fourth hydrophilic coating is located on the inner surface of the inner catheter. In some embodiments, the device wall of the inner catheter is located between the third hydrophilic coating and the fourth hydrophilic coating. In some embodiments, each of the first, second, third, and fourth hydrophilic coatings may extend along a surface extending between the proximal ends and distal ends of the outer and inner catheters. In some embodiments, the surface extends substantially an entire length of the catheters. In some embodiments, the surface may extend less than a full length, such as 50%, 25%, or 10% of the entire length. In some embodiments, each of the hydrophilic coatings is configured to cover a distalmost portion, such as 15 centimeters, of the outer and inner catheters. It should be noted that, in some embodiments each of the hydrophilic coatings is configured to cover any size portion of the catheters. It should also be noted that each of the hydrophilic coatings does not necessarily define the same length, though they may each define the same length according to some embodiments.

In some embodiments, each of the first hydrophilic coating, second hydrophilic coating, third hydrophilic coating, and fourth hydrophilic coating may comprise the same material and thickness. In some embodiments, the thickness of each hydrophilic coating is between 0.0001 and 0.001 inches. The term “hydrophilic coating” is a species of lubricious coatings that reduce friction and increase trackability of the outer and inner catheters as they move within vessels and/or within one another (e.g., the inner catheter moving within the outer catheter) according to some embodiments. Some non-limiting examples of lubricious coatings according to some embodiments include hydrophilic coatings, silicone coatings, PTFE dust, and any other suitable lubricants. In some embodiments, coating the device wall with one or more lubricious coatings allows the device walls to be thinner than traditional device walls while also improving the performance of the catheters. In some embodiments, the device walls may include a thickness between 0.001 and 0.04 inches.

Referring now to FIG. 42, the outer catheter is shown, in accordance with some embodiments. As previously discussed, the outer catheter includes a device wall according to some embodiments. In some embodiments, as illustrated in FIG. 42, the device wall comprises at least one polymer and an outer catheter reinforcement structure. In some embodiments, the at least one polymer may also be referred to as a polymer jacket structure. In some embodiments, the at least one polymer is configured to provide at least one of flexibility and structural support to the outer catheter and/or the inner catheter. In some embodiments, the outer catheter reinforcement structure may comprise a metallic braid and/or coil structure, with the at least one polymer filling any space within the braid and/or coil structure. In some embodiments, the at least one polymer may also cover the outer catheter reinforcement structure. In some embodiments, the outer catheter reinforcement structure is configured to provide stiffness to a proximal portion of the outer catheter and flexibility to a distal portion of the outer catheter. In some embodiments, the amount, coil tightness, and/or composition of the outer catheter reinforcement structure and/or inner catheter may vary depending on the location along the length of the outer catheter and/or length of inner catheter.

Similar to FIG. 42, FIG. 43 shows the inner catheter including the device wall comprising the at least one polymer and the inner catheter reinforcement structure, according to some embodiments. In some embodiments, the inner catheter reinforcement structure is substantially similar in construction to the outer catheter reinforcement structure, including the structure immersed in the at least one polymer. In some embodiments, the inner catheter reinforcement structure may comprise one or both of a coil and braided structure. In some embodiments, the inner catheter reinforcement structure is configured to provide stiffness to a proximal portion of the inner catheter and flexibility to a distal portion of the inner catheter. In some embodiments, the amount, coil tightness, and/or composition of the inner catheter reinforcement structure may vary depending on the location along the length of the inner catheter. In some embodiments, the device wall of the outer catheter and the device wall of the inner catheter are substantially the same construction and may comprise the same type of polymer(s) in the at least one polymer, as well as the same type of braid and/or coil structure in the outer catheter reinforcement structure and/or inner catheter reinforcement structure. In some embodiments, the inner catheter may be considered a scaled-down version of the outer catheter, with the same elements but smaller dimensions.

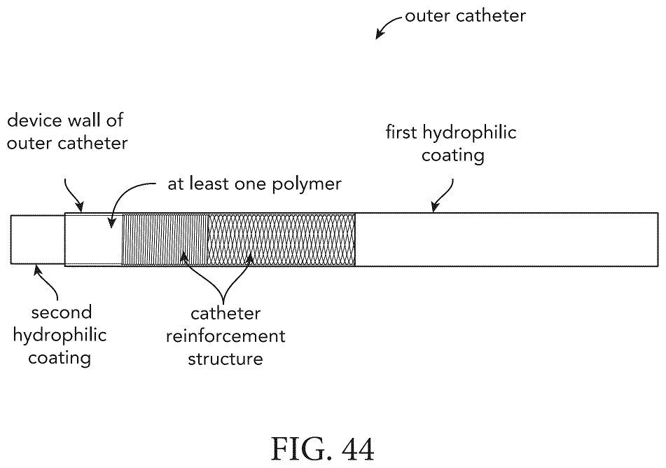

FIG. 44 is also similar to FIG. 42 in that it illustrates another view of the outer catheter, including the various layers of materials according to some embodiments. Included in FIG. 44 are the first hydrophilic coating, the second hydrophilic coating, the device wall, the at least one polymer, and the outer catheter reinforcement structure. In some embodiments, the device wall may have a structure where the at least one polymer and the outer catheter reinforcement structure are melded together, with the first hydrophilic coating located on the outer surface and the second hydrophilic coating located on the inner surface.

In some embodiments, the device wall has a “sandwich” structure comprising two layers of the at least one polymer directly coupled together, with the outer catheter reinforcement structure between the polymer layers. As previously discussed, the outer catheter reinforcement structure may comprise a braid and/or coil structure. In some embodiments, the outer catheter reinforcement structure and/or inner catheter reinforcement structure may each include individual coil and/or braid structures, as indicated by the different appearances of the outer catheter reinforcement structure in FIG. 44. For example, the coil structure is represented by the portion of the outer catheter reinforcement structure to the left in FIG. 44, while the braid structure is represented by the portion of the outer catheter reinforcement structure to the right in FIG. 44 according to some embodiments. In some embodiments, the “sandwich” style device wall comprises an inner layer of at least one polymer, the coil structure on top of the inner polymer layer, the braid structure on top of the coil structure, and an outer layer of at least one polymer. In some embodiments, in the “sandwich” style, the device wall also includes the first hydrophilic coating and the second hydrophilic coating.

In some embodiments, the “sandwich” style device wall allows for a larger (more open) coil pitch in the coil structure, thereby enabling the outer catheter to be softer than some embodiments where the coil structure has a tighter or more closed pitch. In some embodiments, a softer and more flexible outer catheter can be desirable for certain uses, such as when navigating tortuous anatomy, to give the user (i.e., a medical practitioner) more freedom to move the device at different angles. In some embodiments, this “sandwich” style provides benefits from a manufacturing standpoint, as a more open coil pitch is easier to produce and may include a larger margin of error than a closed pitch. Some embodiments described herein are directed to a method of manufacture of the inner and/or outer catheters.

However, there are benefits to a device wall comprising a tighter pitch coil structure. For example, in some embodiments, the outer catheter reinforcement structure includes a coil defining a pitch smaller than 0.03 inches, and/or the second hydrophilic coating is provided with a substantially solid and/or ribbed surface to adhere to. In this sense, the second hydrophilic coating (as well as the fourth hydrophilic coating of the inner catheter) may be thought of as having a textured, or “ribbed,” surface according to some embodiments. In comparison, in some embodiments, the first hydrophilic coating (and the third hydrophilic coating) may be thought of as having a substantially smooth surface. In some embodiments, the combination of textured and smooth surfaces of the hydrophilic coatings provides just enough friction to allow a user to easily control movement of the outer catheter and the inner catheter. For example, when the second hydrophilic coating has a textured surface and the third hydrophilic coating has a smooth surface, there may be enough friction between the two surfaces to prevent excessive and difficult-to-control sliding of the inner catheter within the outer catheter, as may be the case if both hydrophilic coatings were smooth.

In some embodiments, to ensure a sufficiently solid inner surface of the outer catheter, the coil comprises a 0.002 inch round coil with a 0.004 inch pitch. In some embodiments, a tighter pitch coil may be better for facilitating lubricity of the inner surface of the outer catheter. In some embodiments, a coil with a pitch less than 0.025 inches is desirable. In some embodiments, a sufficiently tight-pitch coil in the outer catheter reinforcement structure, combined with the second hydrophilic coating on the inner surface of the outer catheter, provides enough lubricity to replace the need for a liner, such as a PTFE liner, which is traditionally used in catheter construction. In some embodiments, the coil may comprise a round coil, a flat coil, and/or other types of coil design.

Regardless of the “style” of device wall used (e.g., “sandwich” or tight-pitch coil), the use of a first and second hydrophilic coating on the outer catheter may allow for a thinner, more flexible device wall, as compared to other types of catheter walls without inner and outer coatings according to some embodiments. It should be noted that though FIG. 44 specifically labels the catheter as the outer catheter, the layers shown in FIG. 44 and the preceding discussion also apply to the inner catheter in some embodiments, such that FIG. 44 may be considered as depicting either the outer catheter or the inner catheter.

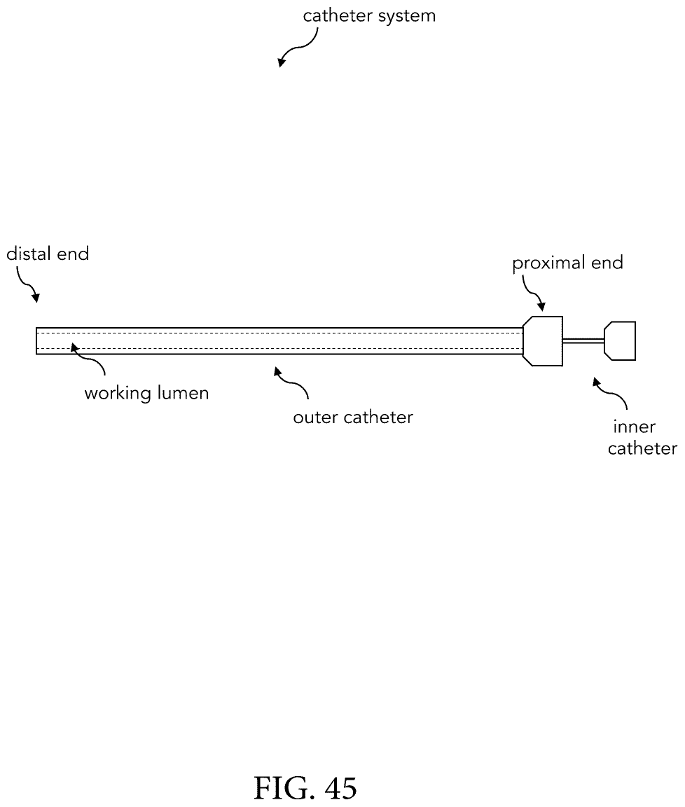

FIG. 45 illustrates a catheter system comprising an outer catheter and an inner catheter, which may be substantially similar in construction to the outer catheter and/or inner catheter described previously according to some embodiments. As shown, the outer catheter may include a proximal end and a distal end located opposite the proximal end. In some embodiments, the outer catheter includes a working lumen extending between the proximal end and the distal end. In some embodiments, the working lumen includes the space inside the catheter configured to allow the passage of various instruments, such as guidewires, microcatheters, stent retrievers, and/or is used for the aspiration of clots and other obstructions within a patient's vasculature. In some embodiments, the working lumen is configured to at least partially receive the inner catheter.

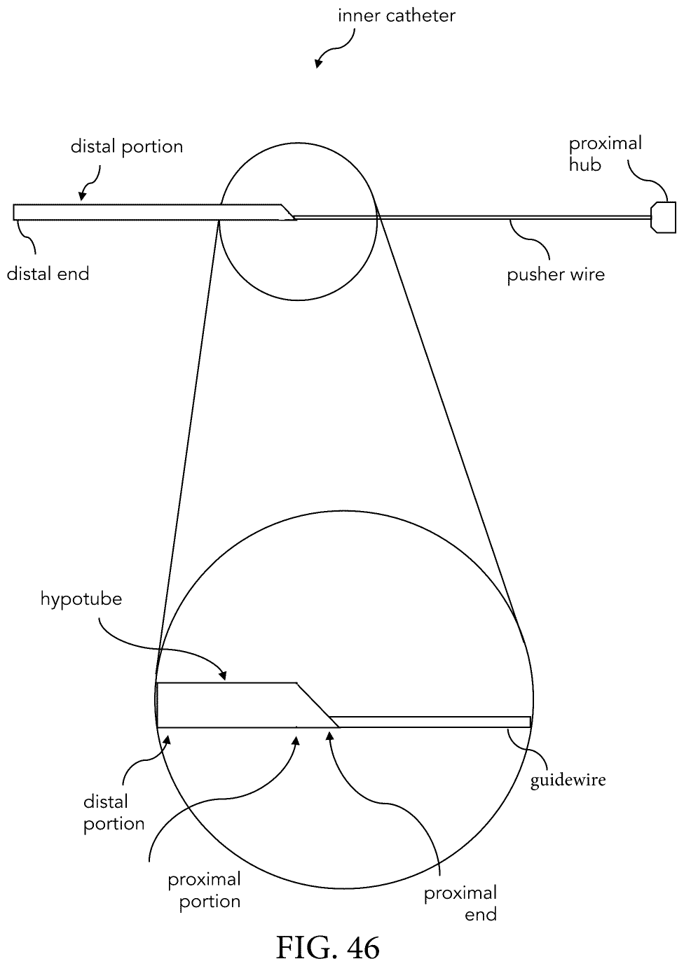

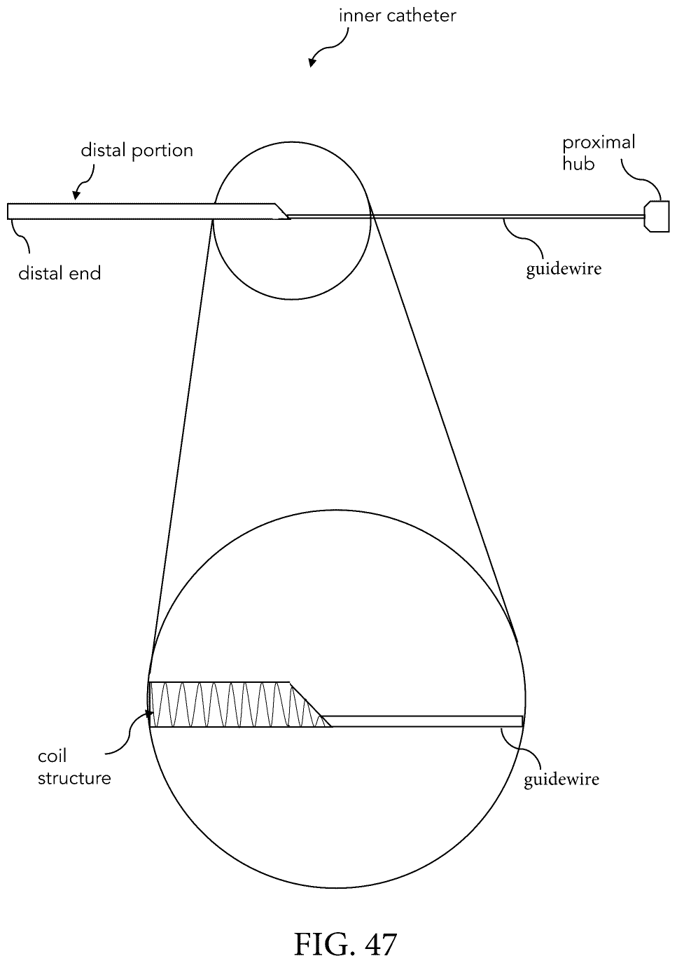

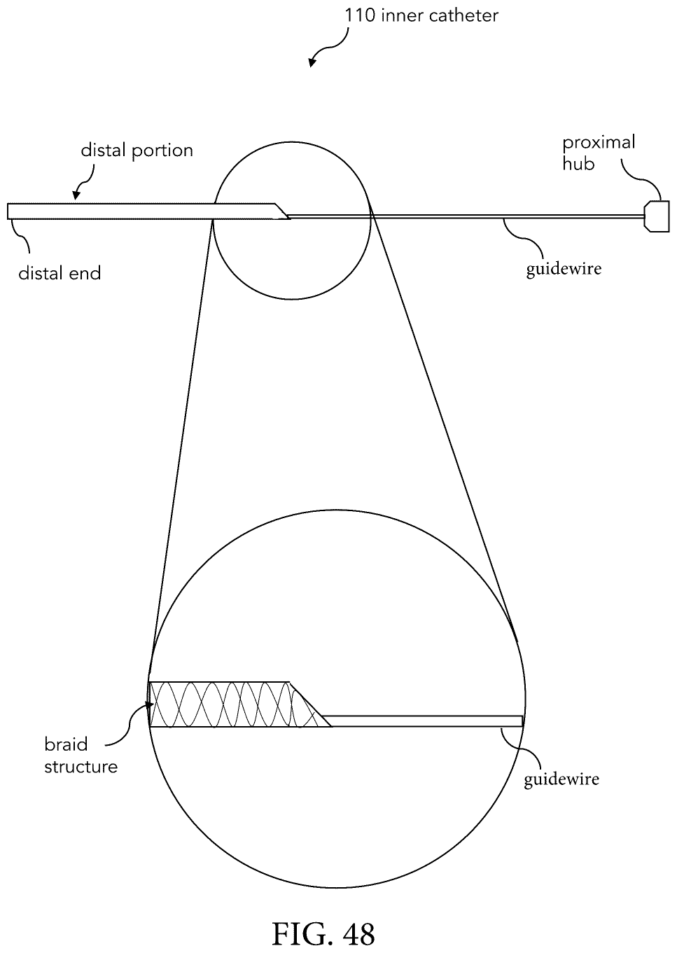

FIG. 46-48 illustrate the inner catheter, which, in some embodiments, comprises a proximal hub, a distal portion having a distal end located opposite the proximal hub, and a guidewire extending between the proximal hub and the distal portion. In some embodiments, the working lumen is configured to at least partially receive the guidewire, as shown in FIG. 45. As illustrated in the inset view of FIG. 46, in some embodiments, the distal portion of the catheter may comprise a wall support structure. While some non-limiting embodiments are directed to a hypotube, any reference to a hypotube may be replaced with any other disclosed support structure when defining the metes and bounds of the system. In some embodiments, the hypotube comprises a stainless steel hypotube. In some embodiments, the hypotube may comprise a nitinol hypotube. In some embodiments, the hypotube is comprised of any suitable material, and, in some embodiments, is a laser-cut hypotube. In some embodiments, the hypotube may comprise a non-laser-cut hypotube. In some embodiments, the hypotube comprises a distal portion and a proximal portion located opposite the distal portion. As shown, the proximal portion is configured to taper to a proximal end coupled to the guidewire. Rather than a hypotube, the distal portion of the inner catheter may comprise a coil structure, as illustrated in FIG. 47, and/or a braid structure, as illustrated in FIG. 48 according to some embodiments. In some embodiments, the distal portion of the inner catheter comprises a combination of the coil structure and the braid structure. In some embodiments, the distal portion may comprise any suitable material configuration and is not limited to the examples shown in the figures and discussed in this disclosure.

As shown in FIG. 46-48, the distal portion of the inner catheter includes a length substantially less than the full length of the inner catheter. In comparison, the outer catheter shown in FIG. 45 may comprise a single tube defining substantially the full length of the outer catheter, minus the proximal end according to some embodiments. In some embodiments, in the catheter system, the outer catheter may be considered a “full catheter” and the inner catheter may be considered a “partial catheter.” In some embodiments, the distal portion of the inner catheter, whether a hypotube, coil structure, and/or braid structure, may include a length of about twenty centimeters. In some embodiments, the distal portion defines a length less than twenty centimeters. In some embodiments, the distal portion may define a length greater than twenty centimeters.

In some embodiments, the guidewire is configured to be fixedly coupled to the distal portion of the inner catheter. In some embodiments, the guidewire is configured to facilitate navigation of the inner catheter through the working lumen of the outer catheter. For example, according to a method of use, during a procedure, a physician (or another qualified medical professional) is configured to “push” the inner catheter through the outer catheter using the proximal hub and/or the guidewire according to some embodiments.

In some embodiments, the guidewire includes a multi-edge profile, such as a square or rectangular cross-section, twisted into a helical configuration with a pitch ranging from approximately 0.0100 to 0.7500 inches, reducing frictional contact with the inner surface of the catheter and surrounding anatomical structures, improving torque transmission and trackability. In some embodiments, the guidewire includes radiused edges between 0.0010 and 0.0200 inches to minimize stress concentrations and enhance smooth navigation. In some embodiments, the guidewire may include variable pitch sections to impart different flexibility characteristics along its length, enabling precise control during advancement through tortuous anatomy. In some embodiments, the guidewire comprises materials such as stainless steel or nitinol to provide strength, elasticity, and fatigue resistance. In some embodiments, the guidewire includes a polymer jacket and hydrophilic coating along at least a portion of its length to further reduce friction and improve lubricity. In some embodiments, the guidewire includes a distal tip that is shapeable or pre-shaped for atraumatic entry into target vessels, and may include radiopaque elements for visualization under imaging modalities. Other features of the guidewire are discussed in relation to FIGS. 1-36.

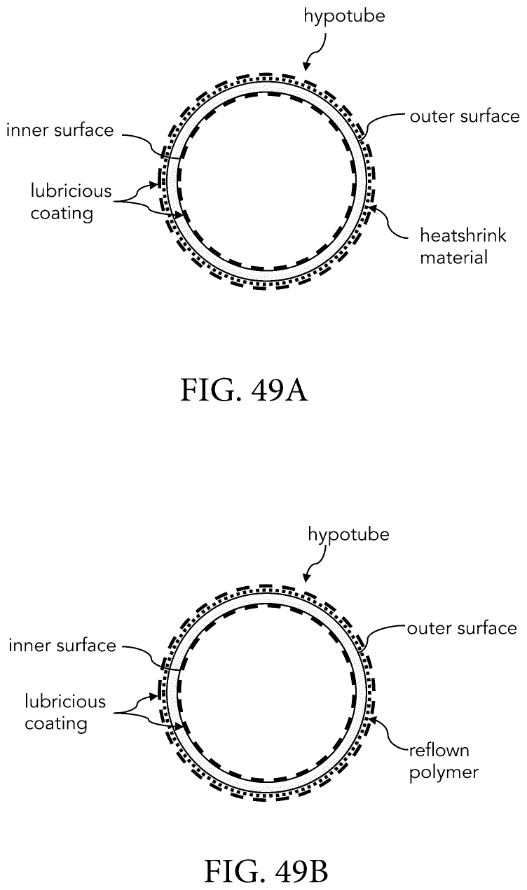

FIGS. 49A and 49B illustrate cross-sectional views of the hypotube according to some embodiments. In some embodiments, the hypotube comprises an inner surface and an outer surface located opposite the inner surface. In some embodiments, the outer surface is covered in a heat shrink material, as shown in FIG. 49A. In some embodiments, the heat shrink material comprises a material laminated, fused, and/or melted onto the outer surface of the hypotube. In some embodiments, at least a portion of the heatshrink material and at least a portion of the inner surface of the hypotube are coated with a lubricious coating. In some embodiments, substantially the entirety of the heat shrink material and substantially the entirety of the inner surface are coated with the lubricious coating. In some embodiments, at least a portion of the heat shrink material and substantially the entirety of the inner surface is coated with the lubricious coating. In some embodiments, substantially the entirety of the heat shrink material and at least a portion of the inner surface is coated with the lubricious coating. In some embodiments, the inner surface varies in diameter along the length of the catheter.

In some embodiments, the lubricious coating may comprise a hydrophilic coating. In some embodiments, the lubricious coating comprises silicone. In some embodiments, the lubricious coating may comprise any suitable type of coating, and is not intended to be limited to the examples discussed in this disclosure. In some embodiments, the lubricious coating helps facilitate smooth navigation of the hypotube through the working lumen of the outer catheter. In some embodiments, where the inner catheter extends distally from the outer catheter, the lubricious coating may also help facilitate smooth navigation of the hypotube through a patient's vasculature. In some embodiments, the lubricious coating on the inner surface of the hypotube facilitates smooth movement of a secondary device (e.g., a guidewire, microcatheter, specialized device, etc.) through the hypotube.

FIG. 49B illustrates that, in some embodiments, the outer surface of the hypotube is covered in a reflown polymer rather than a heat shrink material according to some embodiments. In some embodiments, at least a portion of the reflown polymer and at least a portion of the inner surface of the hypotube is coated with the lubricious coating. In some embodiments, substantially the entirety of the reflown polymer and substantially the entirety of the inner surface of the hypotube are coated with the lubricious coating. In some embodiments, at least a portion of the reflown polymer and substantially the entirety of the inner surface is coated with the lubricious coating. In some embodiments, substantially the entirety of the reflown polymer and at least a portion of the inner surface is coated with the lubricious coating.

In some embodiments, the outer surface of the hypotube is covered with a combination of the heat shrink material and the reflown polymer. In some embodiments, at least a portion of the hypotube includes a PTFE liner rather than the lubricious coating. For example, in some embodiments, half of the hypotube may include a PTFE liner while the other half includes the lubricious coating. In some embodiments, half of the hypotube may include a PTFE liner while the other half includes no lubricious coating. In some embodiments, the hypotube may also include neither a PTFE liner nor a lubricious coating. In some embodiments, the catheter system including the coil structure and/or braid structure, as illustrated in FIGS. 47 and 48, respectively, may also include a heat shrink material, reflown polymer, or combination thereof to cover the coil structure and/or braid structure, as applicable. In some embodiments, the coil structure and/or braid structure may include the lubricious coating as illustrated in FIGS. 49A and 49B.

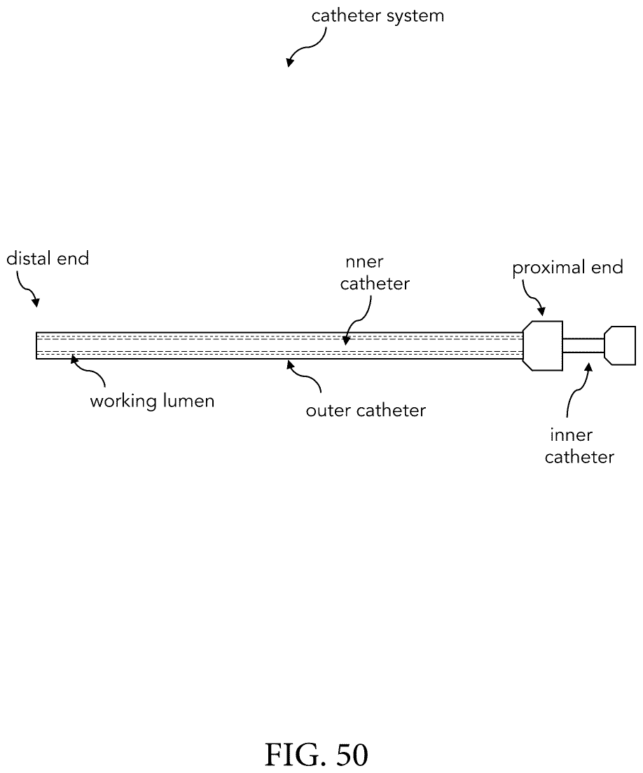

FIG. 50 illustrates a catheter system according to some embodiments. In some embodiments, the catheter system comprises an outer catheter having a proximal end, a distal end located opposite the proximal end, and a working lumen extending between the proximal end and the distal end. In some embodiments, the catheter system may also include an inner catheter, and the working lumen is configured to at least partially receive the inner catheter, as demonstrated in FIG. 50. As with any catheter described herein according to some embodiments, the outer catheter and inner catheter may be made by similar manufacturing methods and/or comprise similar structures, such as those described in relation to FIG. 42-44 and FIG. 59.

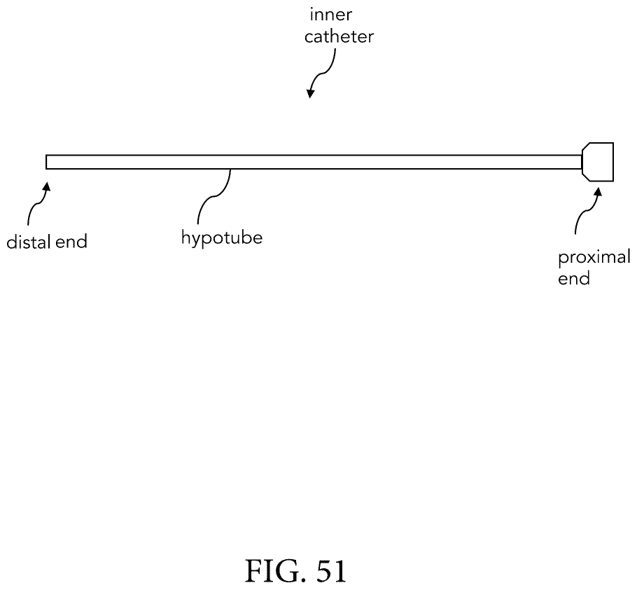

FIG. 51 illustrates the inner catheter in more detail, including the proximal end and the distal end located opposite the proximal end. Unlike the inner catheter of the previous catheter system (shown in FIG. 45-49) according to some embodiments, the inner catheter may comprise a “full” catheter rather than a “partial” catheter. Stated differently, in some embodiments, the inner catheter may comprise a hypotube configured to extend the full length from the proximal end to the distal end. Similar to the hypotube of the inner catheter described previously, in some embodiments, the hypotube of the inner catheter may comprise a laser-cut hypotube. In some embodiments, the inner catheter may comprise a non-laser-cut hypotube. In some embodiments, the hypotube comprises a stainless steel hypotube. In some embodiments, the hypotube may comprise a nitinol hypotube, or a hypotube constructed of any other suitable material.

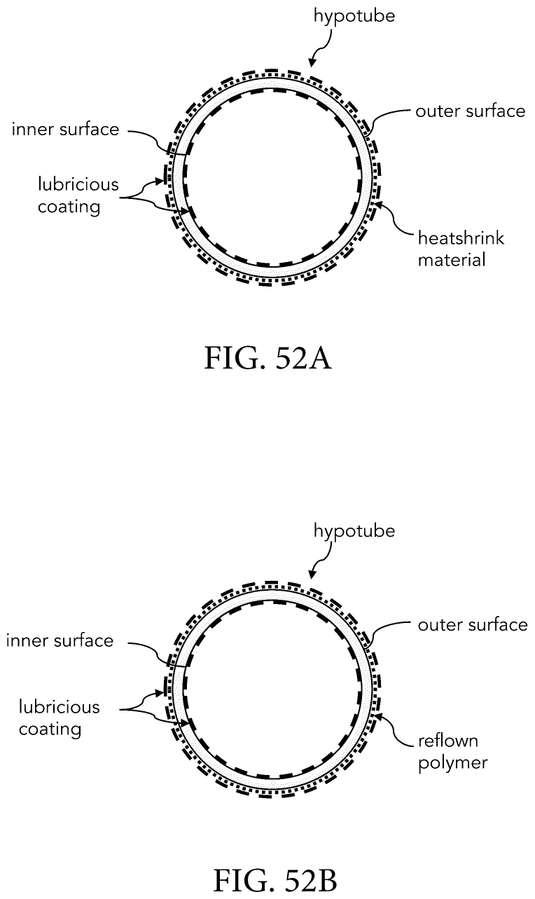

FIGS. 52A and 52B are similar to FIGS. 49A and 49B, though they illustrate the hypotube of the inner catheter described in FIG. 51 according to some embodiments. FIGS. 52A and 52B show cross-sectional views of the hypotube, wherein, in some embodiments, the hypotube comprises an inner surface and an outer surface located opposite the inner surface. In some embodiments, the outer surface is covered in a heat shrink material, as shown in FIG. 52A. In some embodiments, the heat shrink material may comprise a material laminated or melted onto the outer surface of the hypotube. In some embodiments, at least a portion of the heatshrink material and at least a portion of the inner surface of the hypotube are coated with a lubricious coating. In some embodiments, substantially the entirety of the heatshrink material and substantially the entirety of the inner surface are coated with the lubricious coating. In some embodiments, at least a portion of the heat shrink material and substantially the entirety of the inner surface is coated with the lubricious coating. In some embodiments, substantially the entirety of the heat shrink material and at least a portion of the inner surface is coated with the lubricious coating.

In some embodiments, the lubricious coating is substantially similar to or the same as the lubricious coating described in relation to FIGS. 49A and 49B. In some embodiments, the lubricious coating may comprise a hydrophilic coating. In some embodiments, the lubricious coating comprises silicone. In some embodiments, the lubricious coating may comprise any suitable type of coating, and is not intended to be limited to the examples discussed in this disclosure. In some embodiments, the lubricious coating helps facilitate smooth navigation of the hypotube through the working lumen of the outer catheter. In some embodiments, where the inner catheter extends distally from the outer catheter, the lubricious coating may also help facilitate smooth navigation of the hypotube through a patient's vasculature. In some embodiments, the lubricious coating on the inner surface of the hypotube facilitates smooth movement of a secondary device, such as a guidewire. In some embodiments, the guidewire may include a multi-edge profile, such as a square or rectangular cross-section, twisted into a helical configuration with a pitch ranging from approximately 0.0100 to 0.7500 inches. This configuration is designed to reduce frictional contact with the inner surface of the catheter and surrounding anatomical structures, improving torque transmission and trackability. In some embodiments, the guidewire includes radiused edges between 0.0010 and 0.0200 inches to minimize stress concentrations and enhance smooth navigation. In some embodiments, the guidewire may include variable pitch sections to impart different flexibility characteristics along its length, enabling precise control during advancement through tortuous anatomy. In some embodiments, the guidewire comprises materials such as stainless steel or nitinol to provide strength, elasticity, and fatigue resistance. In some embodiments, the guidewire includes a polymer jacket and hydrophilic coating along at least a portion of its length to further reduce friction and improve lubricity. In some embodiments, the guidewire includes a distal tip that is shapeable or pre-shaped for atraumatic entry into target vessels, and may include radiopaque elements for visualization under imaging modalities.

FIG. 52B illustrates that, in some embodiments, the outer surface of the hypotube is covered in a reflown polymer rather than a heat shrink material. In some embodiments, at least a portion of the reflown polymer and at least a portion of the inner surface of the hypotube is coated with the lubricious coating. In some embodiments, substantially the entirety of the reflown polymer and substantially the entirety of the inner surface of the hypotube are coated with the lubricious coating. In some embodiments, at least a portion of the reflown polymer and substantially the entirety of the inner surface is coated with the lubricious coating. In some embodiments, substantially the entirety of the reflown polymer and at least a portion of the inner surface is coated with the lubricious coating.

In some embodiments, the outer surface of the hypotube is covered with a combination of the heat shrink material and the reflown polymer. In some embodiments, at least a portion of the hypotube includes a PTFE liner rather than the lubricious coating. For example, in some embodiments, half of the hypotube may include a PTFE liner while the other half includes the lubricious coating. In some embodiments, half of the hypotube may include a PTFE liner while the other half includes no lubricious coating. In some embodiments, the hypotube may also include neither a PTFE liner nor a lubricious coating. In some embodiments, the inner catheter may comprise, rather than the hypotube, a coil structure and/or braid structure, similar to those illustrated in FIG. 42-44 and FIG. 47-48. In some embodiments, the inner catheter comprising a coil and/or braid structure also includes a heat shrink material, reflown polymer, or combination thereof to cover the coil structure and/or braid structure, as applicable. In addition, embodiments with the coil structure and/or braid structure may include the lubricious coating as illustrated in FIGS. 52A and 52B.

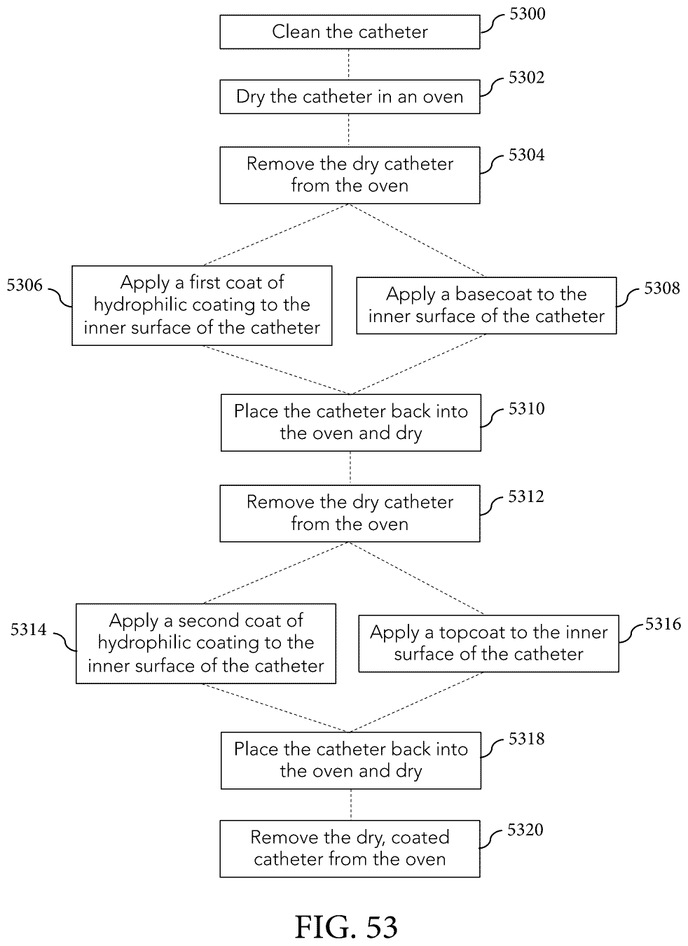

FIG. 53 shows a flowchart illustrating a non-limiting example process of coating and curing an inner surface of a catheter according to some embodiments. For the purposes of this disclosure, the “catheter” recited in FIG. 53 may comprise elements of the catheter system described in FIG. 37-44 (i.e., the outer catheter or the inner catheter), elements of the catheter system described in FIG. 45-49 (i.e., the outer catheter or the inner catheter), and/or elements of the catheter system described in FIG. 50-52. The steps of the process, which include a method of manufacture, should be considered as applying to any of the catheters recited in this disclosure.

In some embodiments, the process shown in FIG. 53 starts with cleaning the catheter, at step 5300. In some embodiments, cleaning the catheter includes flushing the catheter with purified water, isopropyl alcohol (“IPA”), a mix of IPA and water, and/or some other suitable cleansing fluid. In some embodiments, the next step is to dry the catheter in an oven, at step 5302. In some embodiments, the drying step may include placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter according to some embodiments. In some embodiments, the process continues with step 5304: remove the dry catheter from the oven.

Next, in some embodiments, the process can continue in one of two possible steps. One option is to apply a first coat of hydrophilic coating to the inner surface of the catheter, shown at step 5306 according to some embodiments. In some embodiments, a basecoat is applied to the inner surface of the catheter, at step 5308. Both steps 5306 and 5308 may use positive or negative pressure to fill the catheter with either the hydrophilic coating (step 5306) or the basecoat (step 5308) according to some embodiments. In some embodiments, the catheter is filled with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time.

After either step 5306 or step 5308, in some embodiments the process may continue to place the catheter back into the oven to dry, at step 5310. Similar to the first drying step (i.e., step 5302), in some embodiments step 5310 may involve placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter. Step 5310 is considered a “heat curing” step, as heat is used to dry (i.e., cure) the coating according to some embodiments. Next, in some embodiments, the positive or negative pressure source is disconnected and the dry catheter is removed from the oven, at step 5312.

At this point, in some embodiments, the process again diverges into two different options. In some embodiments, one option is to apply a second coat of hydrophilic coating to the inner surface of the catheter, at step 5314. In some embodiments, the other option is to apply a topcoat to the inner surface of the catheter, at step 5316. Similar to the application of the first coat of hydrophilic coating (at step 5306) and the application of the basecoat (at step 5308), in some embodiments, both steps 5314 and 5316 may use positive or negative pressure to fill the catheter with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time.

Next, in some embodiments, the process continues with placing the catheter back into the oven to dry (or “heat cure”) again, at step 5318. Like the first and second drying steps (step 5302 and step 5310), in some embodiments, step 5318 may involve placing the catheter in an oven set to a temperature between 0° C. and 400° C. and/or applying positive or negative pressured air (e.g., oxygen, a mix of oxygen and nitrogen, etc.) to the hub of the catheter in order to dry the inner surface of the catheter. In some embodiments, the process concludes by disconnecting the positive or negative pressure source and removing the dry, coated catheter from the oven, at step 5320.

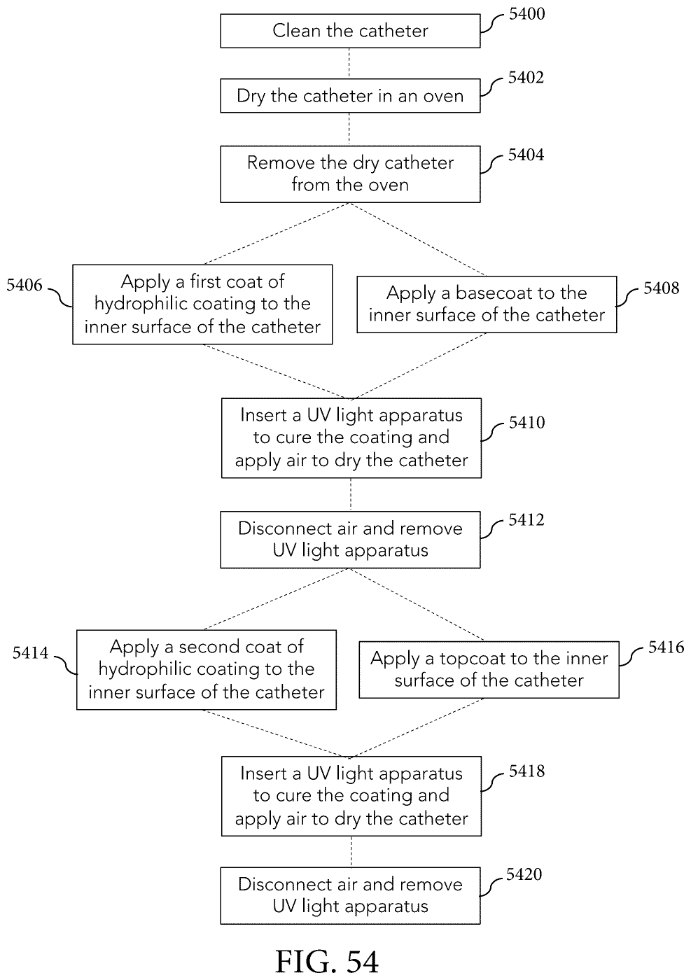

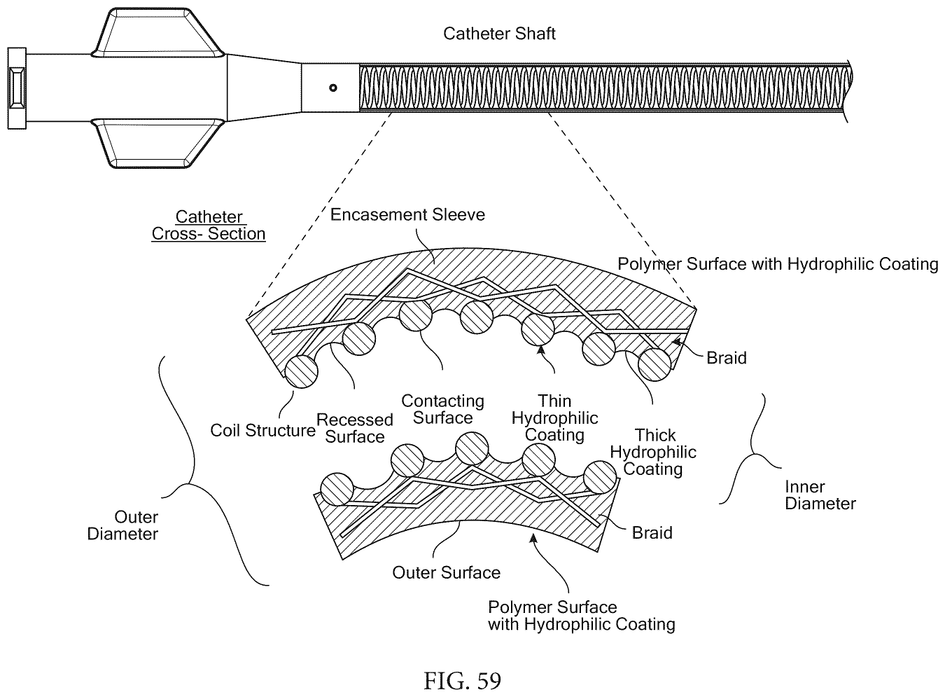

FIG. 54 is similar to FIG. 53 and includes a flowchart illustrating a slightly different non-limiting example process of coating and curing an inner surface of a catheter according to some embodiments. As with the process shown in FIG. 53, for the purposes of this disclosure, the “catheter” recited in FIG. 54 may comprise elements of the catheter system described in FIG. 37-44 (i.e., the outer catheter or the inner catheter), elements of the catheter system described in FIG. 45-49 (i.e., the outer catheter or the inner catheter), elements of the catheter system described in FIG. 50-52, and/or catheter shafts described in FIG. 59. The steps of the process should be considered as applying to any of the catheters recited in this disclosure according to some embodiments.

In some embodiments, the process shown in FIG. 54 starts with cleaning the catheter, at step 5400. In some embodiments, cleaning the catheter includes flushing the catheter with purified water, IPA, a mix of IPA and water, or some other suitable cleansing fluid. In some embodiments, the next step is to dry the catheter in an oven, at step 5402. The drying step may include placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter according to some embodiments. In some embodiments, the process continues with step 5404: removing the dry catheter from the oven.

Next, in some embodiments, the process can continue in one of two possible steps. In some embodiments, one option is to apply a first coat of hydrophilic coating to the inner surface of the catheter, shown at step 5406. In some embodiments, a basecoat is applied to the inner surface of the catheter, at step 5408. In some embodiments, both steps 5406 and 5408 may use positive or negative pressure to fill the catheter with either the hydrophilic coating (step 5406) or the basecoat (step 5408). In some embodiments, the catheter is filled with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time. In some embodiments, a reduction in flow is used to increase the dwell time within a catheter.

After either step 5406 or step 5408, in some embodiments, the process may continue by inserting a UV light apparatus to cure the coating and applying positive or negative pressured air (e.g., oxygen, a mix of oxygen and nitrogen, etc.) to the hub of the catheter in order to dry the inner surface of the catheter, at step 5410. In some embodiments, the UV light apparatus is inserted into the inner diameter of the catheter to cure the coating on the inner surface. Next, the positive or negative pressure source is disconnected and the UV light apparatus is removed from the catheter, at step 5412, according to some embodiments.

At this point, in some embodiments, the process again diverges into two different options. In some embodiments, one option is to apply a second coat of hydrophilic coating to the inner surface of the catheter, at step 5414. In some embodiments, the other option is to apply a topcoat to the inner surface of the catheter, at step 5416. Similar to the application of the first coat of hydrophilic coating (at step 5406) and the application of the basecoat (at step 5408), in some embodiments, both steps 5414 and 5416 may use positive or negative pressure to fill the catheter with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time.

Next, in some embodiments, the process continues with another round of UV light curing, at step 5418. Like the first UV curing step (step 5410), step 5418 may involve inserting a UV light apparatus to cure the coating and applying positive or negative pressured air (e.g., oxygen, a mix of oxygen and nitrogen, etc.) to the hub of the catheter in order to dry the inner surface of the catheter. In some embodiments, the UV light apparatus is inserted into the inner diameter of the catheter to cure the coating on the inner surface. In some embodiments, the process includes disconnecting the positive or negative pressure source and removing the UV light apparatus from the catheter, at step 5420.

The catheter system illustrated in FIG. 37-54 is configured for use in various procedures conducted in a variety of locations of a patient's anatomy. Though brain-specific thrombectomy is discussed, the disclosure should not be considered limiting to any specific type or location of the procedure. The catheter system is used for the aspiration of clots throughout a patient's body, and the various aspects of the catheter system discussed above may improve the rate of clot removal in a number of procedure locations.

Catheter systems may include a full outer catheter and partial inner catheter, like the catheter system shown in FIG. 45-49, or may include a full outer catheter and a full inner catheter, like the catheter system shown in FIG. 50-52. In some embodiments, a catheter system includes a partial outer catheter and a full inner catheter. In some embodiments, a catheter system may also include a partial outer catheter and a partial inner catheter.

A method of using the catheter system described herein may include inserting an outer catheter into a patient's vasculature, wherein the outer catheter includes a proximal end and a distal end located opposite the proximal end, advancing the outer catheter through the patient's vasculature toward a vascular lesion, and advancing the outer catheter to a location selected from the group consisting of a first location and a second location. In some embodiments, the first location is within a first predetermined distance from the vascular lesion, and the second location is within a second predetermined distance from the vascular lesion. In some embodiments, when the outer catheter is in the first location, the outer catheter is able to aspirate the vascular lesion, and when the outer catheter is in the second location, the outer catheter is unable to aspirate the vascular lesion. In some embodiments, when the outer catheter is in the first location, the method further comprises aspirating the vascular lesion with the outer catheter. In some embodiments, when the outer catheter is in the second location, the method may further comprise advancing an inner catheter through the outer catheter toward the first location. In some embodiments, when the inner catheter is in the first location, the method further comprises aspirating the vascular lesion with the inner catheter. In some embodiments, the inner catheter may be advanced over a guidewire having a multi-edge profile configured to reduce friction and improve torque transmission during navigation through tortuous anatomy. In some embodiments, the inner catheter is configured to be a diagnostic catheter. The guidewire may include a helical twist with a pitch ranging from approximately 0.0100 to 0.7500 inches, radiused edges between 0.0010 and 0.0200 inches, and a polymer jacket with a hydrophilic coating to enhance lubricity.

In some embodiments, a fluoropolymer-free manufacturing process for the catheter begins by selecting a PTFE mandrel that matches the desired inner diameter of the catheter. In some embodiments, one or more reinforcement structures are placed on and/or over the mandrel. In some embodiments, a reinforcement structure includes a wire, string, coil, and/or laser-cut hypotube. In some embodiments, different reinforcement structures are placed in different regions of the mandrel and/or are overlaid on each other. In some embodiments, the reinforcement structure includes one or more of a flat surface, a round surface, or some combination of the two.

In some embodiments, the reinforcement structure includes a coil structure, which may include elements such as one or more flat wires and/or one or more round wires as discussed above. While some embodiments may describe the use of one element type (e.g., wire, string) or element shape (e.g., round, flat), it is understood that the specific elements and/or element shapes are interchangeable when describing the metes and bounds of the system, and reference to an “element” is a reference to any combination of element types and/or shapes described herein.

In some embodiments, to form the coil structure, a wire is wound over the polytetrafluoroethylene (PTFE) mandrel using a chosen element, shape, and/or pitch, after which the coil structure is terminated. In some embodiments, the coil structure is configured to leave recesses in the inner diameter. In some embodiments, the coil structure is configured to impart a rib pattern comprising peaks and valleys on the inner diameter of the catheter.

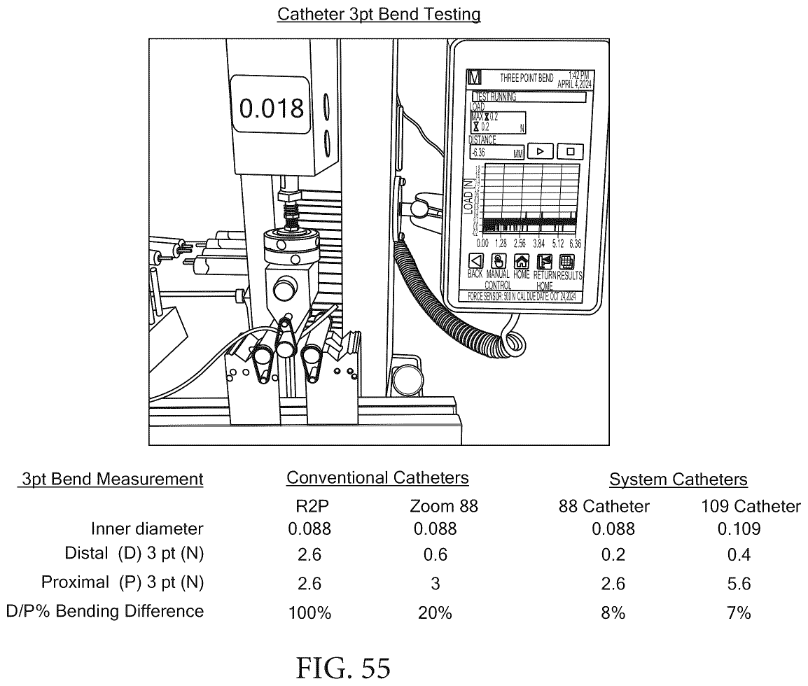

In some embodiments, a braid is then applied over the coiled PTFE mandrel, utilizing the selected material, braid pattern, and braid picks per inch (PPI) to obtain a desired structural integrity. However, it has been found that the coil structure alone, in accordance with some embodiments, provides sufficient properties to achieve the results shown in FIG. 55-58.

In some embodiments, the reinforcement structure is wound around the mandrel in a braided pattern. In some embodiments, the braided pattern is configured to leave recesses in the inner diameter. A non-limiting recess shape includes a polygon shape, which may include a diamond or square pattern and/or grid pattern according to some embodiments. Braiding patterns formed using Steeger USA® machines have been found to produce acceptable results and provide a variety of patterns including flat braids, square braids, spiral braids, strands, and coils.

In some embodiments, the recess shape for any reinforcement structure described herein is configured to create peaks and valleys along the inner diameter of the catheter, where the valleys are configured to reduce the contact area of the inner diameter by 15-85%. In some embodiments, the peaks define the inner diameter contact area for a substantially smooth portion of a traversing structure, such as a guidewire. In some embodiments, the peaks include a flat surface, such as in the case of a flat wire coil structure and/or hypotubes. Flat wires may increase stiffness and/or compressive strength (including vacuum strength) but may have greater contact area against a surface of a traversing structure according to some embodiments. Round wires may decrease stiffness and/or compressive strength but generate less friction for a traversing structure due to a smaller surface area at a round peak as compared to a flat peak. In some embodiments, when a guidewire with a multi-edge profile is used, the valleys formed by the reinforcement structure further reduce frictional engagement, complementing the helical twist of the guidewire to improve torque transmission and trackability.

In some embodiments, the reinforcement structure includes one or more hypotubes. In some embodiments, a hypotube may include one or more hollow portions. In some embodiments, the hollow portions include material removed from the hypotube in a pattern shape. In some embodiments, a step includes creating a pattern shape in the form of one or more holes, longitudinal slots, spiral (helical) cuts, circular (ring) cuts, intersecting grids (e.g., mesh, crisscross lines), and/or any custom geometric pattern. In some embodiments, pattern shapes are configured to impart specific functionality such as expansion, flexibility, or kink resistance.

In some embodiments, the use of laser cutting enables the creation of precise and intricate patterns along the hypotube. Laser cutting provides clean cuts with minimal burrs and heat-affected zones, ensuring the structural integrity and smoothness of the tube in accordance with some embodiments. In some embodiments, the hypotubes are made from biocompatible metals, such as stainless steel or nickel-titanium alloys (Nitinol), which offer excellent strength and flexibility, as well as resistance to corrosion.

In some embodiments, during the reinforcement structure forming step, a platinum iridium marker band is positioned over the braid. In some embodiments, the ends of the braid are trimmed flush with the marker band, and the braid is bonded to the marker band using a urethane-based adhesive. This ensures the marker band is securely attached and the transition between materials is smooth in some embodiments.

In some embodiments, a next phase includes loading each of the polymer extrusions, or tubes, over the coiled and braided reinforcement structure while on the mandrel. In some embodiments, this process starts with the stiffest polymer (e.g., ML24) and concludes with the softest (e.g., 42A Neusoft), which is placed adjacent to the marker band. This step, in some embodiments, creates a gradient of flexibility along the length of the catheter, providing both reinforcement and pliability where needed.

In some embodiments, an expanded fluorinated ethylene propylene (FEP) heat shrink is then loaded over the polymer extrusions, and the assembly is placed into a reflow machine. In some embodiments, the reflow machine employs heated forced air to melt the polymer extrusions, causing them to fuse together and bond to the metallic reinforcement structure. During this process, at least a portion of the polymer flows through the hollow portions of the reinforcement structure. In some embodiments, at least a portion of the polymer coats an inner diameter of the reinforcement structure, sealing the catheter. In some embodiments, at least a portion of the inner diameter is not coated by the polymer, and/or at least a peak surface of the inner diameter is not coated with a polymer. In some embodiments, a peak surface of the inner diameter is coated with a polymer. In some embodiments, polymer located in the area of a hollow portion creates a valley on the inner diameter of the catheter.

After the reflow process, the FEP heat shrink is removed, and the PTFE mandrel is extracted from the assembly according to some embodiments. In some embodiments, the formed catheter does not include PTFE and/or a PTFE liner along the inner diameter. Some embodiments include a step to overmold a hub onto the stiff (e.g., ML24) end of the tube, creating a secure connection point for the catheter. In some embodiments, the hub is attached to the catheter before any hydrophilic coating is applied to the catheter as described above. In some embodiments, an extended portion of polymer material is left in front of the marker band to facilitate subsequent hydrophilic coating processing, which will enhance the catheter's lubricity and ease of use during medical procedures.

In some embodiments, the catheter is then prepared for the hydrophilic coating processing by cleaning the outer diameter, which may include using a wipe saturated with a cleaning agent (e.g., 70% isopropyl alcohol (IPA)) to ensure a clean surface for coating adherence. Some embodiments include a step of coupling a fluid source (e.g., 20cc syringe) to the catheter at the hub and flushing the catheter interior with a cleaning agent (e.g., 70% IPA) to remove any contaminants that may interfere with the coating process.

Following the flush, a hydrophilic basecoat is aspirated into the catheter using a vacuum pump (e.g., another 20cc syringe) to coat the inner diameter of the catheter. To coat the outer diameter, the catheter is at least partially submerged into a container filled with hydrophilic fluid to a specific depth, which is carefully controlled to achieve the desired coating thickness according to some embodiments. Once the desired coating depth is reached, the vacuum is released, enabling the hydrophilic fluid to drain from the inner diameter in some embodiments. In some embodiments, the catheter is withdrawn from the hydrophilic fluid at a consistent rate to ensure an even coating. In some embodiments, the rate of withdrawal affects surface tension which determines coating thickness.

The catheter is then placed in an oven set to a curing temperature of 50-70° C. (e.g., 60° C.) for a duration of 20-40 minutes (e.g., 30 minutes) according to some embodiments. In some embodiments, an airline is connected to the hub during this time, which facilitates the curing of the hydrophilic basecoat on both the inner and outer diameters simultaneously. In some embodiments, air is slowly forced through the catheter. By adjusting the rate of air flow, in some embodiments, it is possible to control the thickness of the hydrophilic coating on the inner diameter. A slower air flow allows more time for the coating to set, potentially leading to a thicker coating, while a faster air flow thins out the coating and/or speeds up the drying process according to some embodiments.

In some embodiments, the process is repeated for a hydrophilic topcoat. In some embodiments, a 20cc syringe is used to aspirate the topcoat into the catheter, which is then dipped into a container of hydrophilic topcoat to the predetermined depth to achieve the desired coating thickness. After reaching the desired depth, in some embodiments the catheter is flushed (e.g., by releasing the vacuum and/or syringe) to remove any excess topcoat and then removed steadily.

In some embodiments, the catheter is placed back into the oven at a curing temperature of 50-70° C. (e.g., 60° C.) for a duration of 20-40 minutes (e.g., 30 minutes). In some embodiments, the airline is still connected to the hub and/or the airline is connected to the hub. In some embodiments, air is supplied to the inner diameter at a same or similar temperature as the curing temperature, allowing the hydrophilic topcoat to cure on both the inner and outer diameters of the catheter simultaneously. In some embodiments, the sacrificial extended tip of the catheter is cut off, and the tip is rounded to ensure a smooth and/or finished end ready for medical use.

In some embodiments, the fluoropolymer-free manufacturing process includes constructing a catheter system that is compatible with hydrophilic coatings. In some embodiments, the catheter structure and/or manufacturing process does not include the use of fluoropolymers within the catheter and instead utilizes a range of materials that are conducive to the application of hydrophilic coatings.

In some embodiments, the catheter's reinforcement structure may include a stainless steel coil, with alternative material options such as nitinol or tungsten. As discussed previously, the reinforcement structure (e.g., coil structure) cross-section can be either round or flat in shape according to some embodiments. Similarly, the catheter may include a stainless steel braid for additional reinforcement in some embodiments, with the same alternative material options and the choice between round and/or flat types of braid. In some embodiments, as an alternative and/or in addition to using coil and/or braid components, the construction may employ a laser-cut hypotube made from stainless steel or nitinol. Depending on application requirements, the catheter may feature just a coil, just a braid, or just a hypotube for reinforcement.

In some embodiments, the termination of the reinforcement structure is achieved using a urethane-based UV adhesive, ensuring a secure end. In some embodiments, the catheter's tubing transitions from rigid to soft materials, starting with Nylon tubing, specifically Vestimid ML24, which is the most rigid polymer used in catheters, followed by Vestimid ML21, the second most rigid polymer, according to some embodiments.

In some embodiments, to create a smooth transition in flexibility, PEBAX tubing is used in in a generally central portion of the device. The PEBAX material comes in varying hardness levels, denoted by the “D” hardness scale, with higher numbers indicating greater rigidity. In some embodiments, the catheter utilizes PEBAX tubing in descending order of rigidity, from 72D to 25D.

For the tip of the device, which requires maximum navigability, urethane tubing is employed in some embodiments. The “D” hardness scale intersects with the “A” hardness scale at 25D, approximately equivalent to 80A. In some embodiments, the urethane tubing used progresses from 25D at the proximal end (i.e., adjacent to the hub) to the softer 42A Neusoft as the distal end. In some embodiments, at the very tip (distal end) of the catheter, a platinum-iridium marker band is incorporated. This marker band allows physicians to visually confirm the catheter's tip location under fluoroscopy according to some embodiments.

In some embodiments, the catheter's female hub connector is constructed by overmolding 72D PEBAX onto the nylon end of the device. Alternatively, a pre-molded hub is attached using UV adhesive in some embodiments. To ensure a smooth transition from the nylon shaft to the 72D PEBAX hub, polyolefin heat shrink is used as a strain relief in some embodiments, providing a seamless and secure connection. In some embodiments, the hydrophilic coating gets pushed through using the hub. In some embodiments, an inner diameter of the hub is also coated with a hydrophilic coating as a result of this process, further reducing friction. Conventional catheters do not include hydrophilic-coated hubs, as conventional manufacturing processes require catheters to be coated before any hydrophilic coating is applied.

In some embodiments, the catheter includes a substantially smooth outer diameter (OD) surface. In some embodiments, the OD surface has a texture depth less than 0.002 inches.

In some embodiments, one or more catheters described herein include a spiral pattern of offset surfaces configured to enhance the lubricity of the inner diameter. In some embodiments, the contacting surface of the catheter, which interfaces with traversing devices (e.g., guidewires, catheters) during delivery or with blood clots during aspiration, includes a width between 0.001 inches and 0.004 inches from one contact surface to another. In some embodiments, the contacting surface comprises materials selected from a group comprising stainless steel, nitinol, nylon, Pebax, and Pellethane as previously described.

In some embodiments, the catheter inner diameter (ID) includes a recessed surface (i.e., valley) formed between the polymer and reinforcement structure, set back from the contacting surface by a distance (i.e., depth) ranging from 0.0001 inches to 0.010 inches. In some embodiments, the recessed surface has a width between peaks of 0.001 inches and 0.007 inches, where the recessed surface defines the pitch of the coil structure. In some embodiments, the recessed surface includes the polymer material and/or the hydrophilic coatings previously described. In some embodiments, both the contacting and recessed surfaces are coated with a hydrophilic layer(s) that may include one or more of polyvinylpyrrolidone (PVP), polyacrylic acid (PAA), or hyaluronic acid (HA), as non-limiting examples.

In some embodiments, the resulting catheter includes a hydrophilic coating with varying thickness along its length. In some embodiments, one or more contacting surfaces include a thin hydrophilic coating with a thickness from approximately 0.0001 inches to 0.000198 inches. In some embodiments, non-contacting areas (recessed areas) include a thick hydrophilic coating ranging from 0.0002 inches to 0.003 inches. In some embodiments, surface tension keeps the hydrophilic coating in place while curing, where more hydrophilic coating will gather in the recessed area, resulting in an inner diameter with alternating hydrophilic coating thickness.

In some embodiments, the catheter is configured to withstand vacuum forces up to 29.92 inches Hg and maintain a circular shape or not collapse more than 30% of its original dimension. In some embodiments, the catheter is configured to withstand a minimum burst pressure of 300 KPA (42.5 psi) (stable up to 100 psi) for a duration of 30 seconds while not leaking and/or substantially maintaining shape. In some embodiments, the catheter's construction is configured to provide tip softness and kink resistance as further described herein.

As previously mentioned, in some embodiments, the catheter includes hydrophilic-coated hubs configured to facilitate the delivery of interventional devices and/or to minimize friction during clot removal. In some embodiments, a method of identifying hydrophilic-coated hubs includes coloring the hub with Tantalum Blue dye to enhance visibility and identification of the hydrophilic coating process.

In some embodiments, the catheter includes a hydrophilic-coated inner diameter hub, which may include polycarbonate or PVACS hubs with a hydrophilic coat. In some embodiments, the coating on the interior of the hub is configured to increase lubricity, further facilitating the delivery of devices through the catheter. In some embodiments, the hub design supports smooth passage of guidewires, including multi-edge profile guidewires with helical twists, which reduce friction and improve torque transmission during navigation.

In some embodiments, the catheter's hypotubes are manufactured using laser cutting techniques to create a spiral pattern. In some embodiments, the laser cuts are of a specific size and distance apart, which promotes inner lubricity. In some embodiments, the precise laser cutting technique, combined with the hydrophilic coating, provides the necessary lubricity for the catheter's inner surfaces. In some embodiments, the offset, repeating patterns in the inner diameter of the catheter enhance lubricity and allow for the elimination of a PTFE liner.

FIG. 55 shows results for a 3-point bending test performed on conventional catheters and system catheters according to some embodiments. In some embodiments, the 3-point test can be used to determine flexural strength, flexural modulus (stiffness), and/or elasticity. In some embodiments, results show stiffness characteristics for a proximal section (closer to the hub) and a distal section (closer to the end) of conventional and system catheters. In some embodiments, using the system and methods described herein, a ratio between the stiffness of the distal section and the proximal section is 15% or less, which is much less than conventional catheters. In some embodiments, a distal/proximal ratio for one or more catheters described herein is between approximately 5-10%.

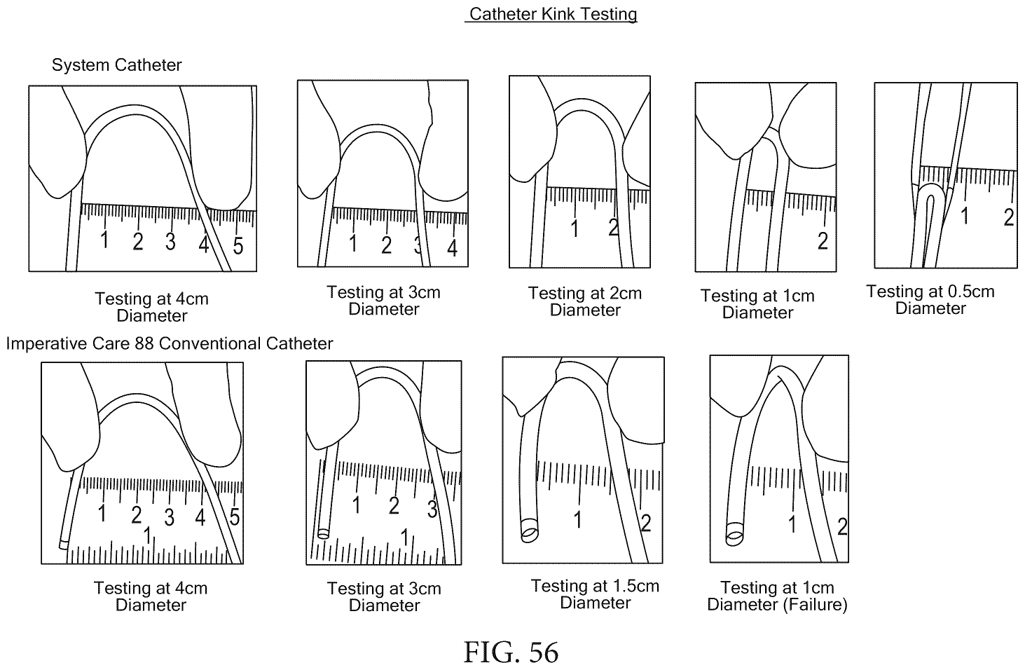

FIG. 56 illustrates a catheter kink test for a system versus a conventional catheter according to some embodiments. Kinking, in the context of this disclosure, refers to the undesirable bending, twisting, and/or creasing of a tube structure (i.e., catheter) to such an extent that it obstructs and/or blocks the motion of a fluid and/or a traversing structure moving within the tube structure. Any reference to “kinking” in this disclosure can be replaced with “collapsing” and/or “creasing” when defining the metes and bounds of the system. In addition, kinking includes a decrease in inner diameter of more than 30% along a first axis of a catheter cross-section, and/or an increase in inner diameter of more than 30% along a second axis of a catheter cross-section.

As shown in FIG. 56, conventional catheters kink and/or collapse at diameter ranges of less than 1.5 cm, where the diameter is measured from one outside portion of the catheter to the other. In contrast, in some embodiments, system catheters are configured to bend to diameter ranges less than 1.5 cm without kinking. In some embodiments, system catheters are configured to bend to diameter ranges less than 1.0 cm without kinking. In some embodiments, system catheters are configured to bend to diameter ranges less than 0.5 cm without kinking, which is far superior to any conventional catheter. In some embodiments, system catheters are configured to bend to a diameter range between 1.5 cm and 5 cm.

In some embodiments, system catheters are configured to bend to an inner radius of curvature less than 16 mm without kinking. In some embodiments, system catheters are configured to bend to an inner radius of curvature less than 10 mm without kinking. In some embodiments, system catheters are configured to bend to an inner radius of curvature less than 5 mm without kinking. In some embodiments, system catheters are configured to bend to an inner radius of curvature less than 2 mm without kinking. In some embodiments, system catheters are configured to bend to an inner radius of curvature less than or equal to 1 mm without kinking, which corresponds to the 0.5 cm test shown in FIG. 56. In some embodiments, system catheters are configured to bend to an inner radius of curvature range of 15 mm to 1 mm.

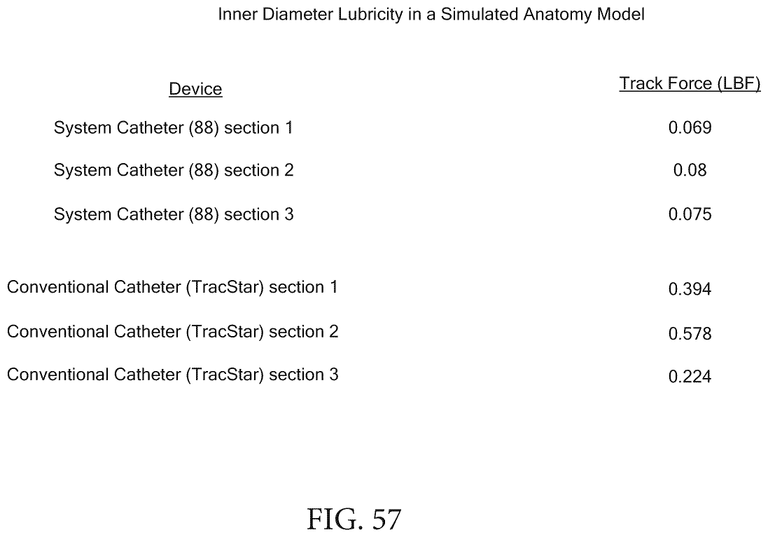

FIG. 57 shows data for inner diameter lubricity in a simulated anatomy model according to some embodiments. Inner diameter lubricity testing in a simulated anatomy model is a method used to evaluate the ease of movement (or lubricity) of devices, such as guidewires or other instruments within the lumen of a catheter, where devices need to be inserted and maneuvered through the catheter without causing damage or discomfort. In some embodiments, this test simulates the conditions that the catheter would experience in the body, including the presence of bodily fluids and the various twists and turns of the vascular system. The goal is to ensure that the catheter's inner surface is sufficiently lubricious to allow smooth, unimpeded movement of devices within it in accordance with some embodiments.

In some embodiments, the catheter is prepared by ensuring it is clean and free of any debris. In some embodiments, a lubricant, often a saline solution or a specific medical-grade lubricant, is applied to the device that will be inserted into the catheter, simulating the presence of bodily fluids. In some embodiments, a traversing structure (e.g., a guidewire) is inserted into the catheter and maneuvered through it, where the ease of movement is observed and recorded. This can be done manually or using a machine that can apply a consistent force according to some embodiments. In some embodiments, the force required to move the device through the catheter is measured using a force gauge or similar instrument. The lower the force required to move the traversing structure, the more lubricious the catheter's inner diameter is considered to be in accordance with some embodiments.

As shown in FIG. 57, in some embodiments, track forces for system catheters are significantly less than conventional catheters in similar sections. In some embodiments, a track force for system catheters is less than 0.2 lbs force (lbf) for any catheter section. In some embodiments, a track force for system catheters is less than 0.1 lbf force for any catheter section. In some embodiments, a track force for system catheters falls within a range of 0.2 lbf to 0.05 lbf force for any catheter section. These results demonstrate improved lubricity, which enhances compatibility with advanced guidewires, including multi-edge profile guidewires featuring helical twists and hydrophilic coatings for reduced friction and improved torque transmission.

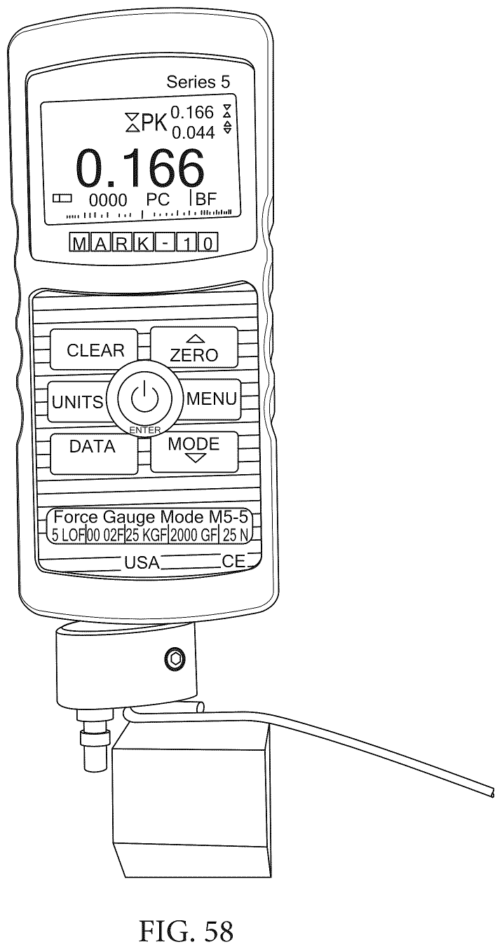

FIG. 58 shows a measured catheter distal looped compression force according to some embodiments. In some embodiments, the catheter is configured to provide a Catheter Distal Looped Compression Force of 0.009-0.300 lbf. In some embodiments, catheter tip spring-back force is measured by bending the distal 2 cm to 90 degrees and measuring with a force gauge the spring-back force as it returns to straight. In some embodiments, spring-back force of the distal end (tip) includes a range of 0.001-0.100 lbf.

FIG. 59 shows a cross-sectional view of a catheter shaft according to some embodiments. In some embodiments, the catheter shaft, which may be a part of any catheter described herein and/or shown in the FIGs., includes a coil structure formed from one or more elements such as wire, string, or laser-cut hypotube as previously described in accordance with some non-limiting embodiments. In some embodiments, the coil structure, in combination with an encasement sleeve, is configured to form recesses along the inner diameter of the catheter shaft. These recessed surfaces are set back from the contacting surface in the ranges previously described according to some embodiments.

The contacting surface, which contacts traversing device surfaces during delivery and/or blood clots during aspiration, includes a thin hydrophilic coating with a thickness from approximately 0.0001 inches to 0.000198 inches in some embodiments. In some embodiments, the recessed surface includes a thick hydrophilic coating ranging from 0.0002 inches to 0.003 inches.

In some embodiments, the catheter shaft includes a braid. In some embodiments, the braid is applied over the coiled structure. In some embodiments, only the braid is used, where areas between the braid define the recessed surfaces. The braid, which is formed from a selected material, braid pattern, and braid picks per inch (PPI), contributes to the desired structural integrity of the catheter shaft.

In some embodiments, the catheter shaft includes an encasement sleeve which may include one or more polymers described herein. Once the encasement sleeve is coupled and/or fused to the reinforcement structure, which includes the coil structure and/or braid in this non-limiting example, the combination of the reinforcement structure and the encasement sleeve creates peaks and valleys within the inner diameter of the formed catheter shaft. In some embodiments, as the encasement sleeve is deformed during the coupling process, at least part of the encasement sleeve flows through the hollow portions formed by the reinforcement structure. In some embodiments, once coupled, the catheter shaft includes a smooth outer surface defining the limits of the outer diameter which is smoother than the surface of the inner diameter, wherein each surface is coated with at least one hydrophilic coating, enhancing the catheter's lubricity and ease of use during medical procedures. In some embodiments, the inner diameter is configured to accommodate advanced guidewires, including the multi-edge profile guidewires with helical twists described herein, which reduces friction and improves torque transmission during navigation through tortuous anatomy.

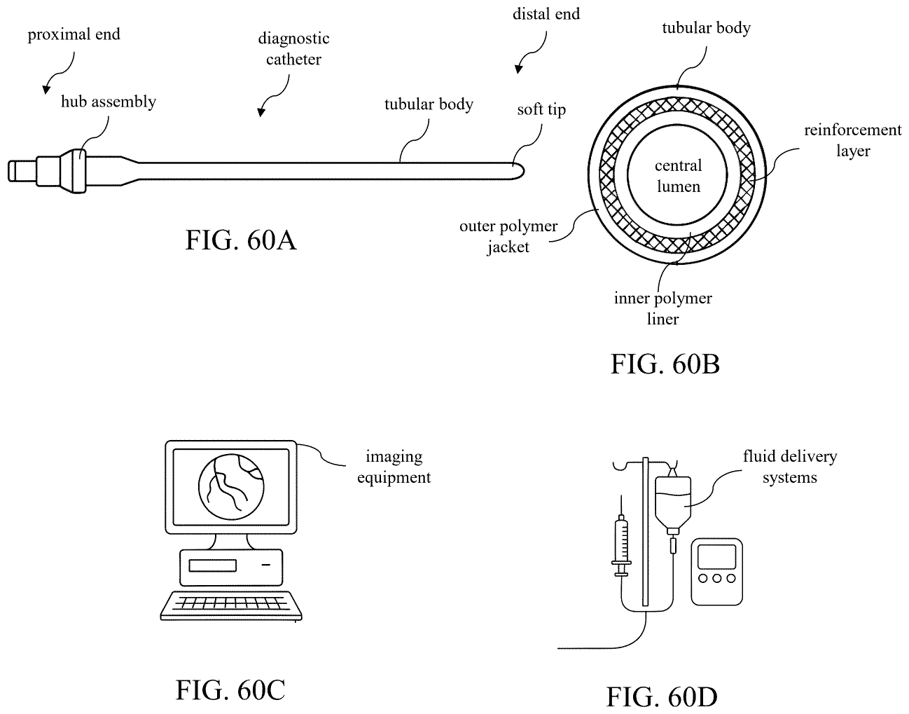

As shown in FIGS. 60A and 60B, in some embodiments, the system includes a diagnostic catheter configured for intravascular navigation and imaging. In some embodiments, the diagnostic catheter includes an elongated tubular body extending from a proximal end to a distal end, the tubular body defining a central lumen sized to accommodate guidewires and other interventional devices. The proximal end may include a hub assembly with a connector for interfacing with imaging equipment (FIG. 60C) and fluid delivery systems (FIG. 60D), which also forms a portion of the system. Structurally, the diagnostic catheter may incorporate a multi-layer wall construction comprising an inner polymer liner, a reinforcement layer, such as braided or coiled metallic filaments, and an outer polymer jacket, providing torque transmission, pushability, and kink resistance while maintaining flexibility for navigation through tortuous anatomy. The distal end may include a soft tip segment to reduce trauma during advancement. In some embodiments, the soft tip segment of the diagnostic catheter may include a length ranging from approximately 3 centimeters to 15 centimeters measured from the distal end, where the specific length may vary based on catheter size and intended application. In some embodiments, the soft tip comprises a polymer material having a durometer between approximately 25D and 42A on the Shore hardness scale, wherein lower values correspond to increased softness and enhanced flexibility for atraumatic navigation through tortuous anatomy.

In some embodiments, the diagnostic catheter is configured to facilitate imaging and diagnostic assessment by enabling contrast injection through the central lumen, provide a stable conduit for guidewire placement and device delivery, transmit torque and axial force for precise positioning within vascular pathways, and maintain structural integrity under physiological pressures and during manipulation. In some embodiments, the diagnostic catheter may be used in conjunction with the guidewire described herein and/or the catheter described above in relation to FIG. 59. When implemented as a method of use, the system streamlines femoral access procedures (or any procedure that uses a diagnostic catheter, reducing the number of exchanges compared to conventional workflows.

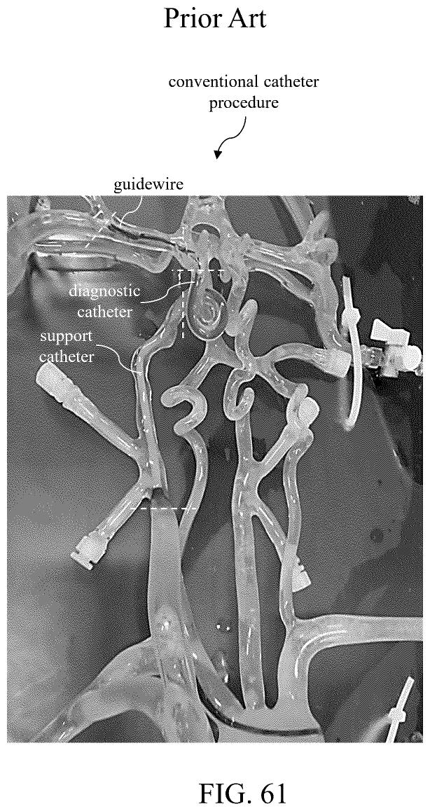

For example, as illustrated in prior art FIG. 61, conventional femoral access procedures involve multiple device exchanges and incremental steps to achieve adequate support and navigation, in accordance with some embodiment. The procedure often begins with introducing a large-bore guiding catheter (e.g., Zoom 88) to establish initial access. A diagnostic catheter (e.g., 6F Diagnostic Catheter) is then advanced for imaging and anatomical assessment.

Following diagnostic imaging, a flexible conventional guidewire (non-helical guidewire; e.g., 0.035 Glidewire) is introduced to navigate tortuous anatomy. To increase stability, additional support catheters (e.g., Zoom 71) and progressively stiffer guidewires (e.g., 0.038 Socrates, 0.035 Colossus Wire) are exchanged. Each transition requires careful manipulation, adding complexity, procedural time, and potential risk of complications.

This multi-step approach reflects the limitations of conventional systems, where support and navigation functions are distributed across six different components, necessitating repeated exchanges and repositioning.

In contrast the present system can perform a femoral access procedure (or radial access) with only three components: The guidewire described herein, a diagnostic catheter, and the outer (i.e., textured inner surface) catheter described herein. Even more advantageously, due to the features of the guidewire and outer catheter described herein, all three components can be delivered simultaneously due to the unique bending ability of the system, eliminating the need to add different catheters for support and the need for progressively stiffer guidewires.

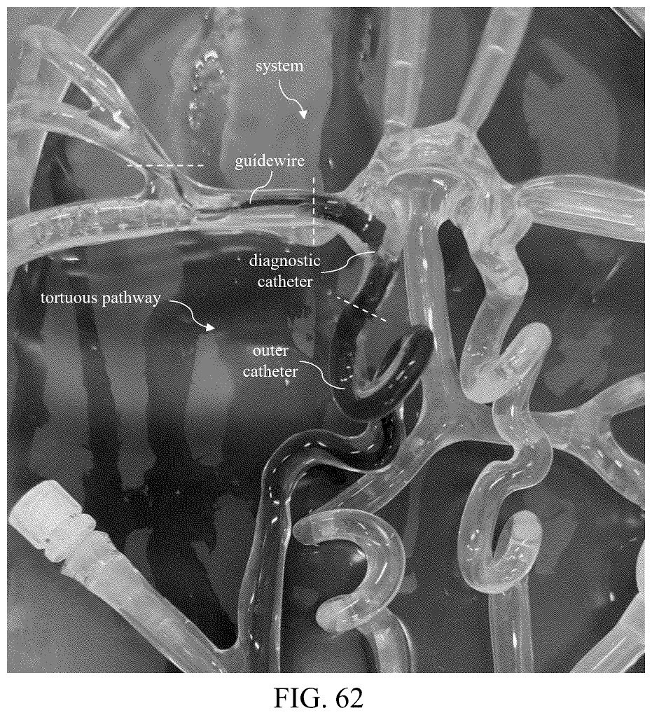

FIG. 62 shows the system advancing through a tortuous pathway in accordance with some embodiments. In some embodiments, a non-limiting example of a tortuous pathway may include a segment of the internal carotid artery extending through the cavernous sinus region, where the vessel exhibits multiple sharp bends and curvature changes within a short axial distance (e.g., 5 cm). In some embodiments, a tortuous pathway may include an S-shaped configuration with alternating angles exceeding 90 degrees, creating a complex trajectory that requires enhanced flexibility and torque control for safe navigation. Such anatomical geometry presents increased frictional engagement and risk of vessel trauma when using conventional devices. Various tortuous pathways may be found throughout different portions of an anatomy.

In some embodiments, the outer catheter is able to go all the way to the to the tortuous pathway without the need for one or more additional support catheters at various stages. In some embodiments, the system may be advanced by advancing the guidewire, a diagnostic catheter, and the outer catheter together to the tortuous pathway. The system may be advanced through the tortuous pathway by advancing the guidewire, advancing the diagnostic catheter, then advancing the outer catheter in an incremental fashion, taking advantage of the reduced friction surfaces the guidewire and/or outer catheter provide.

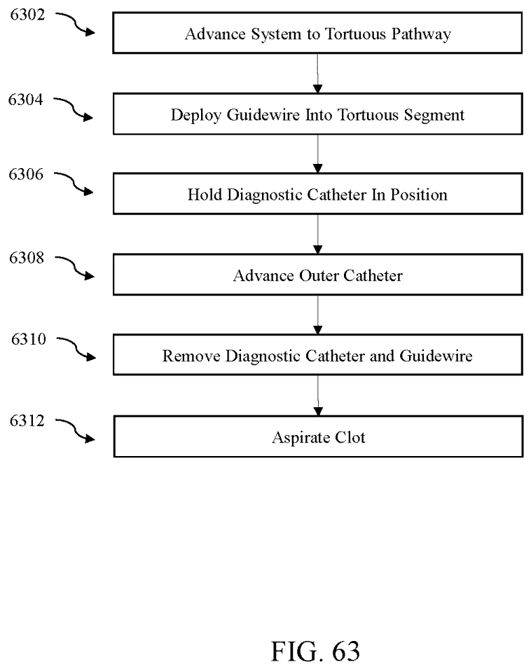

FIG. 63 includes non-limiting method steps for a femoral or radial access procedure, in accordance with some embodiments. In step 6302, a method step includes advancing the guidewire, diagnostic catheter, and outer catheter together through the femoral artery toward the tortuous pathway, in accordance with some embodiments. In some embodiments, the combined advancement minimizes device exchanges and leverages the bending ability of the guidewire and outer catheter to enable initial navigation.

In step 6304, a method step includes deploying the guidewire distally beyond the tortuous segment to establish a stable rail for subsequent catheter positioning and device delivery, in accordance with some embodiments. The guidewire described herein provides superior directional control due to the helical profile.

In step 6306, a method step includes maintaining the diagnostic catheter in position proximal to the tortuous pathway to provide support during advancement, in accordance with some embodiments. In some embodiments, the diagnostic catheter is configured to enable imaging and contrast injection to confirm vessel anatomy and procedural trajectory.

In step 6308, a method step includes advancing the outer catheter over the diagnostic catheter and guidewire until the distal end of the outer catheter reaches the desired anatomical location or the face of an occlusion, in accordance with some embodiments. In some embodiments, the outer catheter benefits from the reduced friction interface provided by the guidewire and diagnostic catheter, allowing controlled navigation through tortuous anatomy without requiring additional support catheters.

In step 6310, a method step includes removing the diagnostic catheter and guidewire after the outer catheter is positioned at the target site, in accordance with some embodiments.

In step 6312, aspirating the clot through the outer catheter by coupling the catheter to a syringe or vacuum source, or alternatively inserting a therapeutic device through the catheter to complete the desired intervention, in accordance with some embodiments.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use the system according to some embodiments. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

For example, the system may be described as a guidewire comprising a proximal end, a distal end, and intermediate section. In some embodiments, the intermediate section is located between the proximal end and the distal end. In some embodiments, the intermediate section includes a multi-edge profile. In some embodiments, each edge of the multi-edge profile extends along the intermediate section from the proximal end to the distal end. In some embodiments, the guidewire is a medical device configured for insertion into a body lumen of a patient.

In some embodiments, the multi-edge profile is twisted into a helical configuration. In some embodiments, the helical configuration comprises a first helical pitch section and a second helical pitch section. In some embodiments, a first pitch of the first helical pitch section is different than a second pitch of the second helical pitch section. In some embodiments, the helical configuration comprises a first helical diameter section and a second helical diameter section. In some embodiments, a first diameter of the first helical diameter section is different than a second diameter of the second helical diameter section.

In some embodiments, at least a portion of the proximal end includes a sectional profile without edges. In some embodiments, at least a portion of the distal end includes a sectional profile without edges. In some embodiments, the proximal end, and the distal end each include a round sectional profile.

In some embodiments, at least a portion of the guidewire includes a polymer jacket. In some embodiments, the polymer jacket comprises a hydrophilic coating configured to reduce friction during intravascular navigation. In some embodiments, at least a portion of the helical configuration does not comprise a polymer jacket.

In some embodiments, the system may be described as comprising a guidewire and a catheter. In some embodiments, the guidewire includes a multi-edge profile. In some embodiments, the catheter includes a textured inner surface. In some embodiments, the guidewire and the catheter are medical devices configured for insertion into a body lumen of a patient. In some embodiments, the catheter includes a textured inner surface that at least partially comprises a shape of a support structure of the catheter.

In some embodiments, the system is used in conjunction with one or more method steps to improve upon prior medical procedures. In some embodiments, a method comprises providing a guidewire that includes a multi-edge profile. Some embodiments include a step of providing a first catheter that includes a central lumen with an inner surface. Some embodiments include a step of inserting the guidewire and catheter together into a tortuous pathway. In some embodiments, the guidewire and the catheter are medical devices configured for insertion into a body lumen of a patient.

Some embodiments include a step of not using more catheters than the first catheter to reach the tortuous pathway. In some embodiments, the first catheter includes a textured inner surface formed from a support structure of the catheter. Some embodiments include a step of providing a diagnostic catheter. Some embodiments include a step of inserting the guidewire, the diagnostic catheter, and the first catheter together into the tortuous pathway.

Some embodiments of the system are presented with specific values and/or setpoints. These values and setpoints are not intended to be limiting and are merely examples of a higher configuration versus a lower configuration and are intended as an aid for those of ordinary skill to make and use the system.

Any text in the drawings is part of the system's disclosure and is understood to be readily incorporable into any description of the metes and bounds of the system. Any functional language in the drawings is a reference to the system being configured to perform the recited function, and structures shown or described in the drawings are to be considered as the system comprising the structures recited therein. It is understood that defining the metes and bounds of the system using a description of images in the drawing does not need a corresponding text description in the written specification to fall with the scope of the disclosure.

Furthermore, acting as Applicant's own lexicographer, Applicant imparts the explicit meaning and/or disavow of claim scope to the following terms:

“Substantially” and “approximately” when used in conjunction with a value encompass a difference of 5% or less of the same unit and/or scale of that being measured (e.g., degrees, volume, mass, distance).

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled”are not restricted to physical or mechanical connections or couplings.

The figures, which are not necessarily to scale, depict some embodiments and are not intended to limit the scope of embodiments of the system.

It will be appreciated by those skilled in the art that while the system has been described above in connection with some embodiments and examples, the system is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the system are set forth in the following claims.

I claim:

1. A guidewire comprising:
a proximal end,
a distal end, and
and intermediate section;
wherein the intermediate section is located between the proximal end and the distal end;
wherein the intermediate section includes a multi-edge profile;
wherein each edge of the multi-edge profile extends along the intermediate section from the proximal end to the distal end; and
wherein the guidewire is a medical device configured for insertion into a body lumen of a patient.

2. The guidewire of claim 1,
wherein the multi-edge profile is twisted into a helical configuration.

3. The guidewire of claim 2,
wherein the helical configuration comprises a first helical pitch section and a second helical pitch section; and
wherein a first pitch of the first helical pitch section is different than a second pitch of the second helical pitch section.

4. The guidewire of claim 2,
wherein the helical configuration comprises a first helical diameter section and a second helical diameter section; and
wherein a first diameter of the first helical diameter section is different than a second diameter of the second helical diameter section.

5. The guidewire of claim 1,
wherein at least a portion of the proximal end includes a sectional profile without edges.

6. The guidewire of claim 1,
wherein at least a portion of the distal end includes a sectional profile without edges.

7. The guidewire of claim 1,
wherein the proximal end and the distal end each include a round sectional profile.

8. The guidewire of claim 2,
wherein at least a portion of the guidewire includes a polymer jacket.

9. The guidewire of claim 8,
wherein the polymer jacket comprises a hydrophilic coating configured to reduce friction during intravascular navigation.

10. The guidewire of claim 8,
wherein at least a portion of the helical configuration does not comprise a polymer jacket.

11. A system comprising:
a guidewire, and
a catheter;
wherein the guidewire includes a multi-edge profile;
wherein the catheter includes a textured inner surface; and
wherein the guidewire and the catheter are medical devices configured for insertion into a body lumen of a patient.

12. The system of claim 11,
wherein the multi-edge profile is twisted into a helical configuration.

13. The system of claim 12,
wherein the helical configuration comprises a first helical pitch section and a second helical pitch section; and
wherein a first pitch of the first helical pitch section is different than a second pitch of the second helical pitch section.

14. The system of claim 12,
wherein the helical configuration comprises a first helical diameter section and a second helical diameter section; and
wherein a first diameter of the first helical diameter section is different than a second diameter of the second helical diameter section.

15. The system of claim 12,
wherein the textured inner surface at least partially comprises a shape of a support structure of the catheter.

16. A method comprising:
providing a guidewire that includes a multi-edge profile;
providing a first catheter that includes a central lumen with an inner surface; and
inserting the guidewire and catheter together into a tortuous pathway;
wherein the guidewire and the catheter are medical devices configured for insertion into a body lumen of a patient.

17. The method of claim 16,
wherein the method further includes not using more catheters than the first catheter to reach the tortuous pathway.

18. The method of claim 16,
wherein the first catheter includes a textured inner surface formed from a support structure of the catheter.

19. The method of claim 16, further comprising:
providing a diagnostic catheter.

20. The method of claim 19, further comprising:
inserting the guidewire, the diagnostic catheter, and the first catheter together into the tortuous pathway.
